(19) United States
(12) Patent Application Publication
(10) Pub. No.: US 2026/0112456 A1
(43) Pub. Date: Apr. 23, 2026

(54) SELECTION OF CHEMICAL SYNTHESIS PROCESSES

(71) Applicant: The Regents of the University of Michigan, Ann Arbor, MI (US)

(72) Inventors: Rui Zhang, Ann Arbor, MI (US); Di Wang, Ann Arbor, MI (US); Timothy A. Cernak, Ann Arbor, MI (US)

(21) Appl. No.: 19/114,242

(22) PCT Filed: Sep. 22, 2023
(86) PCT No.: PCT/US2023/074887

§ 371 Date: Mar. 21, 2025

Related U.S. Application Data
(60) Provisional application No. 63/376,905, filed on Sep. 23, 2022.

(51) Int. Cl. G16C 20/10; G16C 20/70; G16C 20/80

(52) U.S. Cl. CPC G16C 20/10; G16C 20/70; G16C 20/80

(57) ABSTRACT

The present disclosure provides methods, systems, and non-transitory computer-readable media for identifying efficient chemical synthesis processes, using molecular graph editing.

### CROSS-REFERENCE TO RELATED APPLICATIONS

This application claims the benefit of U.S. Provisional Application No. 63/376,905, filed Sep. 23, 2022, the content of which is herein incorporated by reference in its entirety.

### FIELD

The present disclosure provides methods, systems, and non-transitory computer-readable media for identifying efficient chemical synthesis processes, e.g., using molecular graph editing.

### BACKGROUND

Efficient total synthesis converts commercially available starting materials into complex target molecules as quickly as possible. In practice, this is achieved by leveraging high impact key steps, which typically form many of the requisite target bonds simultaneously. Key steps in synthesis are notable as they rapidly generate structural complexity. Common examples include cycloadditions, cascade, or multicomponent coupling reactions.

While the notion of a key step is well known to practitioners of total synthesis, it has not been formalized in computer aided synthesis planning (CASP). Modern CASP strategies aim to minimize protecting group manipulations and maximize convergency, but the focus of automated retrosynthesis has been on encoding reaction rules for maximum reliability in experimental realization of predicted routes, which favors well-precedented reactions that are likely to work. Meanwhile, state-of-the-art synthetic strategies maximize step and atom economy by targeting innovative but riskier key steps, and by minimizing low impact concession steps such as protecting group manipulations, unnecessary redox operations, and functional group interconversions. A system for recognizing synthetic ideality could synergize with the power of modern CASP platforms.

### SUMMARY

This disclosure relates to methods and systems, including computer-implemented methods and non-transitory computer-readable media, for selecting an optimized chemical synthesis process for a given target molecule.

In one aspect, disclosed herein is a method for selecting a chemical synthesis process for a target molecule. In some embodiments, the method is a computer-implemented method.

In some embodiments, the methods comprise: generating adjacency matrices for the target molecule and all synthetic intermediates for one or more routes of synthesis of the target molecule; determining graph edit distances from the adjacency matrices; and identifying a chemical synthesis process. In some embodiments, the methods comprises generating adjacency matrices and determining graph edit distances for two or more routes of synthesis for the target molecule.

In some embodiments, the one or more routes of synthesis comprises a computer aided synthesis planning (CASP) route. In some embodiments, the one or more routes of synthesis comprises an experimentally-determined route of synthesis.

In some embodiments, the two or more routes of synthesis are any combination of CASP and/or experimentally-determined routes of synthesis.

In some embodiments, the methods further comprise determining maximum common substructure (MCS) distance for the target molecule and all synthetic intermediates for each of the one or more routes of synthesis. In some embodiments, the methods further comprise calculating a final distance metric for each of the one or more routes of synthesis based on the maximum common substructure (MCS) distance and the graph edit distances.

In some embodiments, the methods further comprise identifying one or more high impact steps in the one or more routes of synthesis. In some embodiments, the high impact steps maximize the graph edit distance, the MCS distance, and/or the final distance metric between steps in the route of synthesis. In some embodiments, the high impact steps form two or more requisite target bonds simultaneously.

In some embodiments, the methods further comprise neighboring modest to low impact steps in the one or more routes of synthesis. In some embodiments, the methods further comprise grouping the neighboring modest to low impact steps into fewer steps with higher impact. In some embodiments, the neighboring modest to low impact steps are grouped into a single step.

In some embodiments, the identified chemical synthesis process combines high impact steps from two or more different routes of synthesis for the target molecule.

In some embodiments, the identified chemical synthesis process minimizes neighboring modest to low impact steps.

In some embodiments, the identified chemical synthesis process has fewer steps than a previously developed synthesis process for the target molecule.

In some embodiments, identified chemical synthesis decreases reagent costs, utilizes available starting or intermediate compounds or reagents, and/or increases yield when compared to a previously developed synthesis process for the target molecule.

In some embodiments, the methods further comprise synthesizing the target molecule according to the selected chemical synthesis process.

In some embodiments, the methods comprise: generating adjacency matrices for the target molecule and all synthetic intermediates; determining graph edit distances from the adjacency matrices; and selecting a chemical synthesis process based on the graph edit distances.

In some embodiments, the identified chemical synthesis process has fewer steps than a previously developed synthesis process for the target molecule. In some embodiments, the method further comprises identifying one or more high-impact steps in a chemical synthesis process. In some embodiments, the method further comprises synthesizing the target molecule according to the selected chemical synthesis process. In some embodiments, the method is a computer-implemented method.

In one aspect, disclosed herein is a non-transitory computer-readable medium storing instructions, that when executed by one or more processors performs operations of the disclosed methods.

In some embodiments, the non-transitory computer-readable medium storing instructions, that when executed by one or more processors performs operations comprising: generating adjacency matrices for the target molecule and all synthetic intermediates for one or more routes of synthesis of the target molecule; determining graph edit distances from the adjacency matrices; and identifying a chemical synthesis process.

In some embodiments, the operations further comprise one or both of: determining maximum common substructure (MCS) distance; and calculating a final distance metric based on the maximum common substructure (MCS) distance and the graph edit distances. In some embodiments, the operations further comprise any or all of: identifying one or more high impact steps in the one or more routes of synthesis; identifying neighboring modest to low impact steps in the one or more routes of synthesis; and grouping the neighboring modest to low impact steps into fewer steps with higher impact.

In one aspect, disclosed herein is a system comprising: one or more processors; and the computer readable medium described herein.

Other aspects and embodiments of the disclosure will be apparent in light of the following detailed description and accompanying figures.

### BRIEF DESCRIPTION OF THE DRAWINGS

FIG. 1 shows two CASP derived syntheses of stemoamide leveraging a Mannich reaction or Schmidt-Aubé rearrangement as the key step.

FIGS. 2A-2G show information and data regarding identification of key steps by graph edit analysis. FIG. 2A: Network analysis of 50 SYNTHIA™-predicted routes to (−)-stemoamide. In this search result, the Mannich reaction featured as a consistent disconnection, and is highlighted as a cluster of four orange dots in each route. FIG. 2B: An adjacency matrix of compounds 8, 16, 17, 13 and HBr whose width-length is determined by the total number of heavy atoms in the overall synthesis. For concession groups, only the attached atom is considered in the graph so with the PMP-protecting group has 9-heavy atoms, only the first aromatic carbon (attached to nitrogen) is included. Stereocenters are encoded along the diagonal as whether they are present. FIGS. 2C-2D: Adjacency matrices of compounds 19 and 1, respectively. FIG. 2E: Classification of bond type used in graph edit analysis. FIGS. 2F-2G: Graph edit distance plots of a retrosynthetic route to 1 (FIG. 2F), and 3 (FIG. 2G), produced by CASP.

FIG. 3 shows a selected 7-step route to (−)-stemoamide featuring an organocatalyzed asymmetric Mannich reaction as a key step, calculated by SYNTHIA™.

FIGS. 4A-4B show adjacency matrix-encoding of molecules. FIG. 4A: A 3×3 adjacency matrix encoding ethylamine. The pink squares represent bonds between atoms 1 and 2, and atoms 1 and 3. As the matrix is symmetrical about its diagonal, each bond is encoded twice. FIG. 4B: A matrix-encoded synthetic route, forming pyrrolidin-2-one from ethylamine and formic acid. Since the 7-5 bond is broken during the route, it is a concession bond, encoded as a blue square. And since atom 7 does not appear in the final target, it is a concession atom. Color legend: pink is a purchased bond, grey is a strategic bond and blue is a concession bond. See FIG. 2 of main text for additional details.

FIG. 5 shows plots of other molecular similarity metrics computed from 2,048-bit Morgan fingerprints of radius 2.

FIG. 6 shows graph edit distance analyses of six published total syntheses: (+)-welwitindolinone A by Baran (Nature 446, 404-408 (2007)), (+)-frondosin B by Danishefsky (J. Am. Chem. Soc. 123, 1878-1889 (2001)), (+)-englerin A by Christmann (Angew. Chem. Int. Ed. 48, 9105-9108 (2009)), (−)-englerin A by Chain (J. Am. Chem. Soc. 133, 6553-6556 (2011)), (−)-strychnine by MacMillan (Nature 475, 183-188 (2011)) and (±)-strychnine by Vanderwal (Chem. Sci. 2, 649-651 (2011)).

FIGS. 7A-7B show: FIG. 7A: an adjacency matrix analysis of 7-step calculated route to (−)-stemoamide featuring an organocatalyzed asymmetric Mannich reaction as a key step. FIG. 7B: a graph edit distance analysis of the calculated route.

FIG. 8 shows a selected 9-step route to (−)-stemonine featuring a Michael addition—alkylation as a key step, calculated by SYNTHIA™.

FIGS. 9A-9B show: an adjacency matrix analysis of the 9-step calculated route to (−)-stemonine (FIG. 9A); and a graph edit distance analysis of the calculated route (FIG. 9B).

FIGS. 10A-10B show: a total synthesis of (+)-stemoamide in six steps (FIG. 10A); and a graph edit distance analysis of the route (FIG. 10B).

FIGS. 11A-11C show: total synthesis of (−)-stemoamide in three steps (FIG. 11A); graph edit distance analysis of the route (FIG. 11B); and conversion of (−)-stemoamide into (−)-stemonine (FIG. 11C).

FIGS. 12A-12B show: (FIG. 12A): selected 8-step route to (−)-stemoamide, featuring a Michael addition—alkylation as a key step, calculated by SYNTHIA™ with the Mannich reaction excluded; and (FIG. 12B): graph edit distance analysis of the calculated route.

FIGS. 13A-13B show: graph edit distance analysis of the experimental Mannich route to (+)-stemoamide, showing undercutting of steps 3-5 should a hypothetical anti-Markovnikov hydroamidation be possible (FIG. 13A); and the 4-step synthetic route to (+)-stemoamide with a hypothetical anti-Markovnikov hydroamidation (FIG. 13B).

FIGS. 14A-14B show: a 10-step proposed synthetic route to (+)-gelsemine (FIG. 14A); and graph edit distance analysis of the calculated route and (FIG. 14B).

FIGS. 15A-15B show: a 12-step proposed synthetic route to (+)-waihoensene (FIG. 15A); and graph edit distance analysis of the calculated route (FIG. 15B).

FIG. 16 is the SYNTHIA™ predicted route to (−)-stemoamide. SYNTHIA™ recommends when groups should be protected but does not propose exact protecting groups.

FIG. 17 is the SYNTHIA™ predicted route to (−)-stemoamide with a key asymmetric Mannich reaction and atom mapping throughout the route.

FIG. 18 is the SYNTHIA™ predicted route to norstemoamide (ent-27). SYNTHIA™ recommends when groups should be protected but does not propose exact protecting groups.

FIG. 19 is the SYNTHIA™ predicted route to (−)-stemoamide with a key Schmidt reaction of a cyclobutanone intermediate and atom mapping throughout the route.

FIGS. 20A-20B show: FIG. 20A: Adjacency matrix analysis of each starting material set, intermediate and final product in the 7-step calculated route (FIGS. 18 and 19) to (−)-stemoamide, featuring a Schmidt reaction of a cyclobutanone intermediate as a key step. Step 1 was not highlighted as the predicted Diels-Alder reaction was deemed to be unlikely to succeed. FIG. 20B: Graph edit distance analysis of the calculated route.

FIG. 21 is the SYNTHIA™ predicted route to norstemoamide ent-27. SYNTHIA™ recommends when groups should be protected but does not propose exact protecting groups.

FIG. 22 is parameters generating the route for (−)-stemonine developed in FIG. 2G, based on a key Michael addition—allylation.

FIG. 23 is the SYNTHIA™ predicted route to (−)-stemonine with a key Michael addition—alkylation, as in FIG. 8, with atom mapping throughout the route.

FIG. 24 is parameters generating the route for stemoamide (1). A Michael addition—methylation was identified as a key step.

FIG. 25 is the SYNTHIA™ predicted route to (−)-stemoamide with a key Michael addition—alkylation when the Mannich reaction was excluded in the search (as in FIG. 12A), and atom mapping throughout the route.

FIGS. 26A-26B show: FIG. 26A: Adjacency matrix analysis of each starting material set, intermediate and final product in the 8-step calculated route (FIG. 25) to (−)-stemoamide, when the Mannich reaction was excluded in the search. It features an organocatalyzed Aldol—Wittig cascade and a Michael addition—alkylation as key steps, with the latter being incorporated in the experimental Schmidt-Aubé route to (−)-stemoamide (see FIG. 4 for details). FIG. 26B: Graph edit distance analysis of the calculated route.

FIG. 27 is the parameters generating the routes of (+)-gelsemine.

FIG. 28 is a SYNTHIA™ predicted route to (+)-gelsemine, as in FIG. 14A, with an organocatalyzed Mannich reaction, an enyne metathesis, and a [4+2]cycloaddition as key steps, and atom mapping throughout the route.

FIGS. 29A-29B show: FIG. 29A: Adjacency matrix analysis of each starting material set, intermediate and final product in the 10-step calculated route (FIG. 28) to (+)-gelsemine, featuring an organocatalyzed Mannich reaction, an enyne metathesis, and a [4+2]cycloaddition as key steps. FIG. 29B: Graph edit distance analysis of the calculated route.

FIG. 30 is the parameters generating the routes of (+)-waihoensene.

FIG. 31 is SYNTHIA™ predicted route to (+)-waihoensene, as in FIG. 15A, with a Michael addition—allylation and a [4+2]cycloaddition as key steps, and atom mapping throughout the route.

FIGS. 32A-32B show: FIG. 32A: Adjacency matrix analysis of each starting material set, intermediate and final product in the 12-step calculated route (FIG. 31) to (+)-waihoensene, featuring a Michael addition—alkylation and a [4+2]cycloaddition as key steps. FIG. 32B: Graph edit distance analysis of the calculated route.

FIG. 33 shows total synthesis of (+)-welwitindolinone A from carvone oxide and atom mapping of the route.

FIGS. 34A-34B show: FIG. 34A: Adjacency matrix analysis of each starting material set, intermediate and final product in the 6-step synthesis of (+)-welwitindolinone A from carvone oxide, featuring a cascade reaction of an indole fluorination—trapping with water—elimination of fluoride—[1,5] sigmatropic rearrangement as a key step. FIG. 34B: Graph edit distance analysis of the route.

FIG. 35. Total synthesis of (+)-frondosin B and atom mapping of the route.

FIGS. 36A-36B show: FIG. 36A: Adjacency matrix analysis of each starting material set, intermediate and final product in the 17-step synthesis of (+)-frondosin B, featuring a Diels-Alder reaction of a sterically demanding diene and nitroethylene as a key step. FIG. 36B: Graph edit distance analysis of the route.

FIG. 37 is the total synthesis of (+)-englerin A and atom mapping of the route.

FIGS. 38A-38B show: FIG. 38A: Adjacency matrix analysis of each starting material set, intermediate and final product in the 15-step synthesis of (+)-englerin A, featuring a ring-closing olefin metathesis as a key step. FIG. 38B:Graph edit distance analysis of the route.

FIG. 39. Total synthesis of (−)-englerin A from a known 3-furanone and atom mapping of the route.

FIGS. 40A-40B show: FIG. 40A: Adjacency matrix analysis of each starting material set, intermediate and final product in the 6-step synthesis of (−)-englerin A from a known 3-furanone, featuring a diastereoselective Michael addition and a SmI2-mediated reductive carbonyl-alkene cyclization as key steps. FIG. 40B: Graph edit distance analysis of the route.

FIG. 41 is the total synthesis of (−)-strychnine and atom mapping of the route.

FIGS. 42A-42B show: FIG. 42A: Adjacency matrix analysis of each starting material set, intermediate and final product in the 12-step synthesis of (−)-strychnine, featuring an organocascade addition-cyclization and a cascade Jeffery-Heck cyclization/lactol formation sequence as key steps. FIG. 42B: Graph edit distance analysis of the route.

FIG. 43 is the total synthesis of (+)-strychnine and atom mapping of the route.

FIGS. 44A-44B show: FIG. 44A: Adjacency matrix analysis of each starting material set, intermediate and final product in the 9-step synthesis of (+)-strychnine (longest linear sequence of six steps), featuring a base-mediated bicyclization reaction of a Zincke aldehyde and a cascade of Brook rearrangement—intramolecular conjugate addition as key steps. FIG. 44B: Graph edit distance analysis of the route.

FIG. 45. is the total synthesis of (+)-stemoamide and atom mapping of the route.

FIGS. 46A-46B show: FIG. 46A: Adjacency matrix analysis of each starting material set, intermediate and final product in the 6-step synthesis of (+)-stemoamide, featuring an organocatalyzed asymmetric Mannich reaction—in situ allylation as a key step. FIG. 46B: Graph edit distance analysis of the route.

FIG. 47 is the total synthesis of (−)-stemoamide and atom mapping of the route.

FIGS. 48A-48B show: FIG. 48A: Adjacency matrix analysis of each starting material set, intermediate and final product in the 3-step synthesis of (−)-stemoamide in this work, featuring a Michael addition—alkylation and a Schmidt-Aubé rearrangement of a cyclobutanone intermediate as key steps. FIG. 48B: Graph edit distance analysis of the route.

FIG. 49 is the total synthesis of (+)-stemoamide with a new hypothetical anti-Markovnikov hydroamidation via the undercutting strategy and atom mapping of the route.

FIGS. 50A-50B show: FIG. 50A: Adjacency matrix analysis of each starting material set, intermediate and final product in the 4-step synthesis of (+)-stemoamide with a hypothetical anti-Markovnikov hydroamidation, identified by the undercutting strategy. It also features an organocatalyzed asymmetric Mannich reaction—in situ allylation as a key step. FIG. 50B: Graph edit distance analysis of the route.

FIGS. 51A-51C show: FIG. 51A: a synthetic route (+)-welwitindolinone A by Baran (Nature 446, 404-408 (2007)). FIG. 51B: maximum common substructure distance analysis and graph edit distance analysis of the route. FIG. 51C: analysis of the route by combining two methods.

### DETAILED DESCRIPTION

The present disclosure provides methods and systems for selecting chemical synthesis processes for efficient production of chemical compounds. In particular, disclosed herein is a strategy that merges high-throughput computer-aided synthesis planning with molecular graph editing to minimize the number of synthetic steps required to produce chemical compounds. In an exemplary process, an enantioselective 3-step synthesis of (−)-stemoamide and a 5-step synthesis of (−)-stemonine were developed by leveraging high-impact key steps, which could be identified and repurposed using graph edit distances.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise.

The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “computer memory” and “computer memory device” refer to any storage media readable by a computer processor. Examples of computer memory include, but are not limited to, RAM, ROM, computer chips, digital video discs (DVD), compact discs (CDs), hard disk drives (HDD), and magnetic tape.

As used herein, the term “computer readable medium” refers to any device or system for storing and providing information (e.g., data and instructions) to a computer processor.

Examples of computer readable media include, but are not limited to, DVDs, CDs, hard disk drives, magnetic tape, and servers for streaming media over networks. The computer readable medium may be non-transitory or include a device of system that is not a transitory signal.

As used herein, the terms “processor” and “central processing unit” or “CPU” are used interchangeably and refer to a device that is able to read a program from a computer memory (e.g., ROM, or other computer memory) and perform a set of steps according to the program.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Identifying Chemical Syntheses

The present disclosure provides methods for identifying efficient chemical synthesis processes using molecular graph editing.

In some embodiments, the disclosed methods are computational or computer-implemented methods that merge high-throughput computer-aided synthesis planning with molecular graph editing, to achieve a goal in the synthetic process for the production of chemical compounds, including complex natural products. Exemplary methods disclosed herein are for synthesis of alkaloids, but one skilled in the art will recognize that the methods can be used for identifying syntheses of other classes of chemical compounds.

The identified chemical synthesis processes can be designed such that there are fewer steps than previously developed or proposed synthesis processes for the target molecule. Alternatively or in addition, the identified chemical synthesis processes can be designed to control other desired outcomes for a new chemical synthesis process for the target molecule as compared to those previously known. For example, the identified chemical synthesis may decrease reagent costs, utilize available starting or intermediate compounds or reagents, and/or increase yield when compared to previously developed or proposed synthesis processes for the target molecule.

In some embodiments, the disclosed methods include determining graph edit distances for the target molecule and all synthetic intermediates for one or more routes of synthesis of a target molecule. Graph edit distances can be determined by a variety of methods, including those described here in the examples, and provide a similarity measurement between molecular structures, e.g., molecular structures of starting and ending compounds formed as a result of a chemical step or synthesis method. Thus, in some embodiments, the methods include any one, two, or all of: generating adjacency matrices for the target molecule and all synthetic intermediates for one or more routes of synthesis of the target molecule; determining graph edit distances from the adjacency matrices; and identifying a chemical synthesis process.

In some alternative or additional embodiments, the methods include determining maximum common substructure (MCS) distance for the target molecule and all synthetic intermediates. Thus, in some embodiments, the methods include any one, two, or all of: generating adjacency matrices for the target molecule and all synthetic intermediates for one or more routes of synthesis of the target molecule; determining maximum common substructure (MCS) distance for the target molecule and all synthetic intermediates; and identifying a chemical synthesis process. In some embodiments, the methods include any one, two, three, or all of: generating adjacency matrices for the target molecule and all synthetic intermediates for one or more routes of synthesis of the target molecule; determining graph edit distances from the adjacency matrices; determining maximum common substructure (MCS) distance for the target molecule and all synthetic intermediates; and identifying a chemical synthesis process.

The MCS distance takes into account the largest collection of atoms that can be completely overlaid on the target. Thus, similar to the graph edit distances, MCS distances also provide similarity measurement between molecular structures but does so based on matching atom identity with the target compound. Several methods are possible for generation of MCS distances; exemplary methods are included in the Examples.

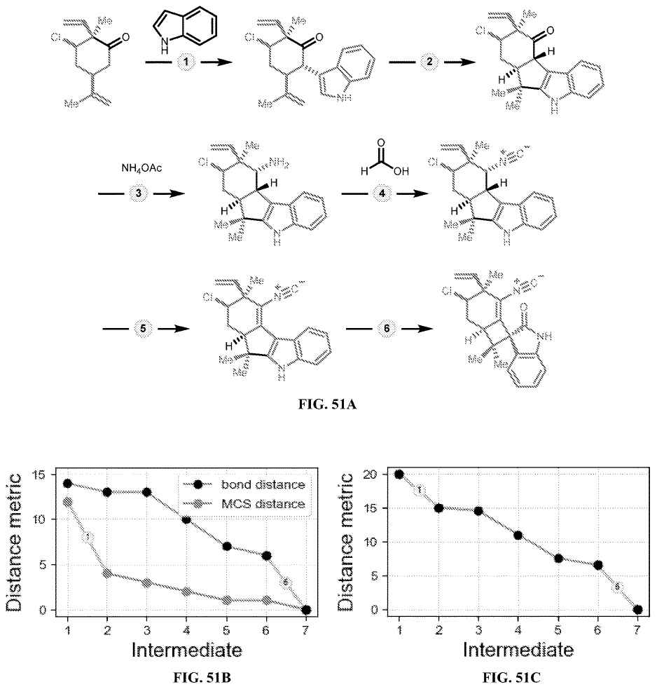

In some embodiments, the methods further include calculating a final distance metric based on the maximum common substructure (MCS) distance and the graph edit distances. Thus, the final distance metric accounts for steps that are impactful through adding many target atoms, as well as steps that add many target bonds. See for example FIG. 51 and Example 1. Accordingly, in some embodiments, the methods include any one, two, three, four, or all of: generating adjacency matrices for the target molecule and all synthetic intermediates for one or more routes of synthesis of the target molecule; determining graph edit distances from the adjacency matrices; determining maximum common substructure (MCS) distance for the target molecule and all synthetic intermediates; calculating a final distance metric; and identifying a chemical synthesis process.

The one or more (e.g., two or more, three or more, four or more, five or more, etc.) routes of synthesis and the structures of synthetic intermediates can be derived from previously-developed synthesis processes for the target molecule, e.g., those experimentally determined, such as those disclosed in chemical literature, those generated by standard computer aided synthesis planning (CASP) methods, such as SYNTHIA™, or a combination thereof. In some embodiments, the CASP methods can be automated retrosyntheses. The CASP methods can be focused with reaction rules for any desired outcome, e.g., minimization of protecting group manipulations, maximize convergency, maximum reliability of reaction, and the like. Accordingly, in some embodiments, the methods further comprise generating one or more (e.g., two or more, three or more, four or more, five or more, etc.) CASP methods for a desired target molecule, e.g., for use in the disclosed methods.

In some embodiments, the methods include a step of generating adjacency matrices for the target molecule and all synthetic intermediates. Adjacency matrices are generated in which the number of rows and columns is equal to the total number of heavy atoms and groups used in the entire synthetic route. In this way, all bonds of the final target and their progression from starting materials, as well as any concession groups used in the synthesis, are mapped exactly in each individual matrix and in relation to the final target's matrix. Concession bonds are those that are broken during the synthesis process, and concession atoms are those that do not appear in the final target compound. Concession groups (collections of atoms that do not undergo any transformation throughout the synthetic route) can be encoded as a single atom without losing information relevant to the results presented herein (e.g., alkyl side chains and protecting groups). Each adjacency matrix is an (Nₜ+N_{c})×(Nₜ+N_{c}) square, where Nₜ is the number of atoms and groups in the target molecule, and N_{c} is the number of concession atoms and groups. Several methods are possible for generation of these matrices; exemplary methods are included in the Examples. Distances, as described herein, can be determined from the adjacency matrices. Methods for computing such distances are disclosed in the Examples.

In some embodiments, the methods comprise identifying one or more high impact steps in the one or more routes of synthesis. High impact reaction steps have a steep slope on distance plot (e.g., graph edit distance, MCS distance, or final distance metric plots) between given intermediates en route to the final target. In some embodiments, the high impact reaction step has a steeper slope relative to other steps in the route of synthesis. High impact steps based on graph edit distance maximize the change in graph edit distance from a given intermediate to the target, which is equivalent to maximizing formation of target bonds, while minimizing reaction manipulations on concession groups. Thus, in some embodiments, the high impact steps form two or more requisite target bonds simultaneously. High impact steps based on MCS distance have the greatest contribution to lowering MCS distance. Thus, in some embodiments, the high impact steps result in a large increase in the atoms, which can be completely overlaid on the target. Overall, high impact steps are those which move an intermediate in the process, in atom identity and/or bond order, closer to the target compound.

In some embodiments, many different known or proposed synthetic routes to a target compound can be analyzed to search for steps that can be incorporated into the final synthesis process. Uniting multiple high impact steps from diverse synthetic routes can ultimately lead to concise new synthesis methods. Thus, the identified chemical synthesis process may combine high impact steps from two or more different routes of synthesis, experimental or synthetic, for the target molecule.

In some embodiments, the methods further include identifying modest to low impact steps in the one or more routes of synthesis. The modest to low impact steps are those which have a shallow or shallower slope on distance plot (e.g., graph edit distance, MCS distance, or final distance metric plots) between given intermediates en route to the final target. In other words, modest to low impact steps result in few changes, in atom identity and/or bond order, to move an intermediate closer to the structure of the target compound. Modest to low impact steps can include, for example, transformations or manipulations on concession groups as described above. Examples of low impact steps could include the installation/removal of protecting groups, unnecessary redox manipulations, and functional group interconversions. In general, a low impact step installs few if any of the bonds or atoms required for the final target molecule.

In some embodiments, the methods further include grouping the neighboring modest to low impact steps into fewer steps with higher impact. Thus, users can evaluate identified routes to determine whether shortcuts can be developed. For example, neighboring modest to low impact steps may be combined into a single high impact step, resulting in the identified chemical synthesis process having a minimal number of modest to low impact steps.

Overall, the disclosed methods measure step impact of one or more routes of synthesis of the target molecule and maximize high impact steps while minimizing low impact steps.

The methods described herein can be implemented in digital electronic circuitry, or in computer software, firmware, or hardware. The methods can be implemented as one or more computer programs, e.g., one or more modules of computer program instructions, encoded on computer storage medium for execution by, or to control the operation of, data processing apparatus. Alternatively, or in addition, the program instructions can be encoded on an artificially generated, propagated signal, for example, a machine-generated electrical, optical, or electromagnetic signal that is generated to encode information for transmission to suitable receiver apparatus for execution by a data processing apparatus. In some embodiments, the methods are implemented as a non-transitory computer-readable medium storing instructions executable by one or more processors to perform operations.

As such, the present disclosure also provides non-transitory computer-readable media. The non-transitory computer-readable media stores instructions that when executed by one or more processors performs some or all of the operations described in the disclosed methods.

In some embodiments, the one or more processors perform operations including at least one or all of: generating adjacency matrices for the target molecule and all synthetic intermediates for one or more routes of synthesis of the target molecule; determining graph edit distances from the adjacency matrices; and identifying a chemical synthesis process. In some embodiments, the processors also perform the operations of: determining maximum common substructure (MCS) distance; and calculating a final distance metric based on the maximum common substructure (MCS) distance and the graph edit distances.

In some embodiments, the processors may further perform any or all of the operations of: identifying one or more high impact steps in the one or more routes of synthesis; identifying neighboring modest to low impact steps in the one or more routes of synthesis; and grouping the neighboring modest to low impact steps into fewer steps with higher impact.

In some embodiments, the processors may also perform the operations of: generating or receiving one or more routes of synthesis of the target molecule. For example, the processors may generate CASP routes of synthesis, may import CASP routes of synthesis from a CASP program, may import experimentally determined routes of synthesis, e.g., from chemical literature, or may receive user provided routes of synthesis of the target molecule.

The methods described herein can be implemented as a system including one or more processors and a computer-readable medium storing instructions executable by the one or more processors to perform operations, as described above. The system may comprise at least one computer system comprising the one or more processors and/or the computer-readable media. The system may further comprise one or more local servers or databases connected to or integrated with the one or more computer system. The one or more processors may be configured to communicate via wired or wireless communications with each other or other processors. The one or more processors may be configured to operate on one or more processor-controlled devices that can be similar or different devices.

### EXAMPLES

### Example 1: Methods for Adjacency Matrix Generation and Graph Edit Distance Analysis

All code was written and executed on a laptop running Microsoft Windows version 10.0.19044, python version 3.9.7. All dependencies are installed using conda (version 4.10.3). Versions of selected packages are as follows: ipython 7.27.0, jupyterlab 3.1.12, matplotlib 3.4.3, numpy 1.20.3, pandas 1.3.3, RDKit 2021.03.5.

### General Procedure 1: Generation of Matrix-Encoded Synthetic Routes

First, adjacency matrices for the target molecule and all synthetic intermediates were generated. Each resultant matrix is an (Nₜ+N_{c})×(Nₜ+N_{c}) square, where Nₜ is the number of atoms and groups in the target molecule, and N_{c} is the number of concession atoms and groups. A group refers to a collection of atoms that do not undergo any transformation throughout the synthetic route, and hence can be encoded as a single atom without losing information relevant to the results presented herein. Examples include alkyl side chains and protecting groups.

Several methods are possible for the generation of these matrices. The process employed herein is as follows:
1. Encode the product as a mol object in RDKit, replacing large unchanged substituents with single atoms as desired.
2. Iterate over the product's atoms using GetAtomso, and extract their atomic number using GetAtomicNum( ). Should the mol object be encoded directly from a SMILES string, the order in which atoms appear in GetAtomso will be the same as that in the SMILES string.
3. Use GetAdjacencyMatrix( ), setting useBO=True to generate the product adjacency matrix from the mol object. The ordering of product atoms along the rows and columns will be the same as that extracted in step 2.
4. Working backwards from the product, note down the following for every reverse step:
   a. The number and identities of additional concession atoms to be appended.
   b. Atom indices at which there is a change in bond order.
   c. Changes in symmetry about atoms that are asymmetric in the product.

These changes are transferred to a bond edit spreadsheet. An example of a short spreadsheet is depicted in Table 1.

TABLE 1
Sample bond edit spreadsheet, with which the
matrix-encoded synthetic route is generated

| bond | Edit | file |
|---|---|---|
| step | Addstereo | 16 |
| pad | 0 | 0 |
| 3 | 3 | 1 |
| 6 | 6 | 1 |
| 7 | 7 | 1 |
| 8 | 8 | 1 |
| step | Lactam | 16 |
| pad | 1 | 8 |
| 11 | 12 | −1 |
| 11 | 17 | 1 |
| step | ringAmination | 17 |
| pad | 1 | 17 |
| 12 | 8 | −1 |
| 8 | 18 | 1 |
| step | End | end |

These changes are transferred to a bond edit spreadsheet. An example of a short spreadsheet is depicted in Table 1.

Each retrosynthetic step is split into three sections:
1. The “step” row, which separates individual steps. The cells highlighted in green are not read by the matrix generation algorithm, and are intended as flexible annotation spaces for the user. In the files for this disclosure, they are most commonly used to keep track of step names and maximum atom count.
2. The “pad” row, whose first entry is the number of concessions atoms to add, and the second is their atomic numbers, in order, separated by spaces.
3. The bond edit block, where, for each row, the first two entries are indices of the atoms joined by a changing bond, and the third is the change in bond order.

After the final step is encoded (e.g., the first step in the forward synthesis), a last row is added to mark the end of the bond edit file.

Most edit files for this disclosure use the first “step” to add in stereocenters as diagonal entries. R and S stereocenters are not distinguished in this work, and only encoded as 1 for present, and 0 for absent. Stereocenters that appearing during the synthesis, but are absent in the final product, are not encoded.

After the bond edit file is complete, it is used to compute and save the adjacency matrices for the remaining intermediates. Should any changes be difficult to encode via a bond edit spreadsheet, they can be added to the matrices after they are generated.

### General Procedure 2: Computation of Graph Edit Distances

All matrix-encoded synthetic routes are saved as .npz files containing two arrays:
amats, a r×(Nₜ+N_{c})×(Nₜ+N_{c}) array, where r is the number of intermediates in the synthetic route, inclusive of the product, and
atoms, a (Nₜ+N_{c})×1 array containing the atomic numbers, corresponding to the rows of amats.

When amats is iterated over, it generates a series of intermediate matrices, M₁ . . . Mᵢ . . . M_{P}, where Mᵢ is the adjacency matrix for intermediate i, and M_{P} is that of the product.

For each intermediate matrix Mᵢ, its graph edit distance from M_{P}, D_{i,P} is computed as follows:
1. Mᵢ is split into two sections: Bᵢ, which encodes bond order, and Sᵢ, which encodes stereochemistry. The same is done for M_{P}.
2. Compute R_{i,P} the reaction matrix that brings Bᵢ to B_{P}, by performing B_{P}−Bᵢ.
3. Compute bond edit distance between Bᵢ and B_{P} by summing up all elements of abs(R_{i,P}) and dividing by 2.
4. Compute stereochemical distance with Σ(S_{P}−Sᵢ), where Σ denotes summing over all array elements.
5. The graph edit distance, D_{i,P}, is the sum of bond edit distance and stereochemical distance.

Graph edit distance plots are generated by plotting D_{i,P} against i, and the slope of step j computed by D_{j,P}−D_{j-1,P}.

### Evaluating and Plotting Adjacency Matrices Based on Bond Type

1. All adjacency matrices Mᵢ are iterated over. For every matrix, all non-zero entries in its lower triangle are collected as tuples of (m, n), where m and n are atom indices at which a bond exists. That is, for every (m, n) pair, there exists at least one value of i for which M_{i,m,n}>0.
2. For each (m, n) pair, M_{i,m,n} is evaluated for each value of i. In terms of the molecular system, the bond that joins atoms m and n is searched for in every intermediate. This generates the bond history array, denoted as H_{m,n}, an r×1 array that indicates the bond order between atoms m and n through all intermediates in the synthetic route.
3. The type of every bond can be evaluated by considering the elements of H_{m,n}:
   a. For purchased bonds, all entries of H_{m,n} are positive, indicating a bond that is present in the starting system, and persists through to the product.
   b. For strategic bonds, the first entry of H_{m,n}=0, but the last is positive, indicating
   a bond that is absent in the starting system, but present in the product.
   c. For concession bonds, the last entry of H_{m,n}=0, but some prior entries are positive, indicating a bond that is present during synthesis, but absent in the product itself. An exception is made if both m and n are greater than Nₜ, the number of atoms and groups in the target molecule. This corresponds to a bond joining two concession atoms, such as the 33-34 bond in a Frondosin B synthetic route (Inoue et al. J. Am. Chem. Soc. 123, 1878-1889 (2001)). These are counted as concession bonds, even if they are present in the product.
4. As the type of each bond is evaluated, the indices of the atoms it joins are sorted into three categories, each corresponding to one type described in step 3. These are used to color entries in the adjacency matrix plots accordingly.

### Evaluation of Molecular Fingerprint-Based Similarity Metrics

1. The matrix-encoded synthetic route is read and converted to mol objects using RDKit, one corresponding to each matrix. As the entire molecular system is represented in every matrix, all mol objects will contain more than one molecule.
2. Using GetMorganFingerprintAsBitVect( ), 2048-bit Morgan fingerprints with radius 2 were obtained for each intermediate.
3. The Morgan fingerprints are iterated through, computing selected distances from each intermediate to the final product. Six similarity metrics were computed: Tanimoto, Cosine, McConnaughey, Dice, Russel and Kulczynski, and the first three were plotted to show a wide range of meaningful, non-overlapping trends.

### General Procedure 3: Generation of Maximum Common Substructure (MCS) Distance

1. Encode all compounds as mol objects in RDKit.
2. Use rdFMCS.FindMCS( ) to find the maximum common structure between each intermediate and the target.
3. Use mcs.numAtoms to extract the number of common atoms between intermediate and the target.
4. To get the MCS distance, subtract the number of common atoms of each intermediate from the number of atoms in the final target.

### Example 2: Synthesis

All reactions were conducted in oven- or flame-dried glassware under an atmosphere of nitrogen unless stated otherwise. Reactions were set up in an MBraun LABmaster Pro Glove Box (H₂O level <0.1 ppm, 02 level <0.1 ppm), or using standard Schlenk technique with a glass vacuum manifold connected to an inlet of dry nitrogen gas. Solvents (acetonitrile (MeCN), tetrahydrofuran (THF), dichloromethane, toluene) were purified using a MBraun SPS solvent purification system, by purging with nitrogen, and then passing the solvent through a column of activated alumina. 1,4-Dioxane, N,N-dimethylformamide (DMF), dimethyl sulfoxide (DMSO), ethanol (EtOH) and 1,2-dichloroethane (DCE) were purchased as anhydrous solvents and used as received. Reagents [zinc powder, allyl bromide, 4-methoxyaniline, L-proline, DL-proline, (S,S)-DACH-phenyl Trost ligand, bismuth(III) chloride, trifluoroacetic acid (TFA), sodium hydroxide (NaOH), bis(1,5-cyclooctadiene)diiridium(I) dichloride ([Ir(cod)Cl]2), 1,2-bis(diphenylphosphaneyl)ethane (dppe), 4,4,5,5-tetramethyl-1,3,2-dioxaborolane (HBpin), hydrogen peroxide (H₂O₂), tetrabromomethane, triphenylphosphine, ceric ammonium nitrate (CAN), sodium hydride (NaH), tetrabutylammonium iodide, lithium bis(trimethylsilyl)amide (LiHMDS), methyl iodide (Mel), acetic acid, 5-hydroxyfuran-2(5H)-one, n-butyllithium (2.5 M in hexanes, n-BuLi), 2,2,2′-trimethylpropionanilide, (S)-(−)-1-amino-2-(methoxymethyl)pyrrolidine (SAMP), titanium(IV) chloride (TiCl₄), (+)-Ipc₂B(allyl)borane (1 M in pentane), cyclobutanone, 1-hydroxy-1,2-benziodoxol-3(1H)-one, azidotrimethylsilane, 2,2,2′-trimethylpropionanilide, rhodium on alumina (5 wt %), Vaska's complex IrCl(CO)(PPh₃)₂, 1,1,3,3-tetramethyldisiloxane (TMDS), 2-nitrobenzoic acid, triisopropylsilyltriflate, 3-methylfuran-2(5H)-one, triethylamine, tris(dibenzylideneacetone)dipalladium(0) (Pd₂dba₃), tris(dibenzylideneacetone)dipalladium(0)-chloroform adduct (Pd₂dba₃·CHCl₃), allylpalladium(II) chloride dimer ([Pd(allyl)Cl]₂), allyl acetate, allyl methyl carbonate, 2-(trimethylsiloxy)furan]were purchased from MilliporeSigma, Alfa Aesar, Ambeed, Strem, or TCI Chemical. All chemicals were used as received. n-BuLi was titrated with 2,2,2′-trimethylpropionanilide prior to use. Methyl 4-oxobutanoate (7, 8), was prepared according to literature procedures, and its published characterization data matched with our own in all aspects. Glass 2-dram vials (ChemGlass #CG-4912-02) were used as reaction vessels, fitted with a screw-cap, Teflon-coated silicone septum (ChemGlass #CG-4910-02), and magnetic stir bars (Fisher Scientific #14-513-93 or #14-513-65). Proton nuclear magnetic resonance spectra (¹H NMR) were recorded on a Varian MR-700 MHz, Varian MR-500 MHz, or Varian MR-400 MHz spectrometer and chemical shifts are reported in parts per million (ppm) using the solvent residual peak as an internal standard (CDCl₃ at 7.26 ppm, DMSO-d₆ at 2.50 ppm). Data are reported using the abbreviations: app=apparent, s=singlet, d=doublet, t=triplet, q=quartet, quint=quintuplet, sext=sextuplet, sept=septuplet, m=multiplet, comp=complex, br=broad. Coupling constant(s) “J” are reported in Hz. Proton-decoupled carbon nuclear magnetic resonance spectra (¹³C NMR) spectra were recorded on a Varian MR-700 MHz, Varian MR-500 MHz or Varian MR-400 MHz spectrometer and chemical shifts are reported in ppm using the solvent as an internal standard (CDCl₃ at 77.16 ppm, DMSO-d₆ at 39.52 ppm). High resolution mass spectrometry data (HRMS) was obtained on an Agilent 6230 TOF LC/MS equipped with ESI detector in positive mode. The enantiomeric excesses were determined by SFC analysis on a Waters Investigator supercritical fluid chromatography (SFC) system employing a chiral stationary phase column and conditions specified in the individual experiments. Optical rotations were measured at room temperature in a solvent of choice on a JASCO P-2000 digital polarimeter at 589 nm (D-line). Reaction analysis was typically performed by thin-layer chromatography on silica gel, or using a Waters I-class ACQUITY UPLC-MS (Waters Corporation, Milford, MA, USA) equipped with in-line photodiode array detector (PDA) and QDa mass detector (ESI positive ionization mode). 0.1 μL sample injections were taken from acetonitrile solutions of reaction mixtures or products (˜1 mg/mL). A partial loop injection mode was used with the needle placement at 1.0 mm from bottom of the wells and a 0.2 μL air gap at pre-aspiration and post-aspiration. Column used: Waters Cortecs UPLC C18+ column, 2.1 mm×50 mm with (Waters #186007114) with Waters Cortecs UPLC C18+ VanGuard Pre-column 2.1 mm×5 mm (Waters #186007125), Mobile Phase A: 0.1% formic acid in Optima LC/MS-grade water, Mobile Phase B: 0.1% formic acid in Optima LC/MS-grade MeCN. Flow rate: 0.8 mL/min. Column temperature: 45° C. The PDA sampling rate was 20 points/sec. The QDa detector monitored m/z 150-750 with a scan time of 0.06 seconds and a cone voltage of 30 V. The PDA detector range was between 210 nm-400 nm with a resolution of 1.2 nm. 1-minute and 2-minute methods were used. The method gradients are below: 0 min: 0.8 mL/min, 95% 0.10% formic acid in water/5% 0.10% formic acid in acetonitrile; 1.5 min: 0.8 mL/min, 0.10% 0.10% formic acid in water/99.9% 0.10% formic acid in acetonitrile; 1.91 min: 0.8 mL/min, 95% 0.1% formic acid in water/5% 0.1% formic acid in acetonitrile. Flash chromatography was performed on silica gel (230-400 Mesh, Grade 60) under a positive pressure of nitrogen. Thin layer chromatography was performed on 25 μm TLC Silica gel 60 F₂₅₄ glass plates (Fisher Scientific #S07876). Visualization was performed using ultraviolet light (254 nm) and potassium permanganate (KMnO₄) stain.

### Selection of Synthesis Processes for (−)-Stemoamide and (−)-Stemonine

Stemoamide (1), a compound isolated from Stemonaceae plants, was selected as a target molecule because its four stereocenters and fused-ring structure would challenge modern CASP, and because it has been reported in thirty-two historic and contemporary syntheses ((Brito et al. Org. Prep. Proced. Int. 50, 245-259 (2018); Yoritate et al., J. Am. Chem. Soc. 139, 18386-18391 (2017); Yin et al., Org. Lett. 22, 5001-5004 (2020); Siitonen et al., Synlett 31, 1581-1586 (2020); Guo et al., Angew. Chem. Int. Ed. 60, 14545-14553 (2021); Cao et al., Org. Lett., 23, 6222-6226 (2021); Shi et al., Org. Chem. Front. 9, 771-774 (2022); Wang et al., Org. Chem. Front. 9, 3818-3822 (2022); Rosso et al., Eur. J. Org. Chem. 2022, e202200585 (2022)), providing a strong benchmark for comparison. Stemoamide and its congeners are also keystone intermediates en route to many other members of the stemona alkaloid family.

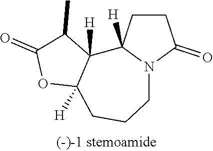 [SMILES: C[C@@H]1C(=O)O[C@@H]2CCCN3C(=O)CC[C@H]3[C@@H]12]

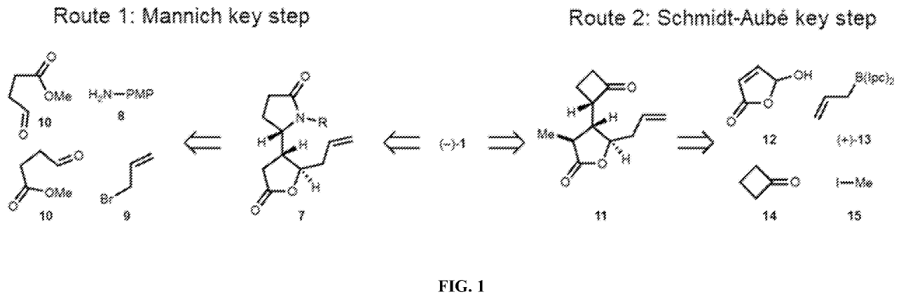

Two distinct retrosynthetic plans devised through CASP and graph editing, as described in detail below, are shown in FIG. 1. The first retrosynthetic plan removes the C10-methyl group and breaks the C—N bond of the azepine ring to give 7. The cyclized alkene 7 was itself produced by a key organocatalyzed Mannich-allylation-lactonization sequence recommended by CASP. Advanced intermediate 7 was further simplified to starting materials 8, 9 and two equivalents of aldehyde 10. A second route resulted from the evolution of a CASP and graph editing strategy, and was applied to both 1 and 2, the latter of which can be used to produce stemona alkaloids in the C10-ethyl series such as 5. In this route, a Schmidt-Aubé rearrangement recommended by CASP is a key simplifying element. The resultant cyclobutanone intermediate 11 was further reduced to 12, 13, 14, and 15 leveraging a key Michael addition and allylation sequence, which was identified as a key step in a separate CASP search of 3.

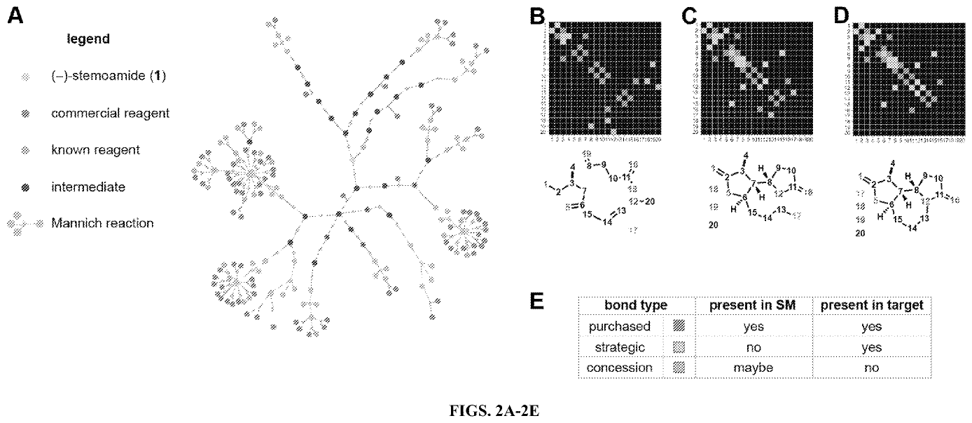

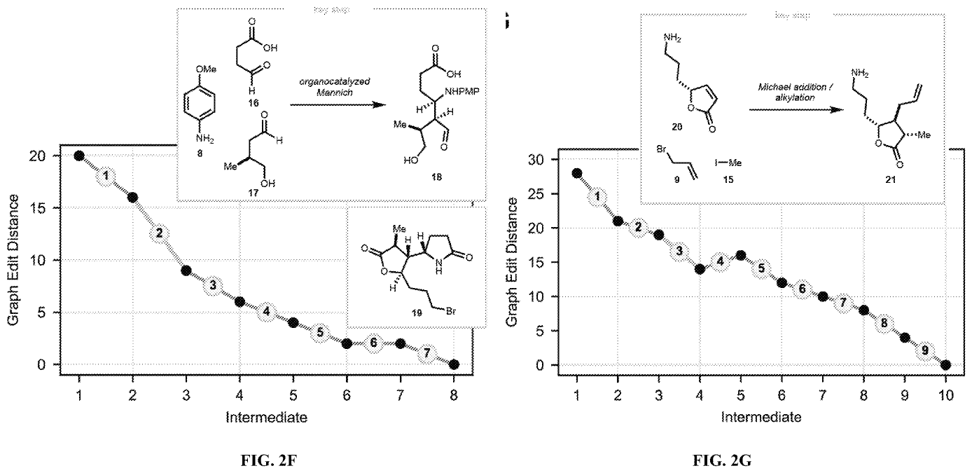

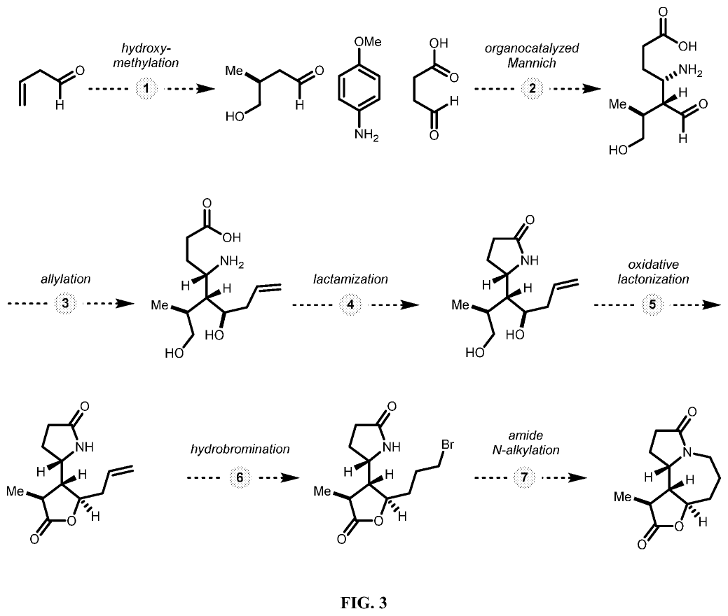

As a first attempt to minimize step count from CASP-generated routes, (−)-1 was subjected to automated retrosynthesis in the software SYNTHIA™ using a scoring function that promoted chemoselectivity, regioselectivity and stereoselectivity and demoted the use of protecting groups. An organocatalyzed Mannich reaction appeared as a proposed transformation in every predicted route (FIG. 2A). This was surprising since this well-known reaction had not yet featured in any of the 32 prior syntheses of 1. Nonetheless, even the shortest calculated route to 1 was 7 steps long (FIG. 3), which was competitive with, but not better than, the shortest human-derived enantioselective route (Yoritate et al., J. Am. Chem. Soc. 139, 18386-18391 (2017)). Calculated routes were thus logically edited for brevity by maximizing high impact transformations and minimizing low impact transformations. This required a new system to measure step impact.

Obvious inefficiencies such as protecting group manipulations could be minimized by the CASP software in the calculated routes, but it was unclear whether other specific steps among the hundreds of calculated retrosynthetic routes analyzed were impactful or not. In principal, the impact of a given synthetic step in a multistep sequence can be measured via the reduction in graph edit distance (Sanfeliu et al. IEEE Transactions on Systems, Man, and Cybernetics SMC-13, 353-362 (1983)) similarity metric between molecular graphs (David et al. J. Cheminform. 12, 56 (2020)) of intermediates and the target molecule. Efficient multistep synthesis converts the bonds and atoms of commercially available starting materials into the bonds and atoms of the target with key steps assembling a large portion of the target bonds and stereocenters simultaneously. Thus, high impact reaction steps should have a steep slope in a molecular graph edit distance plot between given intermediates en route to the final target.

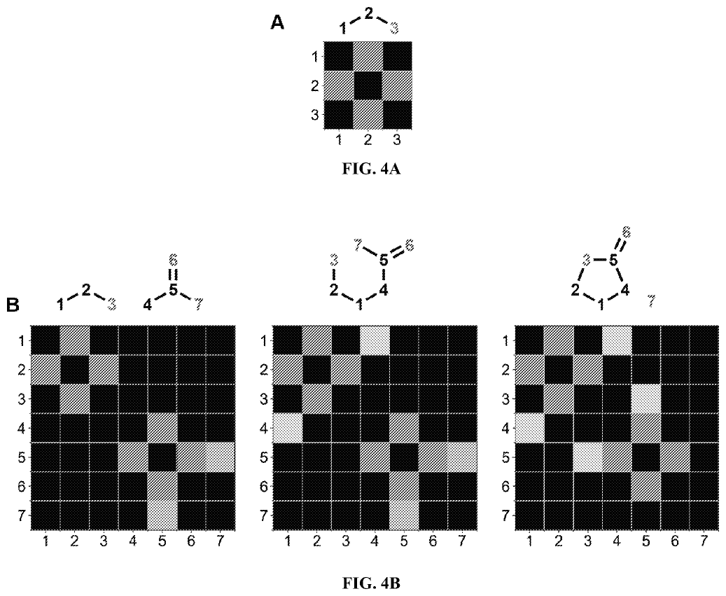

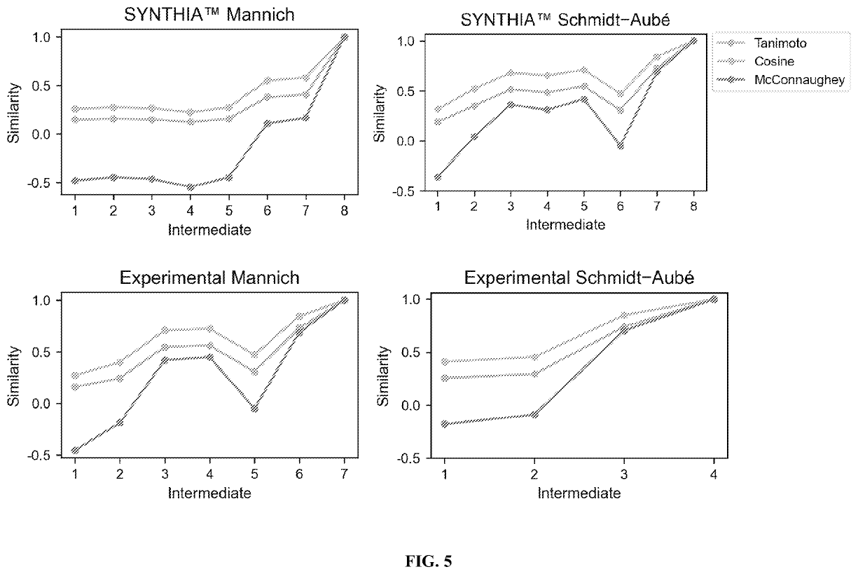

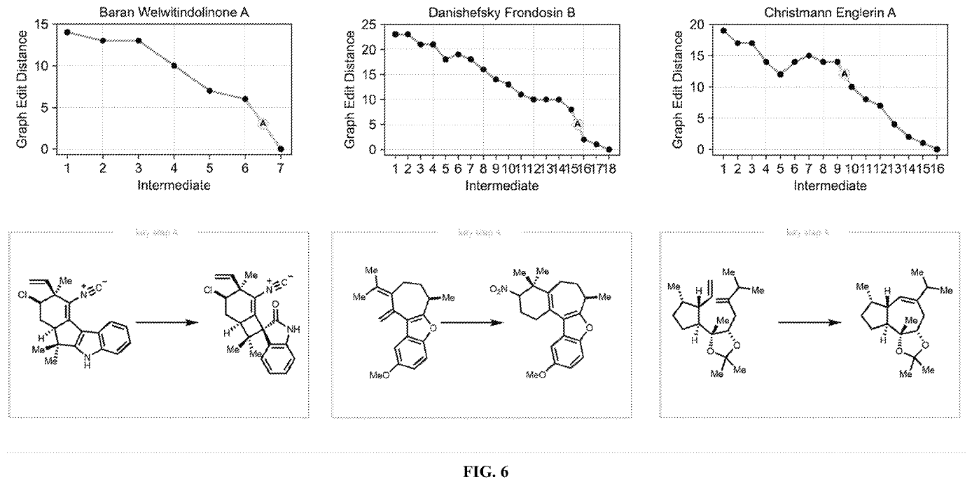

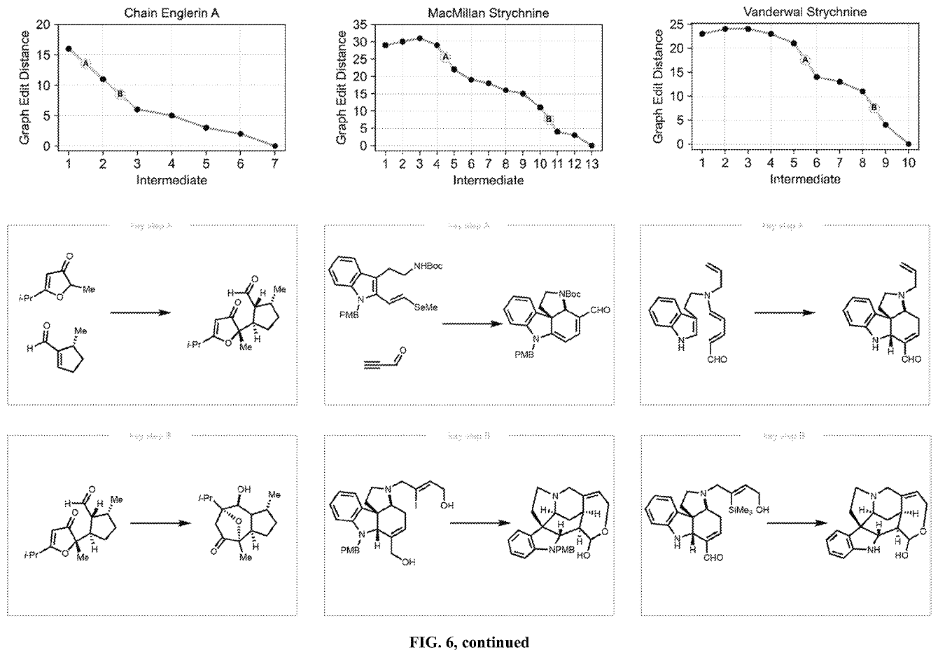

Molecular graphs of each intermediate were visualized, including starting materials and the final target, as individual adjacency matrices (FIGS. 2B-2D and FIG. 4) where the number of rows and columns is equal to the total number of heavy atoms and groups used in the entire synthetic route. In this way, all bonds of the final target and their progression from starting materials, as well as any concession groups used in the synthesis, are mapped exactly in each individual matrix and in relation to the final target's matrix (FIG. 2E). A simple comparison of the matrix for 1 (FIG. 2D) reveals it shares more similarity (99%) with the calculated penultimate intermediate 19 (FIG. 2C), than it does with the matrix for starting materials 8, 16, 17, 13 and HBr (FIG. 2B), which has 93% similarity to 1. Accordingly, key steps maximize the change in graph edit distance from a given intermediate to the target, which is equivalent to maximizing formation of target bonds, while minimizing reaction manipulations on concession groups. This graph formalization shares similarity with established concepts of synthetic ideality, but is machine readable, carries exact atom and bond mapping, and requires no labeling of reaction type to separate low impact reactions such as redox manipulations from impactful cycloadditions or cascade reactions. Since graphs capture the exact location of every bond, graph edit distance was superior to other metrics, such as Tanimoto distance based on Morgan fingerprints (FIG. 5), at highlighting the impact of key transformations. A survey of published total syntheses by graph edit distance (FIG. 6) shows that diverse key steps are readily visualized.

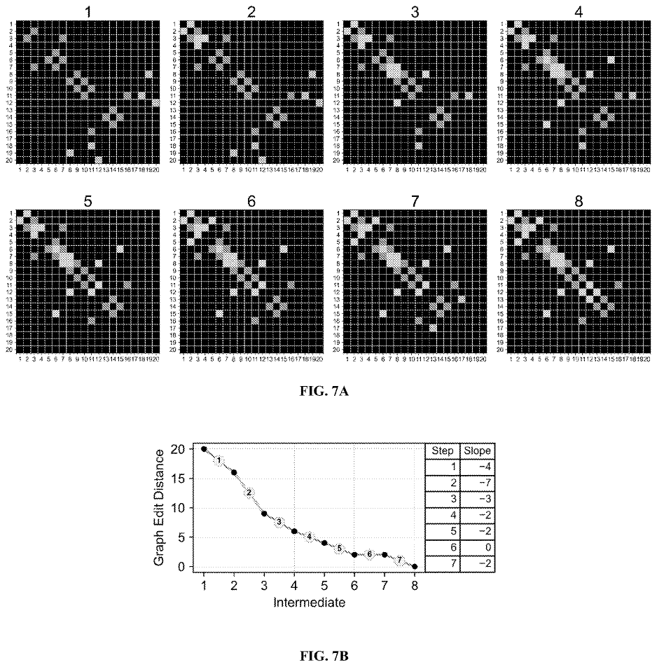

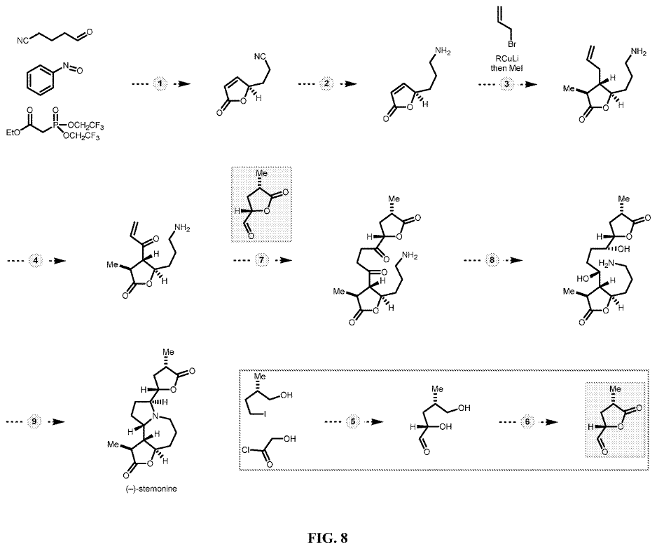

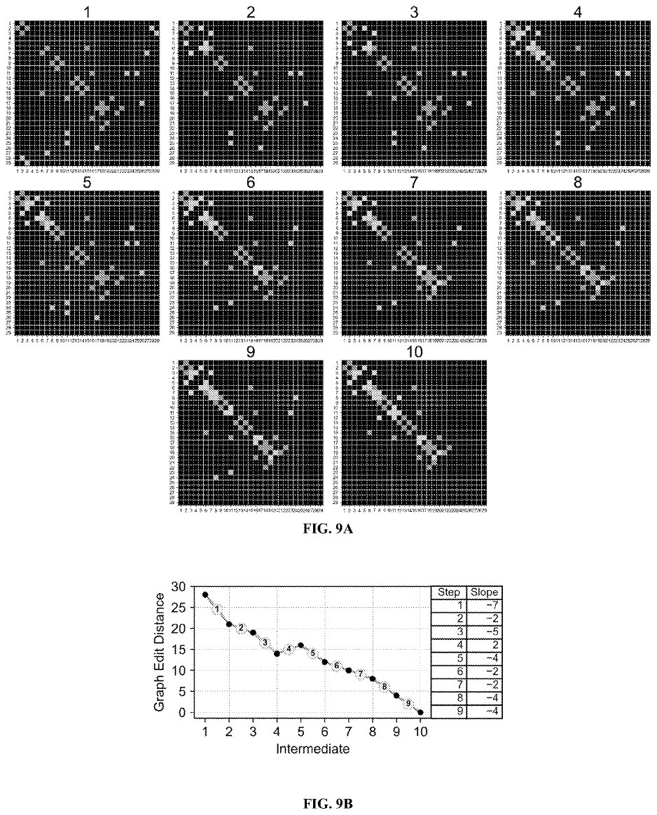

A full graph analysis of the shortest calculated route to 1 (FIGS. 7A-7B) reveals the impact of the Mannich coupling (FIG. 2F), which appears as the steepest declining step (yellow) in the graph edit distance plot. As another example, a survey of 50 calculated routes towards 3 revealed one route (FIG. 8) with several notable steps including a three-component coupling of 9, 20 and 15 by an impactful Michael Addition—alkylation sequence (FIG. 2G and FIG. 9).

While the computed routes to 1 focused attention on the Mannich disconnection (FIG. 2A) as an impactful key step, there were opportunities for improvement. For instance, C2 and C11 are both in the carboxyl oxidation state in 1, so by considering redox economy, the oxidation state of 16 and 17 could be harmonized to cut out two calculated steps. This realization unveiled a hidden symmetry element within 1, where two equivalents of commercially available aldehyde 10 (FIG. 3A) unite in a self-Mannich reaction. This would require installation of the C10 methyl group at a later stage, and fortuitously a diastereoselective C10-methylation had already been reported as a viable final step in several syntheses of 1 (Brito 2018; Yoritate 2017).

In experimental practice (FIG. 3A), stirring 8 with a four-fold excess of aldehyde 10 and 20 mol % L-proline in N,N-dimethylformamide at −15° C., then adding a mixture of allyl bromide, zinc, and bismuth chloride directly to the reaction vessel and warming to room temperature, cleanly delivered lactone 23 as the major product via intermediacy of Mannich-adduct 22. Rather than isolate 23, excess zinc and insoluble materials were removed from the reaction mixture by filtration, and the crude filtrate was treated with trifluoroacetic acid to provide lactam 24, which upon purification by silica gel chromatography was obtained in 38:1 diastereomeric ratio (dr), and 33% overall yield from 8. The desired product 24 was isolated in 99% enantiomeric excess (ee). Thus, five bonds, two rings and three stereocenters were rapidly formed in high selectivity via a two-step sequence requiring just one chromatographic purification. Next it was necessary to perform the hydrobromination of 24. The computed suggestion to use hydrobromic acid led to an intractable mixture. The best identified experimental protocol involved conversion of alkene 24 to primary alcohol 25 followed by bromination and in situ removal of the p-methoxyphenyl group giving 26. Two final steps, based on known reactions (Brito 2018, Yoritate 2017), were required to form the azepane ring (27) and install the C10 methyl group via diastereoselective enolate allylation giving (+)-1. The synthesis was performed in six steps and required only four chromatographic purifications, marking the shortest enantioselective total synthesis of 1. For convenience, (+)-1 was made by using L-proline, although the software accurately recognized that D-proline would lead to (−)-1. The experimental route that arose through modifications of the calculated route was subjected to a graph edit analysis (FIG. 3B). Here the high impact of the Mannich-allylation key step is readily apparent, as is the low impact of the ensuing functional group interconversions required to complete the synthesis.

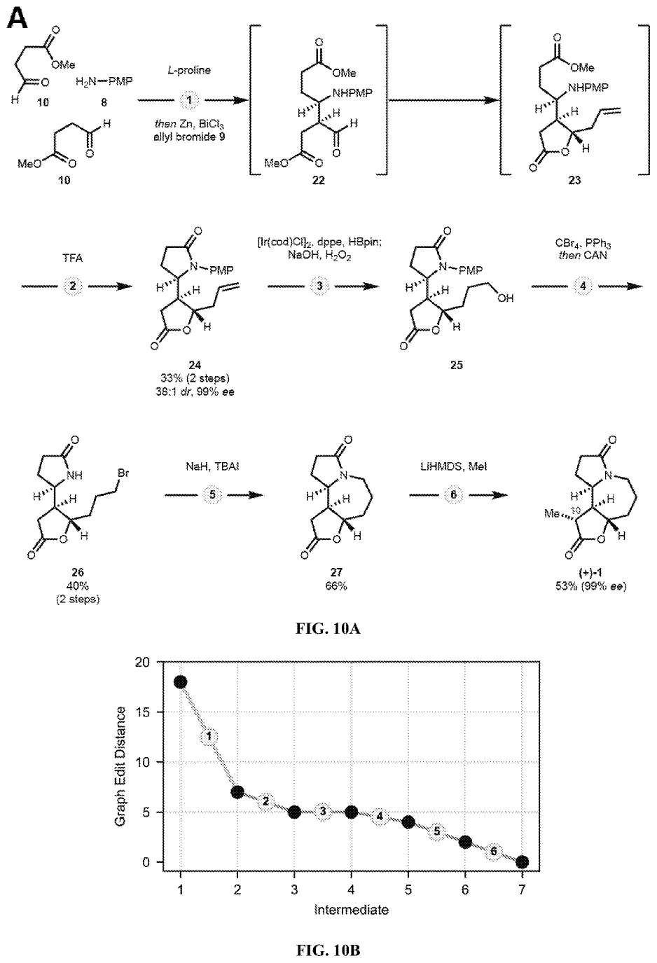

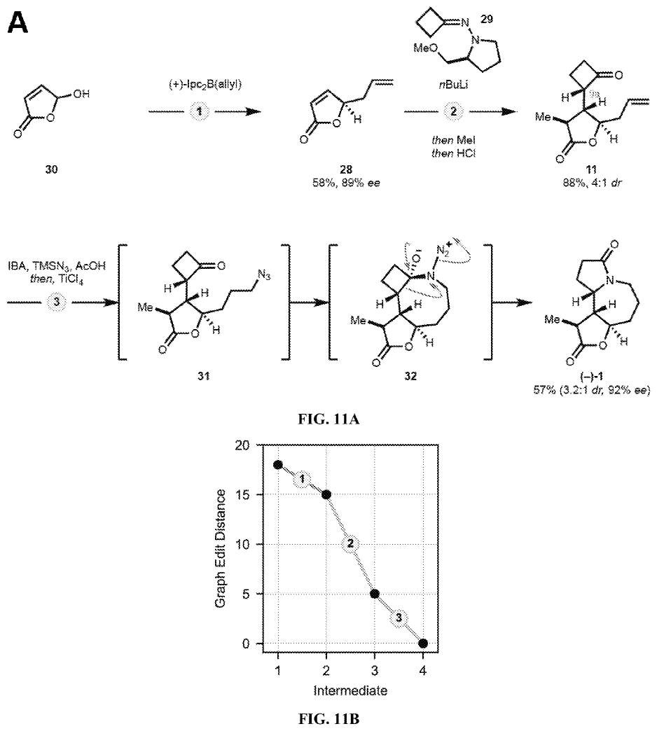

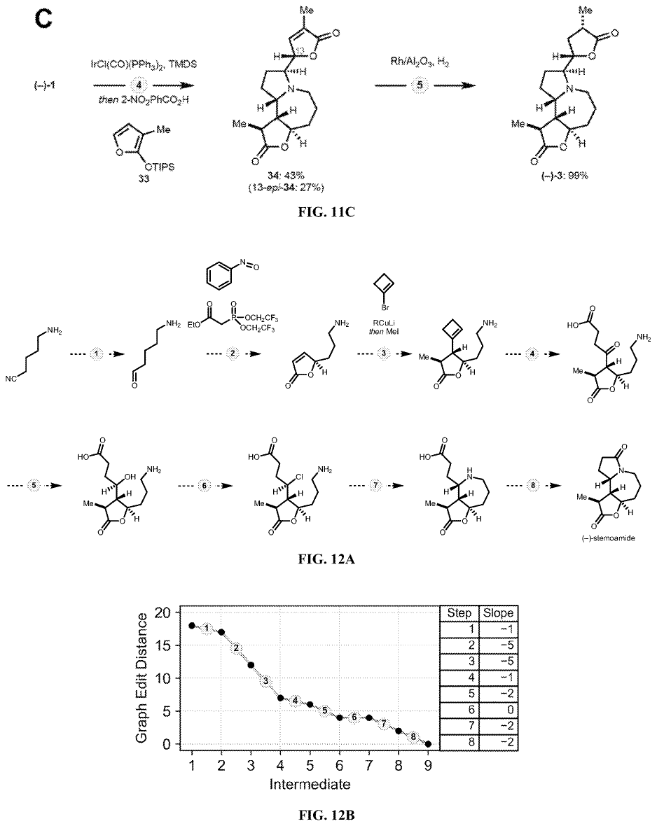

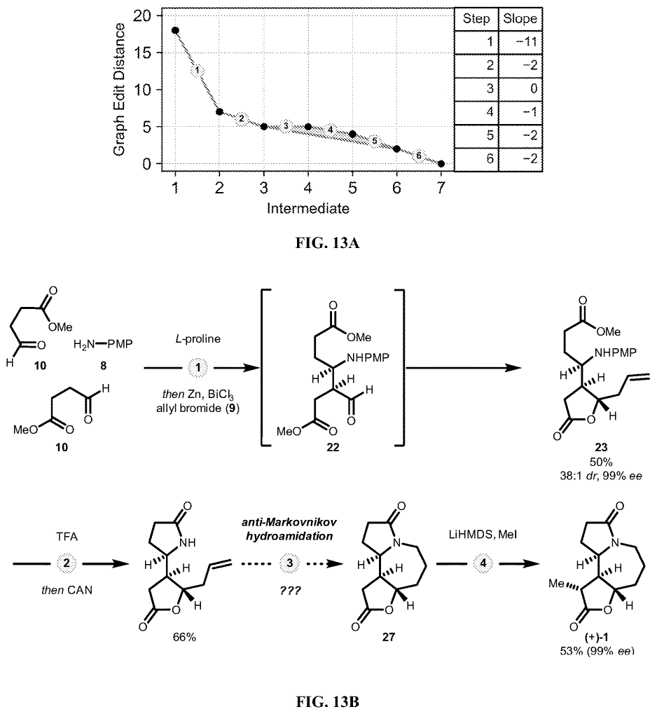

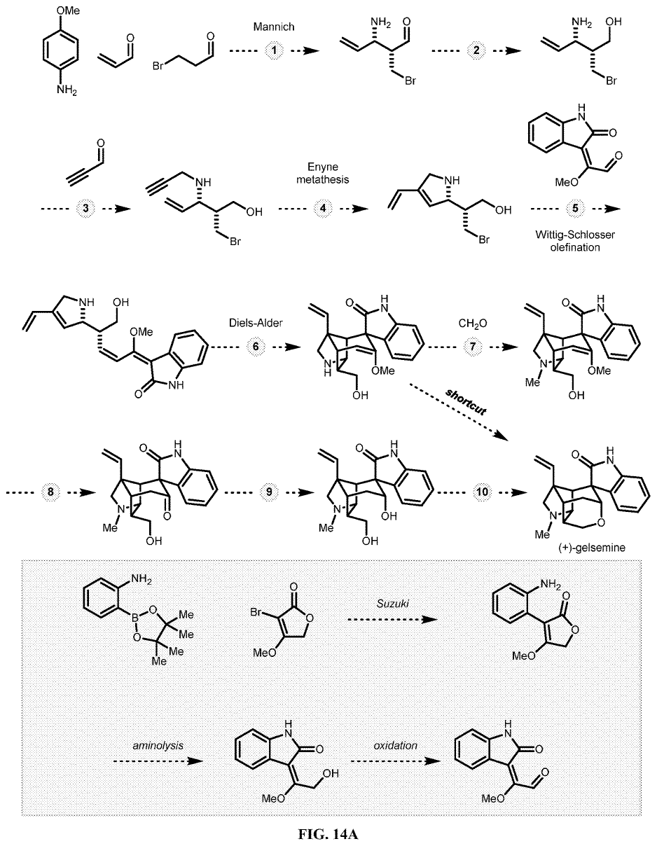

To best this result in terms of step-count, hundreds of additional calculated routes to 1 were generated, as well as related late-stage intermediates such as 27, to search for key steps that could be repurposed. The first synthesis (FIG. 10A) aimed to stay as true to the CASP-predicted route as possible, but some modifications were made as described. In a second route more liberties were taken, with focus on merging interesting key steps from multiple calculated routes. Graph edit distance analysis was used to direct attention to key steps of CASP-calculated routes and facilitate the combination of multiple key steps. One computed strategy of interest involved a peculiar cyclobutanone intermediate (an analog of 11, FIG. 11A), which was primed for Schmidt-rearrangement to access 1. Rather than utilize a proposed 4-step sequence to access an analog of key intermediate 11, it was recognized that an analogous diastereoselective Michael addition—allylation sequence (FIG. 2G) could be feasible to access 11 by adding cyclobutanone as a nucleophile into Michael acceptor 28 and quenching the intermediate enolate with methyl iodide. A similar Michael addition—alkylation sequence also emerged as a key step in a calculated route to 1 when the Mannich reaction was specifically excluded from the CASP search (FIG. 12). The Michael addition was performed via the Enders (S)-1-amino-2-methoxymethylpyrrolidine (SAMP) hydrazone of cyclobutanone (29), as the SAMP auxiliary method was frequently proposed in a variety of computed routes. Thus, the edited retrosynthetic strategy maximized high impact steps and minimized low impact steps to arrive at a concise proposal for the synthesis of 1.

Experimentally (FIG. 11), 28 was produced from commercially available 30 via Brown allylation in 89% ee and 58% yield. The optimized protocol for the conjugate addition comprised deprotonation of 29 with n-butyllithium, addition of 28 to a cold solution of the anion and trapping of the intermediate enolate with methyl iodide. Quenching of the reaction mixture with aqueous hydrochloric acid yielded ketone intermediate 11 in 88% yield as a 4:1 mixture of diastereomers. Subsequent iodosobenzoic acid (IBA) catalyzed anti-Markovnikov hydroazidation with trimethylsilyl azide gave 31, and Lewis-acid induced intramolecular Schmidt-Aubé rearrangement of presumed intermediate 32 led to (−)-1. The longest linear sequence was 3 steps giving the target in 22% overall yield, halving the prior step-count.

To demonstrate application to higher order stemona alkaloids, 1 was rapidly converted to target 3 (FIG. 11C) using Sato and Chida's conditions of treating with Vaska's complex in the presence of 1,1,3,3-tetramethyldisiloxane (TMDS) then exposing the reaction mixture to furan 33 and 2-nitrobenzoic acid in situ. Following chromatographic separation, the pure desired diastereomer 34 was obtained in 43% yield, as well as pure 13-epi-34 in 27% yield. Diastereoselective hydrogenation with rhodium on alumina delivered (−)-3 in 99% yield as a single stereoisomer.

Step impact can be readily observed in the 6-step synthesis of 1 where the organocatalyzed Mannich-allylation step dramatically increases the graph similarity of 8, 10, and allyl bromide to 1 installing 45% of the bonds required to produce 1 in the first step (FIG. 3B). This high impact step is then followed by a series of low impact steps, such as protecting group manipulations and functional group interconversions, which are easily recognized by their shallow slope in FIG. 10B. Meanwhile, the 3-step route is much more efficient with the sequence of steps contributing 17%, 55% and 28%, respectively, to the graph similarity of intermediates to 1 (FIG. 11B). The key steps used were selected from an analysis of more than a thousand calculated retrosynthetic routes.

In addition to highlighting key steps, the graph edit distance technique can be used to highlight shortcuts in the route, which may require the invention of new reactions. This is easily achieved by grouping neighboring transformations with a modest slope in the graph edit plot into a single shortcut step. For instance, steps 3, 4 and 5 in the Mannich route (FIG. 10) could in principle be grouped into an overall anti-Markovnikov hydroamidation (FIG. 13). It was possible to quench the TFA-promoted lactamization (step 2) with ceric ammonium nitrate (CAN) to produce an analog of 24 with the PMP group removed in 33% overall yield from 8. This analog could be converted to 27 in a single step following invention of an anti-Markovnikov hydroamidation reaction, ultimately leading to a 4-step synthesis of 1. This strategy can be generally applied to suggest specific new reactions, with their respective atom mappings, that provide a shortcut in any synthetic route.

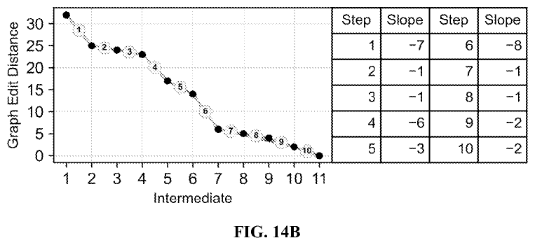

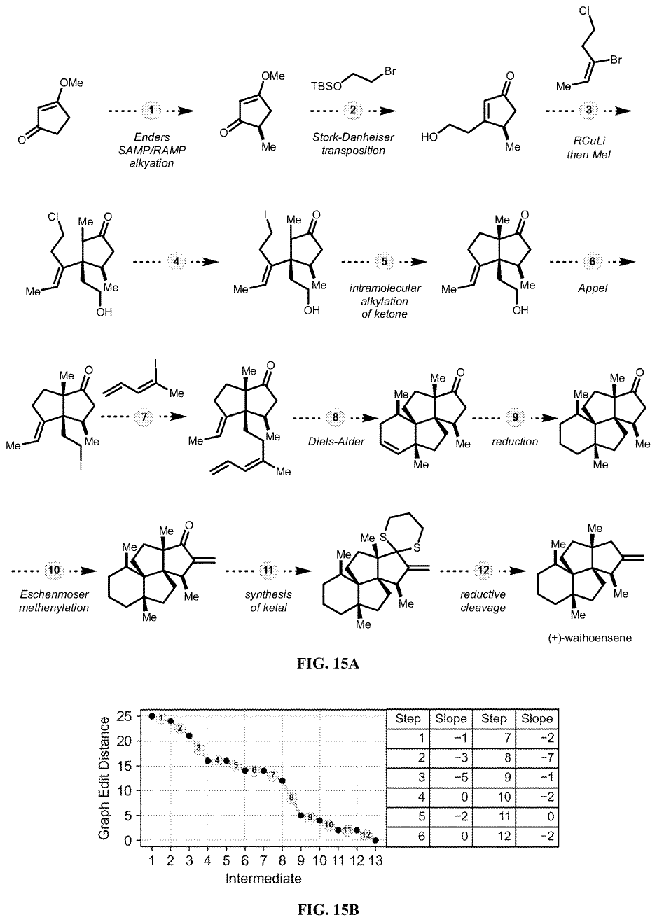

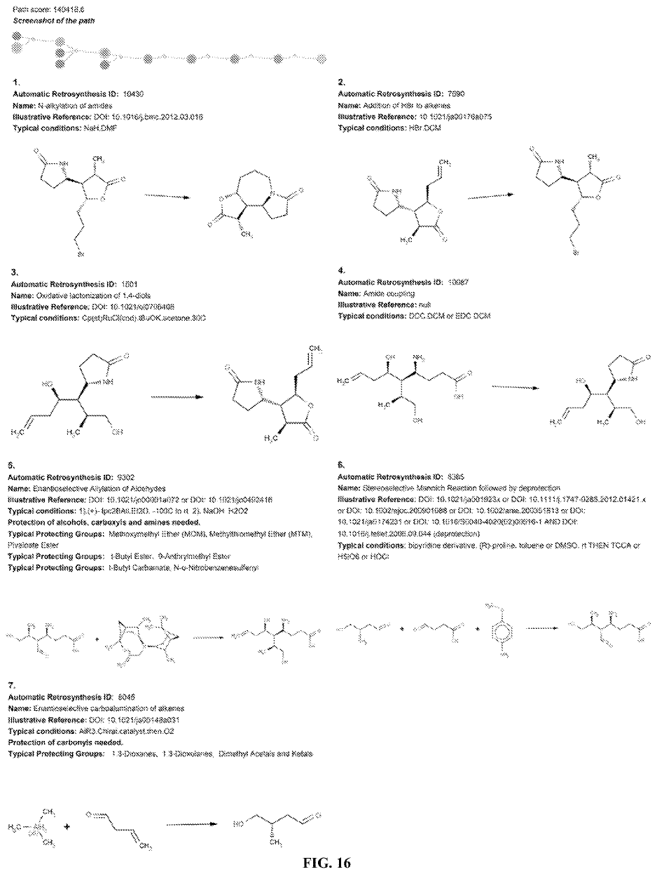

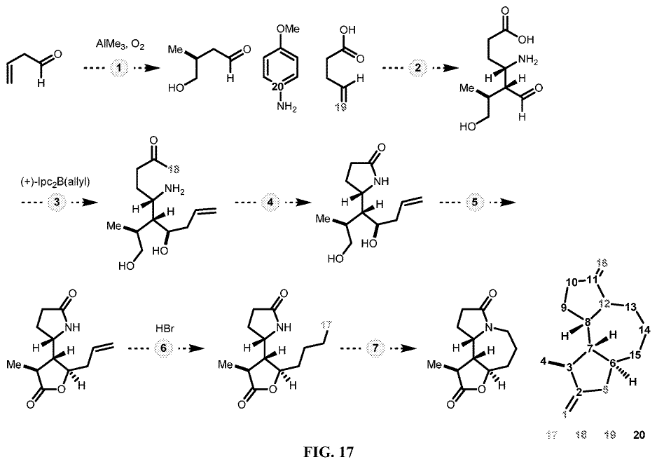

Application of graph edit distance analysis to other complex molecules was equally successful. For instance, a survey of 50 calculated routes to the alkaloid (+)-gelsemine revealed a 10-step proposed route (FIG. 14) that leveraged three highly simplifying transformations—an organocatalyzed Mannich reaction, an enyne metathesis, and a [4+2]cycloaddition—as key steps. Applying the undercutting strategy to the last 4-steps of the calculated route, invoking a reductive etherification, would lead to a proposal for a 7-step synthesis of gelsemine. Similarly, a 12-step retrosynthesis proposal for the diterpene (+)-waihoensene (FIG. 15) was derived.

Traditionally, convergent steps that join two or more building blocks that all contain target atoms, are valued in total synthesis. However, the matrix algorithm ignores convergent steps and non-convergent steps since only bond change is encoded. To showcase the value of convergent transformations, an additional scoring function, based on Maximum Common Substructure (MCS), was added to our distance metric. For every intermediate matrix, the largest fragment is first extracted, and the MCS between it and the target is computed. This is the largest collection of atoms that can be completely overlaid on the target, matching both atom identity and bond order. The MCS distance is then obtained by subtracting the number of heavy atoms in the MCS from that of the target.

The concept of MCS distance is illustrated in FIG. 51A through the synthesis of welwitindolinone. For each intermediate, the atoms and bonds constituting each intermediate's MCS with the target are highlighted in purple. On inspection, step 1 shows the greatest contribution to lowering MCS distance, as the size of the purple highlighted area increased the most. This can be formalized by plotting MCS distance against route (FIG. 51B), attributing the highest impact to the step with the steepest slope. As it is common for a convergent step to add more atoms to the intermediate, compared to the number of bonds made and broken in a single step, the MCS distance was halved before being added to the graph edit distance plot, making up the final distance metric. This strikes the satisfactory balance of highlighting both steps that are impactful through adding many target atoms, as well as steps that add many target bonds (FIG. 51C)

The methods disclosed herein show it is possible to unite multiple high impact steps from diverse CASP route proposals, as shown for (−)-1 in FIG. 11, to arrive at concise synthetic routes. Step count was the sole optimization metric in this Example, but important real-world metrics such as reagent cost, building block availability, or predicted yield could be easily incorporated as a weighted distance metric.

SYNTHIA™ Searches, Predicted Routes and their Graph Edit Distance Analysis

Several automatic retrosynthesis searches of (−)-stemoamide were performed in SYNTHIA™ (accessed Jan. 16, 2019). The parameters generating the route developed in FIG. 2F and FIG. 3, based on a key Mannich reaction, are as follows.

Automatic Retrosynthesis Results

| | |
|---|---|
| 1. | Reaction Scoring Function |

20000+40*PROTECT+1000000*(CONFLICT+NON_SELECTIVITY+FILTERS)

| | |
|---|---|
| 2. | Buyable $/g |

1000.0

| | |
|---|---|
| 3. | Known Popularity |

5

| | |
|---|---|
| 4. | Filters |
| 5. | Known g/mol |

1000.0

| | |
|---|---|
| 6. | Chemical Scoring Function |

SMALLER**3,SMALLER**1.5

| | |
|---|---|
| 7. | Buyable g/mol |

1000.0

| | |
|---|---|
| 8. | Retron smiles |

O═C1N2CCC[C@]3([H])[C@@]([C@H](C)C(O3)═O)([H])[C@]2([H])CC1

| | |
|---|---|
| 9. | Iterations |

0

| | |
|---|---|
| 10. | Bases |

Expert,

| | |
|---|---|
| 11. | Depth |

0

A proposal from the search invoking a key proline-catalyzed asymmetric cross-Mannich reaction of two aldehydes (retro-step 6) and an allylation of the Mannich product (retro-step 5). The SYNTHIA™ predicted route to (−)-1 with a key Mannich reaction (FIG. 16), as well as atom mapping in the route, are shown in FIG. 17, with the latter being used in constructing adjacency matrices and performing graph edit distance analysis in FIG. 7. Following General Procedures 1 and 2, the adjacency matrix for each starting material set, intermediate, and final product in the calculated route to (−)-1 (FIG. 3) was computed and shown in FIGS. 7A-7B, along with graph edit distance analysis of the calculated route.

The automatic retrosynthesis of norstemoamide (ent-27) was performed in SYNTHIA™ (accessed Jan. 17, 2019). The parameters generating the routes used in work, with a key Schmidt-Aubé rearrangement of a cyclobutanone intermediate, are as follows.

Automatic Retrosynthesis Results

| | |
|---|---|
| 1. | Reaction Scoring Function |

20000+40*PROTECT+1000000*(CONFLICT+NON_SELECTIVITY+FILTERS)

| | |
|---|---|
| 2. | Buyable $/g |

1000.0

| | |
|---|---|
| 3. | Known Popularity |

5

| | |
|---|---|
| 4. | Filters |

Strategies,

| | |
|---|---|
| 5. | Known g/mol |

1000.0

| | |
|---|---|
| 6. | Chemical Scoring Function |

SMALLER**3

| | |
|---|---|
| 7. | Buyable g/mol |

1000.0

| | |
|---|---|
| 8. | Retron smiles |

O═C1N2CCC[C@]3([H])[C@@](CC(O3)═O)([H])[C@]2([H])CC1

| | |
|---|---|
| 9. | Iterations |

0

| | |
|---|---|
| 10. | Bases |

Expert,

| | |
|---|---|
| 11. | Depth |

0

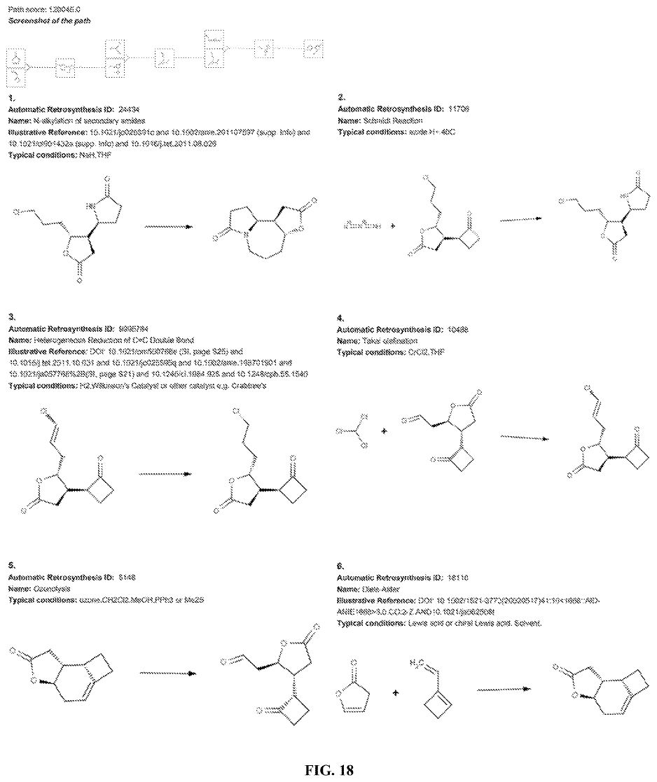

The top scoring route of the search is in FIG. 18 in which a Schmidt reaction of the cyclobutanone intermediate (retro-step 2) was proposed as a key step despite having a Diels-Alder reaction (retro-step 6) in the route deemed to be unlikely to succeed.

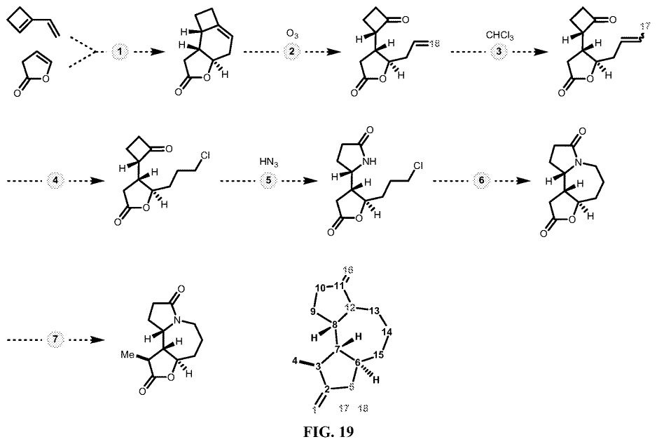

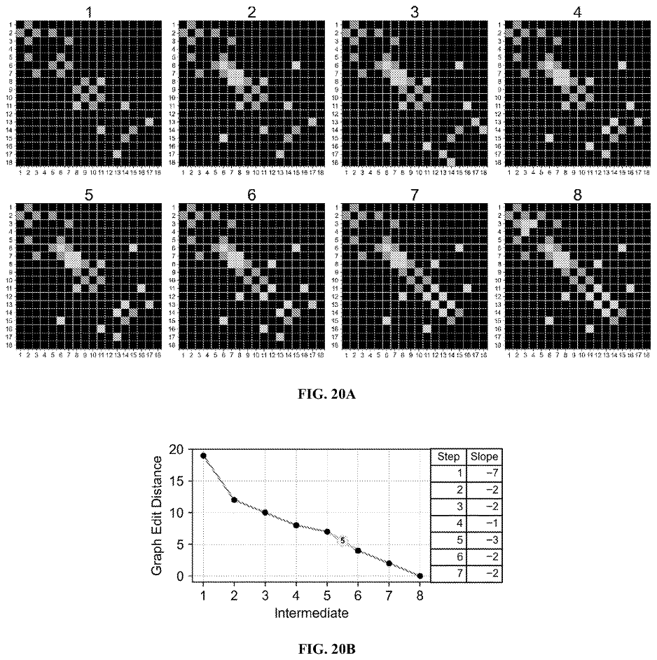

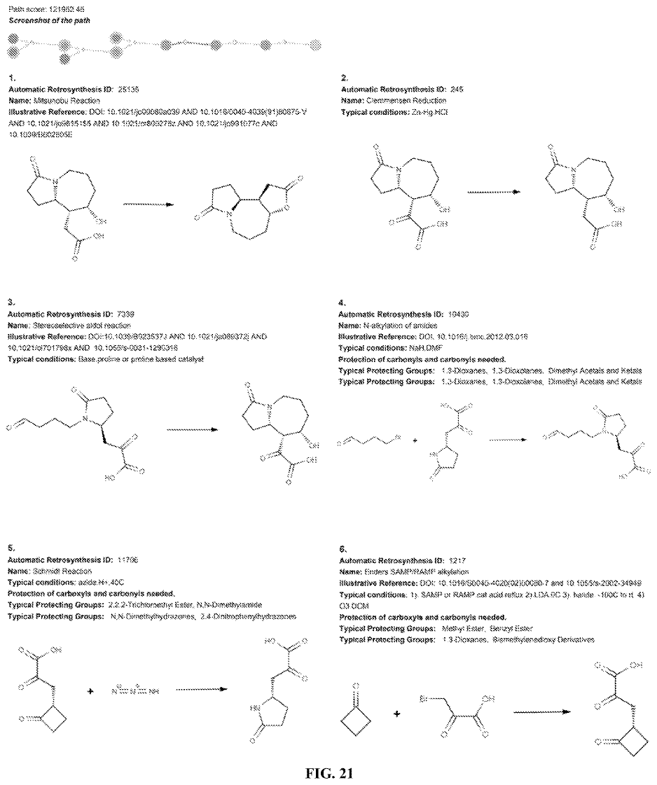

The SYNTHIA™ predicted route to (−)-1 with a key Schmidt reaction (FIG. 18), as well as atom mapping in the route, are shown in FIG. 19, with the latter being used in constructing adjacency matrices and performing graph edit distance analysis in FIG. 20. Following General Procedures 1 and 2, the adjacency matrix for each starting material set, intermediate, and final product in the calculated route to (−)-1 (FIG. 21) was computed, along with graph edit distance analysis of the calculated route.

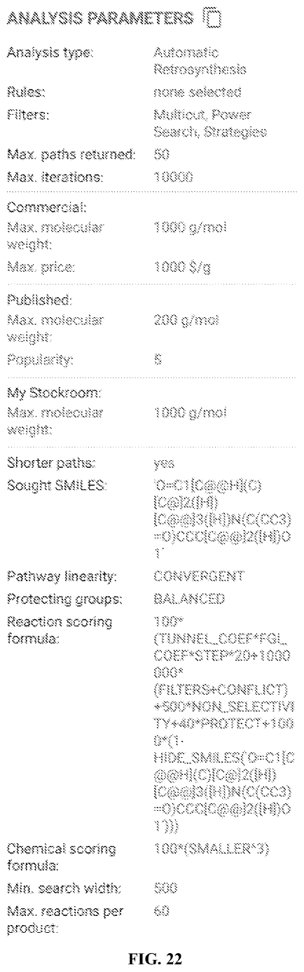

Another proposal generated by the search with an Enders SAMP hydrazone allylation (retro-step 6) and a Schmidt reaction (retro-step 5) is shown in FIG. 21. Several automatic retrosynthesis searches of (−)-stemonine (3) were performed in SYNTHIA™ (accessed Mar. 18, 2022). The parameters generating the route, based on a key Michael addition—alkylation, are shown in FIG. 22.

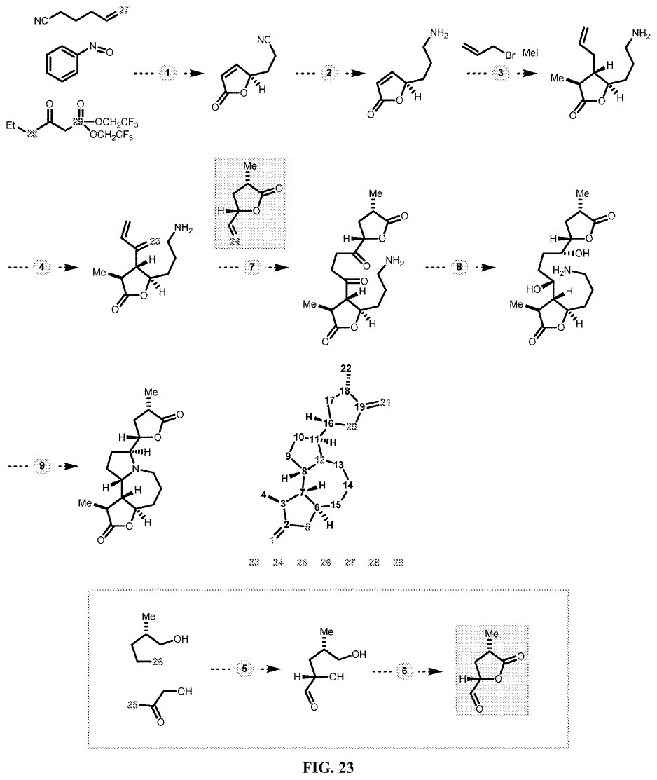

The SYNTHIA™ predicted route to (−)-3 with a key Michael addition—alkylation reaction, as well as atom mapping in the route is shown in FIG. 23, with the latter being used in constructing adjacency matrices and performing graph edit distance analysis in FIG. 9. Following General Procedures 1 and 2, the adjacency matrix for each starting material set, intermediate, and final product in the calculated route to (−)-3 (FIG. 23) was computed and shown in FIG. 9, along with graph edit distance analysis of the calculated route.

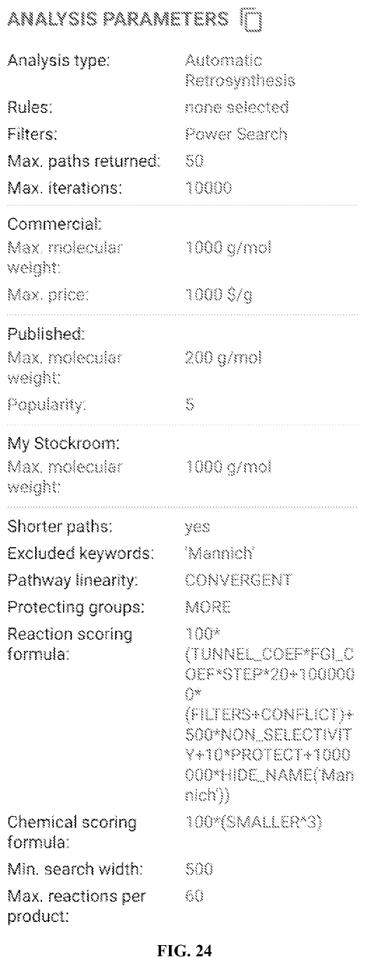

The automatic retrosynthesis of stemoamide (1) was performed in SYNTHIA™, with the Mannich reaction excluded (accessed Aug. 7, 2022). The parameters generating the routes are shown in FIG. 24. A Michael addition—methylation was identified as a key step.

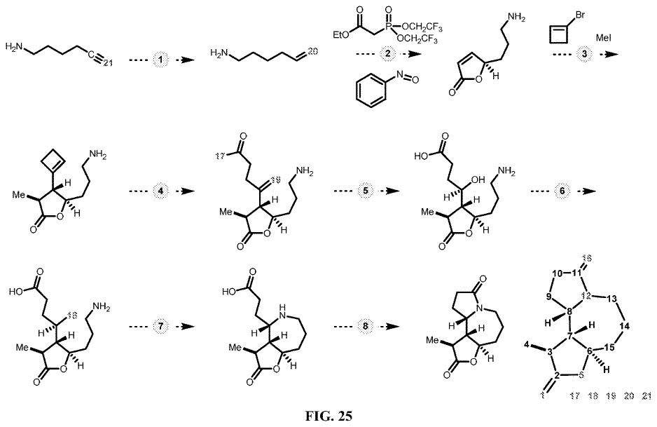

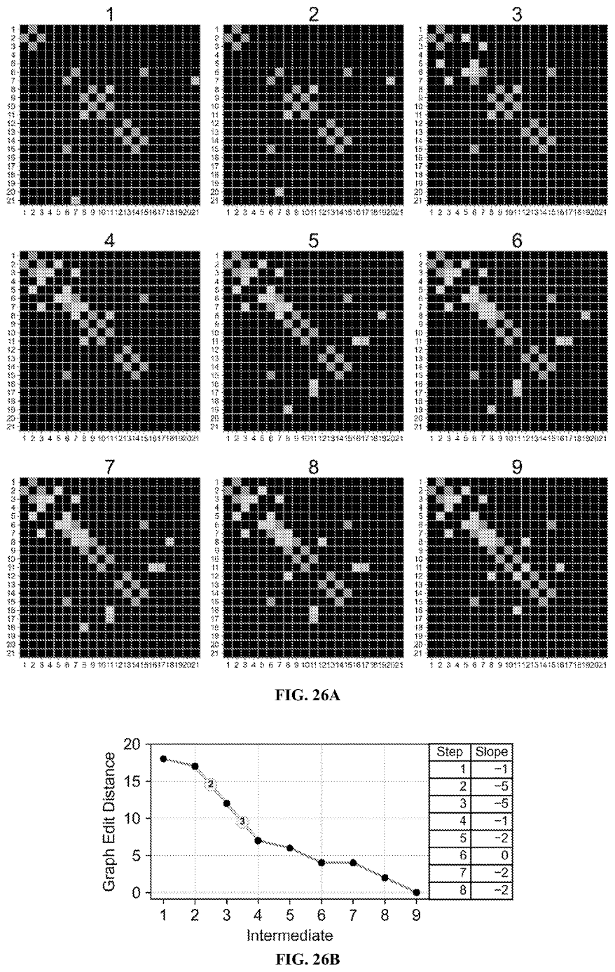

The SYNTHIA™ predicted route to (−)-1 with a key Michael addition—alkylation reaction when the Mannich reaction was excluded in the search, as well as atom mapping in the route, are shown in FIG. 25, with the latter being used in constructing adjacency matrices and performing graph edit distance analysis in FIG. 26. Following General Procedures 1 and 2, the adjacency matrix for each starting material set, intermediate, and final product in the calculated route to (−)-1 (FIG. 25) was computed and shown in FIG. 26, along with graph edit distance analysis of the calculated route.

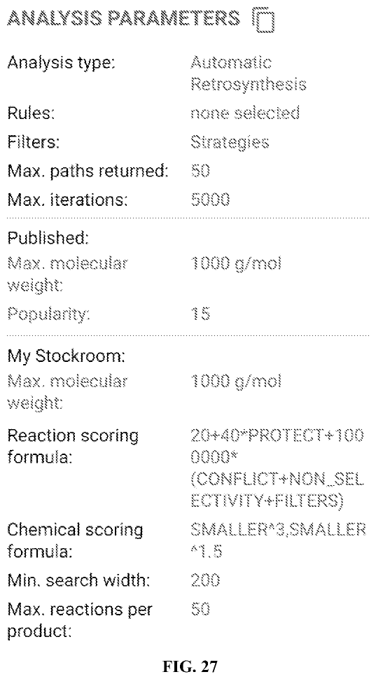

The automatic retrosynthesis of (+)-gelsemine was performed in SYNTHIA™ (accessed Jul. 21, 2020). The parameters generating the routes are as shown in FIG. 27. An organocatalyzed Mannich reaction, an enyne metathesis, and a [4+2]cycloaddition were identified as key steps.

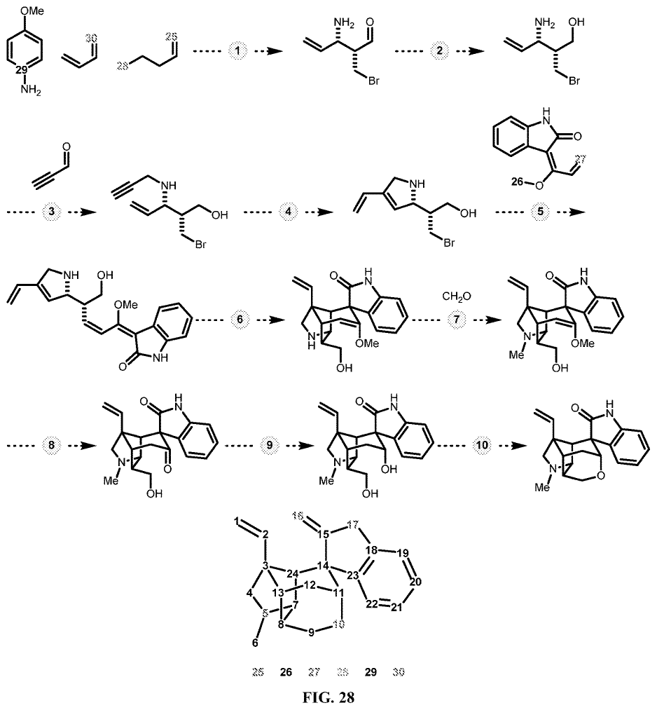

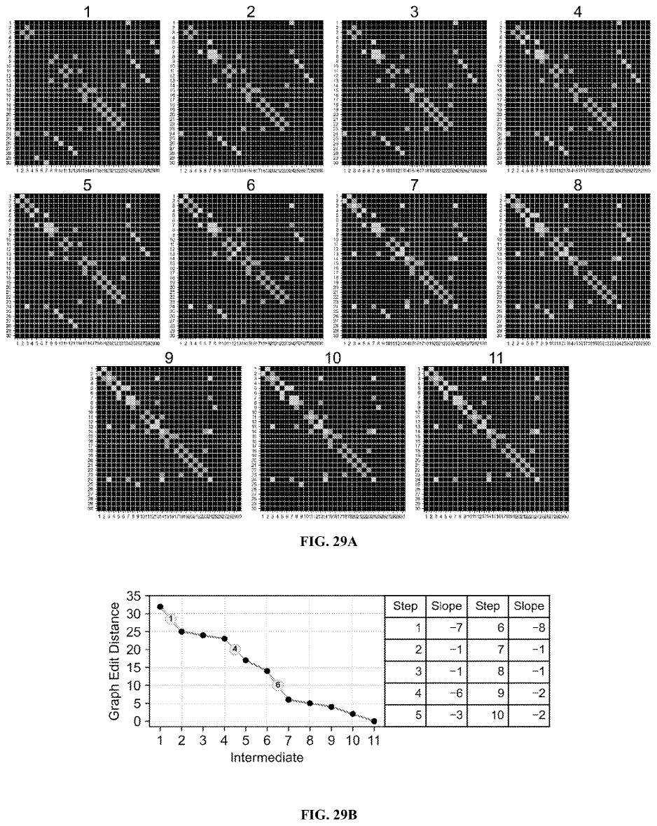

An organocatalyzed Mannich reaction, an enyne metathesis, and a [4+2]cycloaddition were identified as key steps. The SYNTHIA™ predicted route to (+)-gelsemine with an organocatalyzed Mannich reaction, an enyne metathesis, and a [4+2]cycloaddition as key steps, as well as atom mapping in the route, are shown in FIG. 28, with the latter being used in constructing adjacency matrices and performing graph edit distance analysis in FIG. 29. Following General Procedures 1 and 2, the adjacency matrix for each starting material set, intermediate, and final product in the calculated route to (+)-gelsemine (in FIG. 28) was computed and shown in FIG. 29, along with graph edit distance analysis of the calculated route.

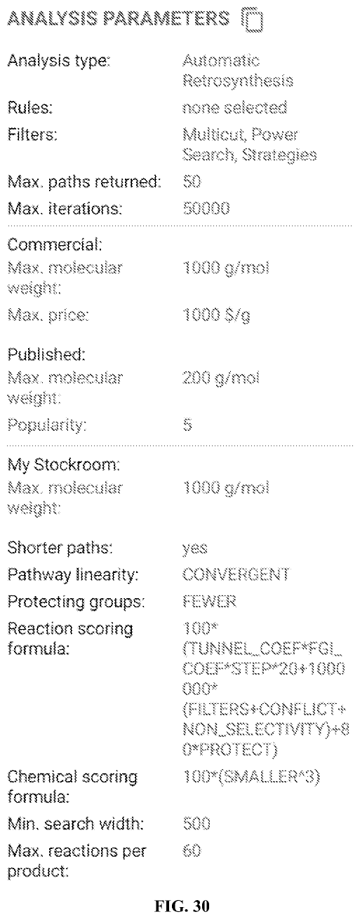

The automatic retrosynthesis of (+)-waihoensene was performed in SYNTHIA™ (accessed Oct. 5, 2021). The parameters generating the routes are as in FIG. 30. A Michael addition—alkylation and a [4+2]cycloaddition were identified as key steps.

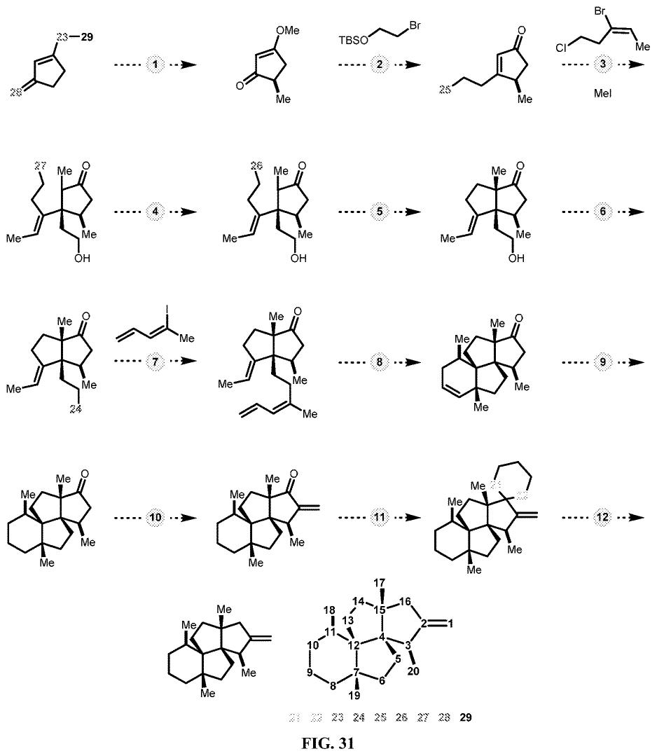

A Michael addition—alkylation and a [4+2]cycloaddition were identified as key steps. The SYNTHIA™ predicted route to (+)-waihoensene with a Michael addition—allylation and a [4+2]cycloaddition as key steps, as well as atom mapping in the route, are shown in FIG. 31, with the latter being used in constructing adjacency matrices and performing graph edit distance analysis in FIG. 32. Following General Procedures 1 and 2, the adjacency matrix for each starting material set, intermediate, and final product in the calculated route to (+)-waihoensene (FIG. 31) was computed and shown in FIG. 32, along with graph edit distance analysis of the calculated route.

### (R)-5-((2S,3S)-2-allyl-5-oxotetrahydrofuran-3-yl)-1-(4-methoxyphenyl)pyrrolidin-2-one (24)

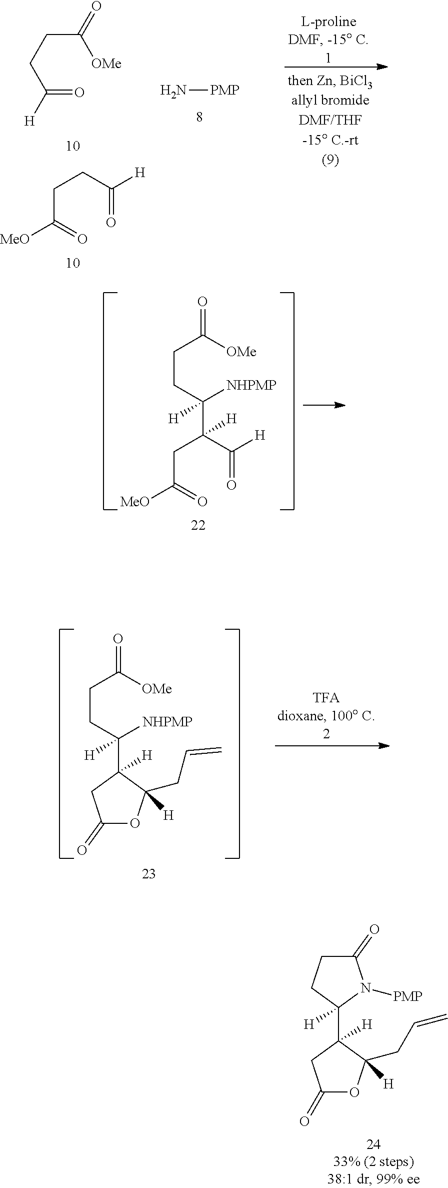

To a flame-dried 2-dram vial equipped with a stir bar were added 4-methoxyaniline (8) (246.0 mg, 2.000 mmol, 1 equiv) and L-proline (46.1 mg, 0.400 mmol, 20 mol %). After the vial was evacuated and backfilled with N₂ three times, anhydrous N,N-dimethylformamide (2.0 mL) was added via syringe. Then, the reaction mixture was cooled to −15° C. in a cold room. Methyl 4-oxobutanoate (10) (929.0 mg, 8.000 mmol, 4 equiv) was added to the reaction mixture at −15° C. and the mixture was stirred for 24 h at the same temperature.

To a flame-dried round bottom flask with a stir bar was added bismuth(III) chloride (5050.0 mg, 16.000 mmol, 8 equiv). Anhydrous THF (15.0 mL) and zinc (1570.0 mg, 24.000 mmol, 12 equiv) were subsequently added at room temperature. The mixture was stirred for 1 h at room temperature before the dropwise addition of allyl bromide (9) (1.4 mL, 16.000 mmol, 8 equiv).

The Mannich reaction mixture was transferred via syringe to the pre-stirred mixture of bismuth chloride, zinc, allyl bromide and THF at −15° C. Anhydrous THF (3.0 mL) was added to rinse the Mannich reaction mixture and transferred to the round bottom flask. The resulting reaction mixture was warmed to room temperature and stirred for 5 h. The reaction mixture was diluted with EtOAc (20.0 mL), filtered through Celite, and washed with EtOAc (40.0 mL). The filtrate was concentrated in vacuo, and the crude residue was redissolved in dioxane (20.0 mL) followed by the addition of TFA (0.8 mL, 10.000 mmol, 5 equiv) at room temperature. The mixture was heated at 100° C. for 10 minutes before being cooled to room temperature. The reaction mixture was diluted with EtOAc (20.0 mL), neutralized with saturated aqueous NaHCO₃ (30.0 mL), and the layers were separated. The aqueous layer was extracted with EtOAc (2×20.0 mL). The combined organic layers were washed with brine (30.0 mL), dried over anhydrous Na₂SO₄, filtered, and concentrated in vacuo. The crude residue was purified by flash chromatography (silica gel, eluent: 100% EtOAc) to afford 24 (207.0 mg, 33% yield, 38:1 dr, 99% ee) as a white solid.

¹H NMR (400 MHz, CDCl₃) δ 7.17 (d, J=8.9 Hz, 2H), 6.94 (d, J=9.0 Hz, 2H), 5.53 (ddt, J=17.2, 10.2, 7.1 Hz, 1H), 5.01 (d, J=9.9 Hz, 1H), 4.92 (dq, J=17.0, 1.5 Hz, 1H), 4.41-4.27 (comp, 2H), 3.81 (s, 3H), 2.70-2.50 (comp, 4H), 2.35-2.11 (comp, 3H), 2.07-1.96 (m, 1H), 1.82 (dddd, J=13.1, 9.7, 8.4, 6.9 Hz, 1H). ¹³C NMR (100 MHz, CDCl₃) δ 175.62, 174.68, 158.34, 131.30, 129.53, 126.19, 119.88, 114.83, 79.23, 60.96, 55.64, 39.76, 39.48, 30.82, 30.74, 20.47. HRMS (ESI): calculated C₁₈H₂₂NO₄⁺ [M+H]⁺: 316.1543, found: 316.1543. SFC: Daicel Chiralpak AS-H, column temperature=40° C., CO₂/iPrOH=70/30, Flow rate=3.5 mL/min, UV=238 nm, t_{R}=3.26 min (major) and t_{R}=6.00 min.

$${[ α ]}_{D}^{25.4} = + 3 7.4 ( c 1. , {CHCl}_{3} , 99 \% ee ) .$$

Racemic 24 was obtained following the above procedure by using DL-proline instead of L-proline. The spectral values matched the data above. (±)-24 was used for SFC analysis.

### (R)-5-((2S,3S)-2-(3-bromopropyl)-5-oxotetrahydrofuran-3-yl)pyrrolidin-2-one (26)

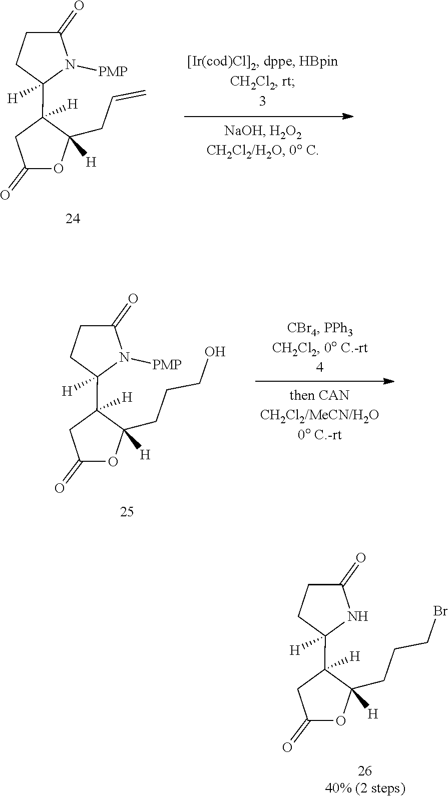

To a 25-mL round bottom flask equipped with a stir bar were added 24 (190.0 mg, 0.602 mmol, 1 equiv), [Ir(cod)Cl]2 (8.1 mg, 0.012 mmol, 2 mol %) and 1,2-bis(diphenylphosphaneyl)ethane (dppe) (9.6 mg, 0.024 mmol, 4 mol %) in a glovebox. Then, the flask was capped and moved out of the glovebox. Anhydrous CH₂Cl₂ (3.0 mL) was added via syringe, followed by the dropwise addition of 4,4,5,5-tetramethyl-1,3,2-dioxaborolane (122 μL, 0.846 mmol, 1.4 equiv). The mixture was stirred for 2 h at room temperature before the addition of THF (3.0 mL). The above mixture was cooled to 0° C. and a solution of NaOH (2 M, 452 μL, 0.904 mmol, 1.5 equiv) in water (3.0 mL) was added, followed by the addition of H₂O₂ (30%, 185 μL, 1.807 mmol, 3 equiv). The mixture was stirred at 0° C. for 30 minutes before being diluted with CH₂Cl₂ (10.0 mL). The layers were separated and the aqueous layer was extracted with CH₂Cl₂ (2×10.0 mL). The combined organic layers were washed with brine (10.0 mL), dried over anhydrous Na₂SO₄, filtered and concentrated in vacuo to afford crude 25, which was used directly for the next step without further purification.

To a solution of crude 25 (0.602 mmol, 1 equiv) in CH₂Cl₂ (10.0 mL) at 0° C. was added tetrabromomethane (240.0 mg, 0.723 mmol, 1.2 equiv) and triphenylphosphine (190.0 mg, 0.723 mmol, 1.2 equiv). The mixture was warmed to room temperature and stirred for 2 h before the addition of MeCN (3.0 mL). The mixture was then cooled to 0° C., a solution of ceric ammonium nitrate (CAN) (991.0 mg, 1.807 mmol, 3 equiv) in water (3.0 mL) added, and allowed to warm again to room temperature where it was stirred for 1 h. The layers were separated, and the aqueous layer extracted with CH₂Cl₂ (2×10.0 mL). The combined organic layers were washed with brine (5.0 mL), dried over anhydrous Na₂SO₄, filtered and concentrated in vacuo. The crude residue was purified by flash chromatography (silica gel, eluent: 100% EtOAc to 5% MeOH in EtOAc) to afford 26 (69.2 mg, 40% yield) as a white solid.

¹H NMR (700 MHz, CDCl₃) δ 7.52 (br s, 1H), 4.30 (ddd, J=9.5, 6.2, 3.4 Hz, 1H), 3.81 (q, J=6.7 Hz, 1H), 3.48 (dddd, J 16.5, 12.4, 10.2, 5.6 Hz, 2H), 2.69 (dd, J 17.9, 9.1 Hz, 1H), 2.46 (dd, J=17.8, 7.5 Hz, 1H), 2.41-2.27 (comp, 4H), 2.15-2.06 (m, 1H), 2.04-1.97 (m, 1H), 1.97-1.90 (m, 1H), 1.80-1.71 (m, 2H). ¹³C NMR (175 MHz, CDCl₃) δ 179.13, 175.12, 81.03, 55.28, 45.92, 33.79, 33.19, 30.54, 30.25, 28.53, 25.33. HRMS (ESI): calculated C₁₁H₁₇⁷⁹BrNO₃⁺ [M+H]⁺: 290.0386, found: ⁷⁹Br 290.0385, ⁸¹Br 292.0367.

$${[ α ]}_{D}^{25.6} = - 1 9.66 ( c 0.37 , {CHCl}_{3} , 99 \% ee ) .$$

### Norstemoamide (27)

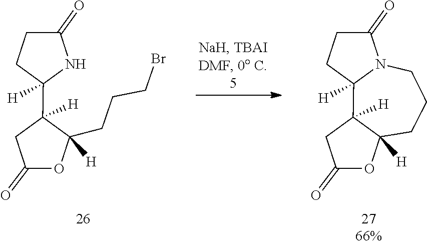

To a mixture of 26 (21.0 mg, 0.072 mmol, 1 equiv) and tetrabutylammonium iodide (TBAI) (2.7 mg, 0.007 mmol, 0.1 equiv) in anhydrous DMF (1.4 mL) at 0° C. was added sodium hydride (60%, 4.4 mg, 0.108 mmol, 1.5 equiv). The mixture was stirred for 2 h at 0° C. before being quenched with 2 M HCl (0.2 mL). The mixture was diluted with water (10.0 mL) and extracted with EtOAc (5×5.0 mL). The combined layers were dried over anhydrous Na₂SO₄, filtered and concentrated in vacuo. The crude residue was purified by flash chromatography (silica gel, eluent: 5-10% MeOH/EtOAc) to afford 27 (10.0 mg, 66% yield) as a colorless oil.

¹H NMR (500 MHz, CDCl₃) δ 4.28 (td, J 10.2, 3.0 Hz, 1H), 4.14 (dt, J=13.1, 3.1 Hz, 1H), 3.99 (dt, J 10.5, 6.4 Hz, 1H), 2.84 (dtd, J 12.6, 9.4, 6.8 Hz, 1H), 2.70-2.60 (comp, 2H), 2.51 (dd, J=17.3, 12.7 Hz, 1H), 2.45-2.36 (comp, 3H), 2.06 (dtt, J=11.6, 5.8, 2.7 Hz, 1H), 1.89-1.82 (m, 1H), 1.77-1.63 (m, 1H), 1.62-1.49 (comp, 2H). ¹³C NMR (125 MHz, CDCl₃) δ 174.81, 174.21, 79.92, 56.20, 45.05, 40.34, 34.78, 31.14, 30.70, 25.62, 22.81. HRMS (ESI): calculated C₁₁H₁₆NO₃⁺ [M+H]⁺: 210.1125, found: 210.1127.

$${[ α ]}_{D}^{25.5} = + 1 3.04 ( c 0.51 , {CHCl}_{3} , 99 \% ee ) .$$

### (+)-Stemoamide ((+)-1)

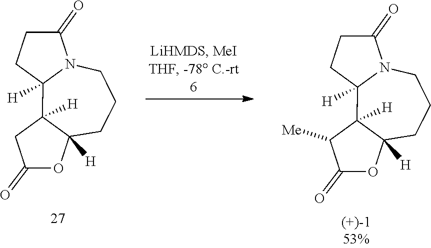

To a solution of 27 (10.0 mg, 0.048 mmol, 1 equiv) in THF (0.3 mL) was added a solution of LiHMDS (9.6 mg, 0.057 mmol, 1.2 equiv) in THF (0.2 mL) dropwise at −78° C. (dry ice/acetone bath). The mixture was stirred for 2 h at the same temperature before the dropwise addition of Mel (4.5 μL, 0.072 mmol, 1.5 equiv). After 15 min at −78° C., the mixture was warmed to room temperature and stirred for 2 h. Then the mixture was quenched with 2 M HCl (0.2 mL) and extracted with EtOAc (5×3.0 mL). The combined layers were dried over anhydrous Na₂SO₄, filtered and concentrated in vacuo. The crude residue was purified by flash chromatography (silica gel, eluent: 5% MeOH in EtOAc) to afford (+)-1 (5.7 mg, 53% yield) as a white solid.

¹H NMR (700 MHz, CDCl₃) δ 4.20 (td, J 10.3, 3.0 Hz, 1H), 4.15 (dt, J=14.8, 3.1 Hz, 1H), 3.99 (dt, J=10.7, 6.4 Hz, 1H), 2.68-2.63 (m, 1H), 2.59 (dq, J=13.8, 7.0 Hz, 1H), 2.45-2.35 (comp, 4H), 2.04 (dq, J=15.7, 5.2 Hz, 1H), 1.90-1.82 (m, 1H), 1.71 (p, J=10.9 Hz, 1H), 1.58-1.46 (comp, 2H), 1.30 (d, J=6.9 Hz, 3H). ¹³C NMR (125 MHz, CDCl₃) δ 177.51, 174.15, 77.78, 55.96, 52.86, 40.38, 37.47, 34.97, 30.77, 25.78, 22.73, 14.26. HRMS (ESI): calculated C₁₂H₁₈NO₃⁺ [M+H]⁺: 224.1281, found: 224.1280. SFC: Daicel Chiralcel OJ-H, column temperature=40° C., CO₂/iPrOH=80/20, Flow rate=3.5 mL/min, UV=210 nm, t_{R}=2.35 min and t_{R}=2.72 min (major).

$${[ α ]}_{D}^{25.8} = + 15 0.47 ( c 0.39 , MeOH , 99 \% ee ) .$$

The absolute configuration was assigned by comparison of the optical rotation with our data of (−)-1

$${[ α ]}_{D}^{26.0} = - 13 5.41 ( c 0.48 , MeOH , 92 \% ee ) ,$$

see below for details] and confirmed by comparison of our SFC data of (+)-1 and (−)-1 obtained from separate routes.

### Methyl (R)-4-((2S,3S)-2-allyl-5-oxotetrahydrofuran-3-yl)-4-((4-methoxyphenyl)amino)butanoate (23)

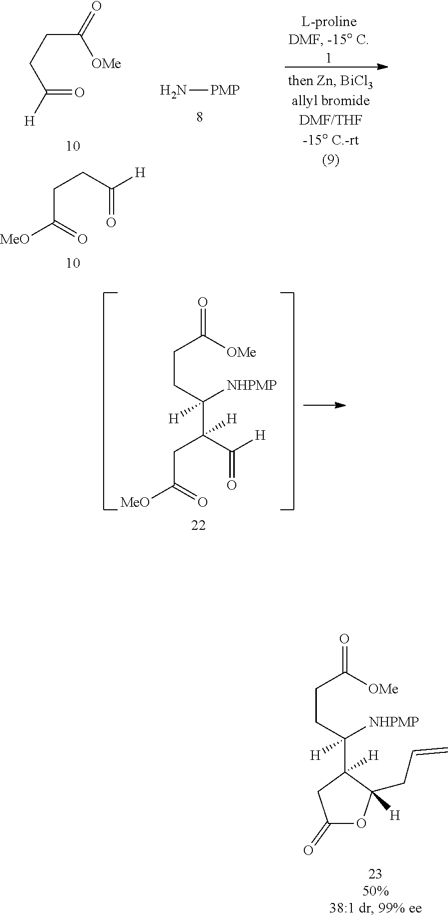

To a flame-dried 2-dram vial equipped with a stir bar were added 4-methoxyaniline (8) (615.8 mg, 5.000 mmol, 1 equiv) and L-proline (115.1 mg, 1.000 mmol, 20 mol %). After the vial was evacuated and backfilled with N₂ three times, anhydrous N,N-dimethylformamide (5.0 mL) was added via syringe. Then the reaction mixture was cooled to −15° C. in a cold room. Methyl 4-oxobutanoate (10) (2322.0 mg, 20.000 mmol, 4 equiv) was added to the reaction mixture at −15° C. and the mixture was stirred for 24 h at the same temperature.

To a flame-dried round bottom flask with a stir bar was added bismuth(III) chloride (12610.0 mg, 40.000 mmol, 8 equiv). Anhydrous THF (40.0 mL) and zinc (3923.0 mg, 60.000 mmol, 12 equiv) were subsequently added at room temperature. The mixture was stirred for 1 h at room temperature before the dropwise addition of allyl bromide (9) (3.5 mL, 40.000 mmol, 8 equiv).

The Mannich reaction mixture was transferred via syringe to the pre-stirred mixture of bismuth chloride, zinc, allyl bromide and THF at −15° C. Anhydrous THF (5.0 mL) was added to rinse the Mannich reaction mixture and transferred to the round bottom flask. The resulting reaction mixture was warmed to room temperature and stirred for 5 h. The reaction mixture was diluted with EtOAc (50.0 mL), filtered through Celite, and washed with EtOAc (100.0 mL). The filtrate was washed with saturated aqueous NH₄Cl (60.0 mL) and the layers were separated. The aqueous layer was extracted with EtOAc (2×50.0 mL). The combined organic layers were washed with brine (80.0 mL), dried over anhydrous Na₂SO₄, filtered and concentrated in vacuo. The crude residue was purified by flash chromatography (silica gel, eluent: 30% EtOAc/hexanes) to afford 23 (867.0 mg, 50% yield, 38:1 dr, 99% ee) (ee was determined by converting a small fraction of 23 to 24 in the presence of TFA in dioxane).

¹H NMR (400 MHz, CDCl₃) δ 6.74 (d, J=8.9 Hz, 2H), 6.50 (d, J=8.9 Hz, 2H), 5.75 (ddt, J=16.5, 10.8, 7.0 Hz, 1H), 5.19-5.09 (m, 2H), 4.44 (q, J=5.7 Hz, 1H), 3.73 (s, 3H), 3.61 (s, 3H), 3.50-3.40 (m, 1H), 3.29 (br s, 1H), 2.65-2.35 (comp, 7H), 1.84 (dtd, J=14.4, 7.3, 4.0 Hz, 1H), 1.75-1.63 (m, 1H). ¹³C NMR (100 MHz, CDCl₃) δ 176.13, 173.85, 152.44, 141.48, 132.13, 119.41, 115.21, 114.33, 81.27, 55.86, 54.17, 51.85, 44.36, 39.14, 30.86, 30.40, 29.04. HRMS (ESI): calculated C₁₉H₂₅NNaO₅⁺ [M+Na]⁺: 370.1625, found: 370.1626.

### (R)-5-((2S,3S)-2-allyl-5-oxotetrahydrofuran-3-yl)pyrrolidin-2-one (S1)

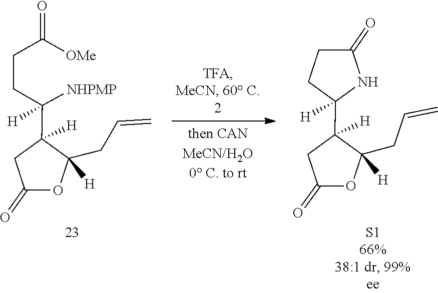

To a solution of compound 23 (418.0 mg, 1.203 mmol, 1 equiv) in acetonitrile (6.0 mL) was added TFA (463 μL, 6.016 mmol, 5 equiv) at room temperature. The mixture was heated at 60° C. for 3 h before it was cooled to 0° C., and a solution of ceric ammonium nitrate (CAN) (1979.0 mg, 3.610 mmol, 3 equiv) in water (6.0 mL) added dropwise. The mixture was allowed to warm to room temperature and stirred for 1 h. Then the reaction mixture was diluted with EtOAc (10.0 mL), and the layers were separated. The aqueous layer was extracted with EtOAc (2×10.0 mL). The combined organic layers were washed with brine (10.0 mL), dried over anhydrous Na₂SO₄, filtered and concentrated in vacuo. The crude residue was purified by flash chromatography (silica gel, eluent: 100% EtOAc to 5% MeOH in EtOAc) to afford S1 (167.0 mg, 66% yield) as a white solid.

¹H NMR (400 MHz, CDCl₃) δ 7.93 (br s, 1H), 5.75 (ddt, J 18.9, 9.8, 7.0 Hz, 1H), 5.21-5.11 (m, 2H), 4.35 (q, J=5.7 Hz, 1H), 3.75 (q, J=6.7 Hz, 1H), 2.64 (dd, J 17.8, 9.0 Hz, 1H), 2.50-2.18 (comp, 7H), 1.74-1.61 (m, 1H). ¹³C NMR (100 MHz, CDCl₃) δ 179.30, 175.63, 131.68, 119.78, 80.95, 55.46, 44.30, 38.96, 30.39, 30.20, 25.08. HRMS (ESI): calculated C₁₁H₁₅NNaO₃⁺ [M+Na]⁺: 232.0944, found: 232.0944.

### A SYNTHIA™ Proposed Synthetic Route to 7, Involving a Cyclobutanone Expansion of S2.

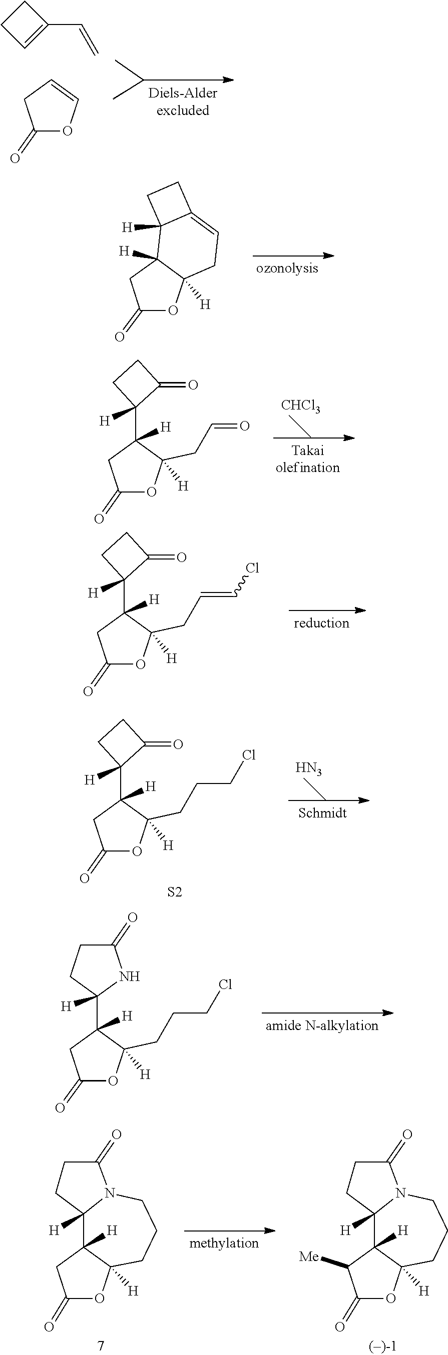

However, the proposed synthesis to make S2 involves a Diels-Alder reaction, which likely would not succeed as it would form the wrong diastereomer provided 2(3H)-furanone did not isomerize under the Diels-Alder reaction conditions. Thus, a different approach to access 11 (an analogue to S2) was proposed and experimentally realized.

### Experimental Attempt for the Catalytic Enantioselective Allylation to Make Compound 28

Selective studies were shown in Table 2 for the catalytic enantioselective allylation to prepare 28 from commercial reagents. Although enantiomeric excess is often excellent even with an impressively low catalyst and ligand loading (see highlighted entry in Table 2: 94% ee, 0.5 mol % [Pd] and 0.75 mol % L, respectively), the isolated yield of 28 is frustratingly low under all conditions, mainly due to the competing α-allylation side reaction (for example, see the highlighted result in red in Table 2, 66% yield of SP was isolated), The low yielding result drove us to find a new allylation method to prepare compound 28.

General procedure for experiments in Table 2: A mixture of [Pd] catalyst and (S,S)-DACH-phenyl Trost ligand (L) in the indicated solvent was stirred for 1 h at room temperature. The above [Pd]/L solution was transferred into a solution of nucleophile Nu (Nu=2-(trimethylsilyloxy)furan, 2-(tert-butyldimethylsilyloxy)furan, or 2-(triisopropylsilyloxy)furan) in the indicated solvent at the indicated temperature, followed by the addition of a solution of electrophile E (E=allyl acetate, allyl methyl carbonate or allyl tert-butyl carbonate) or slow addition of it via syringe pump. After completion, judged by TLC analysis, the crude mixture was purified by flash chromatography directly (silica gel, eluent: 25% EtOAc/hexane) to yield 28. Enantiomeric excess (ee) was determined by SFC analysis.

TABLE 2
Optimization of catalytic enantioselective allylation

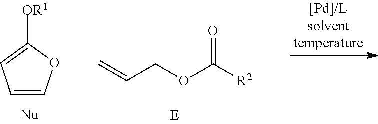

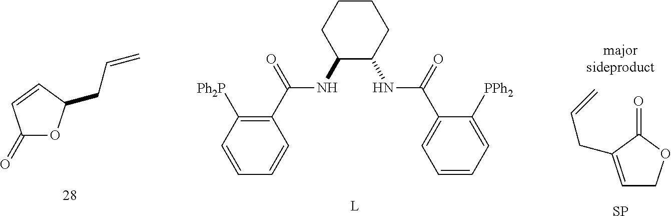

| Nu | E | | | L | | | Temp | Yield | % |
|---|---|---|---|---|---|---|---|---|---|
| R¹ | R² | Nu:E | [Pd] | mol % | Special setup | Solvent | (° C.) | (%) | ee |
| TMS | Me | 1:1 | Pd2dba3 (5 | 12 | none | PhMe(0.5 M) | 25 | <15 | 67 |
| | | | mol %) | | | | | | |
| TMS | OMe | 1:1 | Pd2dba3 (5 | 12 | none | PhMe (0.5 M) | 25 | <15 | 72 |
| | | | mol %) | | | | | | |
| TBS | Me | 1:2 | Pd2dba3 (5 | 12 | none | PhMe (0.5 M) | 25 | <15 | 35 |
| | | | mol %) | | | | | | |
| TBS | OMe | 1:2 | Pd2dba3 (5 | 12 | none | PhMe (0.5 M) | 25 | <15 | 52 |
| | | | mol %) | | | | | | |
| TIPS | Me | 1:2 | Pd2dba3 (5 | 12 | none | PhMe (0.5 M) | 25 | <15 | 20 |
| | | | mol %) | | | | | | |
| TIPS | OMe | 1:2 | Pd2dba3 (5 | 12 | none | PhMe (0.5 M) | 25 | <15 | 25 |
| | | | mol %) | | | | | | |
| TMS | OMe | 1:1.5 | Pd2dba3 (5 | 12 | slow addition of | CH₂Cl₂ (0.5 M) | 0-4 | 23 | 91 |
| | | | mol %) | | E over 1 h | | | | |
| TMS | OtBu | 1:1.5 | Pd2dba3 (5 | 12 | slow addition of | CH₂Cl₂ (0.5 M) | 0 | 21 | 95 |
| | | | mol %) | | E over 1 h | | | | |
| TMS | OtBu | 1:1.5 | Pd2dba3 (5 | 12 | slow addition of | CH₂Cl₂ (0.5 M) | 4 | 27 | 95 |
| | | | mol %) | | E over 1 h | | | | |
| TMS | OtBu | 1:1.5 | Pd2dba3 (5 | 12 | slow addition of | CH₂Cl₂ (0.5 M) | −15 | 25 | 97 |
| | | | mol %) | | E over 1 h | | | | |
| TMS | OtBu | 1:1 | Pd2dba3 (5 | 12 | slow addition of | CH₂Cl₂ (0.5 M) | 4 | 21 | ND |
| | | | mol %) | | E over 1 h | | | | |
| TMS | OtBu | 1:1 | Pd2dba3 (5 | 12 | slow addition of | CH₂Cl₂ (0.5 M) | 4 | 22 | ND |
| | | | mol %) | | E over 1 h | | | | |
| TMS | OtBu | 1:1 | Pd2dba3 | 7.5 | slow addition of | CH₂Cl₂ (0.5 M) | 0 | 22 | 95 |
| | | | CHCl₃ | | E over 1 h | | | (SP | |
| | | | (2.5 mol %) | | | | | 66%) | |
| TMS | OtBu | 1:1 | Pd2dba3 | 7.5 | slow addition | CH₂Cl₂ (0.5 M) | 0 | 12 | 92 |
| | | | CHCl₃ (2.5 | | of the mixture | | | | |
| | | | mol %) | | of Nu + E (1:1) | | | | |
| | | | | | over 1 h | | | | |
| TMS | OtBu | 1:1 | Pd2dba3 | 7.5 | slow addition of | CH₂Cl₂ (0.5 M) | 0 | 16 | 94 |
| | | | CHCl₃ (2.5 | | E over 4 h | | | | |
| | | | mol %) | | | | | | |
| TMS | OtBu | 1:1 | Pd2dba3 CH | 7.5 | slow addition of | CH₂Cl₂ (0.5 M) | 0 | 21 | 95 |
| | | | Cl₃ (2.5 | | E over 0.25 h | | | | |
| | | | mol %) | | | | | | |
| TMS | OtBu | 1:1 | Pd2dba3 CH | 0.75 | slow addition of | CH₂Cl₂ (1 M) | 0 | 20 | 94 |
| | | | Cl₃ (0.25 | | E over 1 h | | | | |
| | | | mol %) | | | | | | |
| TMS | OtBu | 1:1 | [Pd(allyl)Cl]₂ | 0.75 | slow addition of | CH₂Cl₂ (1 M) | 0 | 11 | 85 |
| | | | (0.25 mol %) | | E over 1 h | | | | |

### (S)—N-(2-(methoxymethyl)pyrrolidin-1-yl)cyclobutanimine (29)

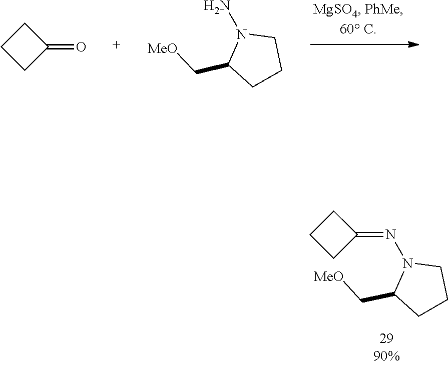

Following a modified literature procedure (D. Hazelard, A. Fadel, S Tetrahedron: Asymmetry 16, 2067-2070 (2005)), to a mixture of anhydrous magnesium sulfate (0.481 g, 4.000 mmol, 1 equiv) in anhydrous toluene (4.0 mL) were added (S)-(−)-1-amino-2-(methoxymethyl)pyrrolidine (SAMP) (537 μL, 4.000 mmol, 1 equiv, 97% ee), cyclobutanone (389 μL, 5.200 mmol, 1.3 equiv), and trifluoroacetic acid (31 μL, 0.400 mmol, 0.1 equiv) at room temperature. The mixture was heated at 60° C. for 3 h before it was cooled to room temperature. The reaction was quenched with water (2.0 mL), neutralized with saturated aqueous NaHCO₃ (5.0 mL), and extracted with EtOAc (5×20.0 mL). Note: the desired product has a relatively good solubility in water, so it is beneficial to extract the aqueous layer multiple times with EtOAc. The combined organic layers were dried over anhydrous Na₂SO₄, filtered and concentrated in vacuo. The crude residue was purified by flash chromatography (silica gel, eluent: 40% EtOAc/hexane) to afford 29 (654.0 mg, 90% yield) as a yellow oil.

¹H NMR (500 MHz, CDCl₃) δ 3.53 (dd, J=9.3, 3.9 Hz, 1H), 3.41-3.31 (comp, 4H), 3.27 (qd, J=7.3, 3.8 Hz, 1H), 3.05-2.82 (comp, 4H), 2.61 (q, J=8.6 Hz, 1H), 1.99-1.89 (comp, 3H), 1.88-1.78 (m, 2H), 1.72-1.62 (m, 2H). ¹³C NMR (125 MHz, CDCl₃) δ 155.26, 75.42, 65.97, 59.38, 53.73, 36.31, 35.37, 26.21, 22.60, 14.25. HRMS (ESI): calculated C₁₀H₁₉N₂O⁺ [M+H]⁺: 183.1492, found: 183.1492.

### (R)-5-allylfuran-2(5H)-one (28)

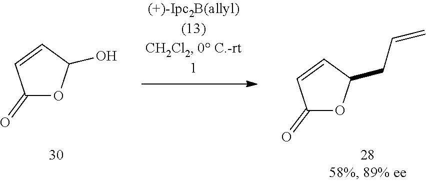

To a solution of 5-hydroxyfuran-2(5H)-one 30 (93.1 mg, 0.930 mmol, 1 equiv) in anhydrous CH₂Cl₂ (14.0 mL) was added a solution of (+)-Ipc₂B(allyl)borane 13 (1 M in pentane, 1.4 mL, 1.395 mmol, 1.5 equiv) in CH₂Cl₂ (2.2 mL) dropwise at 0° C. After addition, the mixture was warmed to room temperature and stirred for 48 h before concentration in vacuo. The crude residue was purified by flash chromatography (silica gel, eluent: 25% EtOAc/hexane) to afford 28 (66.5 mg, 58% yield, 89% ee) as a colorless liquid.

¹H NMR (500 MHz, CDCl₃) δ 7.46 (dd, J=5.7, 1.5 Hz, 1H), 6.14 (dd, J=5.7, 2.0 Hz, 1H), 5.76 (ddt, J=17.2, 10.4, 7.0 Hz, 1H), 5.24-5.15 (m, 2H), 5.08 (tt, J=6.5, 1.7 Hz, 1H), 2.59-2.44 (m, 2H). ¹³C NMR (125 MHz, CDCl₃) δ 172.96, 155.78, 131.08, 122.17, 119.76, 82.45, 37.40. SFC: Daicel Chiralpak AS-H, column temperature=40° C., CO₂/iPrOH=95/5, Flow rate=3.5 mL/min, UV=210 nm, t_{R}=1.80 min and t_{R}=1.96 min (major).

$${[ α ]}_{D}^{25.8} = - 117.88 ( c 1.09 , MeOH , 89 \% ee ) .$$

The characterization data matched spectral values from literature.

The absolute configuration was assigned by comparison of the optical rotation with published data of (S)-5-allylfuran-2(5H)-one

$$( {[ α ]}_{D}^{0} = + 1 \; 05 ( c 1.08 , MeOH ) ) .$$

Racemic 28 was obtained following a modified literature procedure (B. M. Trost, C.-I. Hung, M. J. Scharf, Angewandte Chemie International Edition 57, 11408-11412 (2018)):

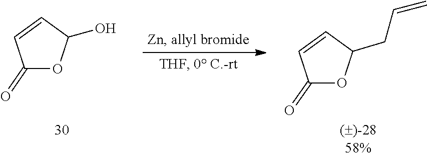

To a solution of 30 (1001.0 mg, 10.000 mmol, 1 equiv) in anhydrous THF (10.0 mL) were added allyl bromide (1038 μL, 12.000 mmol, 1.2 equiv) and zinc powder (1961.0 mg, 30.000 mmol, 3 equiv) at 0° C. The reaction mixture was subsequently warmed to room temperature and stirred for 22 h before being quenched with saturated aqueous NH₄Cl (30.0 mL). The mixture was then filtered through Celite to remove the zinc residue and the layers were separated. The aqueous layer was extracted with EtOAc (2×30.0 mL). The combined organic layers were washed with brine (30.0 mL), dried over anhydrous Na₂SO₄, filtered and concentrated in vacuo. The crude residue was purified by flash chromatography (silica gel, eluent: 25% EtOAc/hexane) to afford (±)-28 (717.0 mg, 58% yield) as a colorless liquid. The characterization data matched spectral values of 28 obtained above.

### (3S,4S,5R)-5-allyl-3-methyl-4-((S)-2-oxocyclobutyl)dihydrofuran-2(3H)-one (11, major) and (3S,4S,5R)-5-allyl-3-methyl-4-((R)-2-oxocyclobutyl)dihydrofuran-2(3H)-one (9a-epi-11, minor) (4:1 dr)

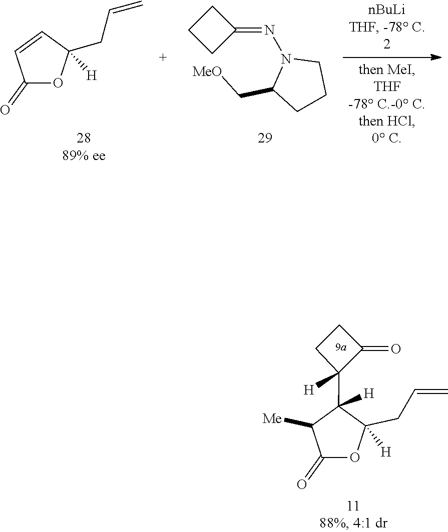

Following a modified literature procedure (D. Enders, K. Papadopoulos, Tetrahedron Letters 24, 4967-4970 (1983) and D. Enders, et al., Tetrahedron Letters 27, 3491-3494 (1986)), to a solution of 29 (43.7 mg, 0.240 mmol, 1.2 equiv) in anhydrous degassed THF (degassing: anhydrous THF was sparged with N₂ for 15 minutes prior to use) (1.0 mL) was added n-BuLi (2.63 M in hexanes, 91 μL, 0.240 mmol, 1.2 equiv) dropwise via micro-syringe at −78° C. (dry ice/acetone bath). The mixture was stirred at the same temperature for 1 h. To the above mixture was added a solution of 28 (24.8 mg, 0.200 mmol, 1 equiv) in anhydrous degassed THF (0.2 mL) dropwise at −78° C. THF (0.2 mL) was added to rinse the flask containing 28 and transferred to the reaction flask at −78° C. The mixture was stirred at the same temperature for 2 h before the dropwise addition of Mel (19 μL, 0.300 mmol, 1.5 equiv). The resulting mixture was stirred at −78° C. for 30 minutes, then warmed to 0° C. (ice bath) and stirred for another 30 minutes. EtOAc (1.5 mL) and 2 M HCl (0.5 mL) were added to the reaction mixture sequentially at 0° C. After 30 minutes at 0° C., the layers were separated, and the aqueous layer extracted with EtOAc (2×2.0 mL). The combined organic layers were washed with brine (3.0 mL), dried over anhydrous Na₂SO₄, filtered and concentrated in vacuo. The crude residue was purified by flash chromatography (silica gel, eluent: 25% EtOAc/hexane) to afford 11 (30.0 mg, 72% yield, 4.5:1 dr) as a yellow oil. To the above aqueous HCl layer was added EtOAc (1.5 mL), and the mixture was stirred at room temperature for 3 h before the layers were separated, and the aqueous layer extracted with EtOAc (2×2.0 mL). The combined organic layers were washed with brine (3.0 mL), dried over anhydrous Na₂SO₄, filtered and concentrated in vacuo. The crude residue was purified by flash chromatography (silica gel, eluent: 25% EtOAc/hexane) to afford 11 (6.7 mg, 16% yield, 3.9:1 dr) as a yellow oil. In total, 36.7 mg of two inseparable diastereomers were obtained with a dr of 4:1 and a combined yield of 88%.

### Characterization Data of the Mixture:

¹H NMR (500 MHz, CDCl₃) δ 5.85-5.73 (m, 1H, major+minor), 5.21-5.11 (comp, 2H, major+minor), 4.30-4.19 (m, 1H, major+minor), 3.53-3.42 (m, 1H, major+minor), 3.17-3.05 (m, 1H, major+minor), 2.97-2.85 (m, 1H, major+minor), 2.72-2.47 (comp, 2H, major+minor), 2.47-2.30 (m, 1H, major+minor), 2.30-2.19 (m, 1H, major+minor), 2.19-2.08 (m, 1H, major+minor), 1.93-1.79 (m, 1H, major+minor), 1.33 (d, J=7.1 Hz, 3H, Me of major), 1.27 (d, J=7.2 Hz, 3H, Me of minor). ¹³C NMR (125 MHz, CDCl₃) δ 207.96 (major), 207.80 (minor), 178.25 (major), 178.08 (minor), 132.48 (minor), 132.27 (major), 119.32 (major), 119.22 (minor), 80.84 (minor), 80.47 (major), 61.18 (minor), 60.91 (major), 47.13 (major), 47.01 (minor), 45.08 (minor), 44.91 (major), 39.78 (minor), 39.39 (major), 38.57 (major), 38.12 (minor), 15.48 (major), 15.46 (minor), 15.08 (major), 14.49 (minor). HRMS (ESI): calculated C₁₂H₁₅O₃⁻ [M−H]⁻: 207.1027, found: 207.1026.

A racemic 1.1:1 diastereomeric mixture of (+)-11 and (±)-9a-epi-11 was obtained using cyclobutanone N,N-dimethylhydrazone S3 instead of 29.

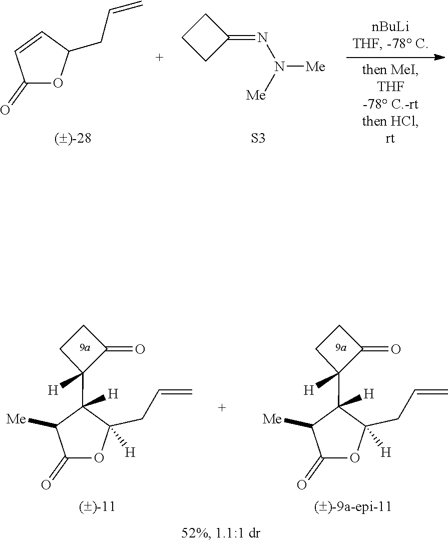

Following a modified literature procedure (D. Enders, K. Papadopoulos, Tetrahedron Letters 24, 4967-4970 (1983) and D. Enders, et al., Tetrahedron Letters 27, 3491-3494 (1986)), to a solution of S3 (15) (442.0 mg, 3.940 mmol, 1 equiv) in anhydrous degassed THF (15.0 mL) was added n-BuLi (2.28 M in hexanes, 1728 μL, 3.940 mmol, 1 equiv) dropwise via syringe at −78° C. (dry ice/acetone bath). The mixture was stirred at the same temperature for 30 minutes. Then the mixture was warmed to 0° C. and stirred for 1 h before it was re-cooled down to −78° C., and a solution of (+)-28 (489.0 mg, 3.940 mmol, 1 equiv) in anhydrous degassed THF (5.0 mL) added dropwise. THF (0.2 mL) was added to rinse the flask containing (+)-28 and transferred to the reaction flask at −78° C. The mixture was stirred at the same temperature for 2 h before the dropwise addition of Mel (294 μL, 4.730 mmol, 1.2 equiv). The resulting mixture was warmed to room temperature and stirred for 1 h. Then, EtOAc (30.0 mL) and 2 M HCl (10.0 mL) were added to the reaction mixture sequentially. After the mixture was stirred for 2 h at room temperature, the layers were separated and the aqueous layer was extracted with EtOAc (2×30.0 mL). The combined organic layers were washed with brine (30.0 mL), dried over anhydrous Na₂SO₄, filtered and concentrated in vacuo. The crude residue was purified by flash chromatography (silica gel, eluent: 25% EtOAc/hexane) to afford (±)-11 and (±)-9a-epi-11 as a 1.1:1 diastereomeric mixture (427.0 mg, 52% yield, 1.1:1 dr) as a yellow oil. The characterization data matched spectral values of 11 obtained above.

### (−)-Stemoamide and (−)-9a-epi-stemoamide ((−)-1 and (−)-9a-epi-1)

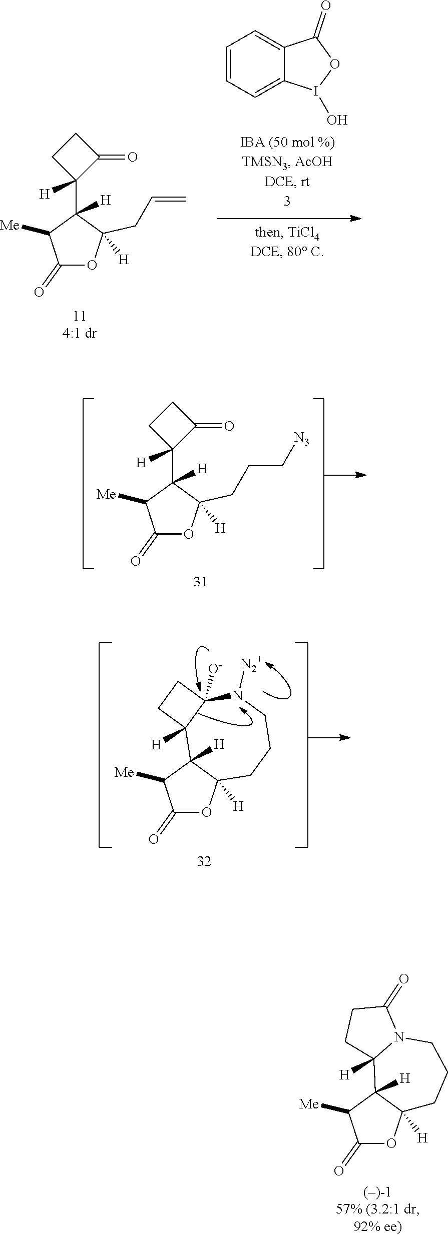

Following a modified literature procedure (H. Li, et al., Journal of the American Chemical Society 141, 9415-9421 (2019), X. Li, et al., Science China Chemistry 62, 1537-1541 (2019), and K. J. Frankowski, et al., Angewandte Chemie International Edition 54, 10555-10558 (2015)), to a 4:1 mixture of 11 (28.0 mg, 0.134 mmol, 1 equiv) in anhydrous DCE (0.1 mL) were added iodosobenzoic acid (IBA) (18.1 mg, 0.067 mmol, 50 mol %), azidotrimethylsilane (56 μL, 0.403 mmol, 3 equiv) and acetic acid (23 μL, 0.403 mmol, 3 equiv) sequentially under N₂ at room temperature. The mixture was stirred at room temperature for 20 h. To this mixture was added anhydrous DCE (2.1 mL), followed by the dropwise addition TiCl₄ (1 M in DCE, 0.4 mL, 0.403 mmol, 3 equiv) at room temperature. The resulting mixture was heated at 80° C. for 4 h before it was cooled to room temperature and quenched with saturated aqueous NaHCO₃ (3.0 mL). The mixture was filtered, and the layers were separated. The aqueous layer was extracted with EtOAc (3×3.0 mL). The combined organic layers were washed with brine (3.0 mL), dried over anhydrous Na₂SO₄, filtered and concentrated in vacuo. The crude residue was purified by flash chromatography (silica gel, eluent: 100% EtOAc to 5% MeOH in EtOAc) to afford (−)-1 and (−)-9a-epi-1 (17.2 mg, 57% yield, 3.2:1 dr) as a white solid. For analytical samples, two diastereomers were separated by PrepLC on Teledyne ISCO CombiFlash® EZ Prep (RediSep Prep C18, 100 A, 5 μm, 150 mm×20 mm (partno. 692203810), eluent: gradient from 100% H₂O to 20% MeOH/H₂O). Solvent methanol was removed in vacuo by a rotary evaporator and water was removed in vacuo through lyophilization.

### Characterization Data of (−)-Stemoamide ((−)-1):

¹H NMR (700 MHz, CDCl₃) δ 4.20 (td, J 10.3, 3.0 Hz, 1H), 4.15 (dt, J 14.8, 3.1 Hz, 1H), 3.99 (dt, J=10.7, 6.4 Hz, 1H), 2.68-2.63 (m, 1H), 2.59 (dq, J=13.8, 7.0 Hz, 1H), 2.45-2.35 (comp, 4H), 2.04 (dq, J=15.7, 5.2 Hz, 1H), 1.90-1.82 (m, 1H), 1.71 (p, J=10.9 Hz, 1H), 1.58-1.46 (comp, 2H), 1.30 (d, J=6.9 Hz, 3H). ¹³C NMR (175 MHz, CDCl₃) δ 177.51, 174.14, 77.77, 55.95, 52.85, 40.37, 37.46, 34.96, 30.76, 25.77, 22.72, 14.26. HRMS (ESI): calculated C₁₂H₁₈NO₃⁺ [M+H]⁺: 224.1281, found: 224.1280. SFC: Daicel Chiralcel OJ-H, column temperature=40° C., CO₂/iPrOH=80/20, Flow rate=3.5 mL/min, UV=210 nm, t_{R}=2.35 min (major) and t_{R}=2.74 min.

$${[ α ]}_{D}^{26.0} = - 13 5.41 ( c 0.48 , MeOH , 92 \% ee ) .$$

Note, the observed ee value increase through the sequence, from 28 (89% ee) to (−)-1 (92% ee), was likely due to kinetic resolution via a reactivity difference of the two enantiomers of 28 in the conjugate addition with lithiated enantiopure 29. This hypothesis is also supported by the observed ee decrease of (−)-9a-epi-1 (75% ee) from 28 (89% ee). [literature values:

$${[ α ]}_{D}^{20} = - 213.1 ( c 0.5 , MeOH , 98 \% ee ) ( 19 ) ;$$

$${[ α ]}_{D}^{20} = - 138 ( c 0.2 , MeOH ) ( 20 ) ;$$

$${[ α ]}_{D}^{20} = - 179.7 ( c 0.09 , MeOH ) ( 21 ) ;$$

$${[ α ]}_{D}^{25} = - 183.5 ( c 1.36 , MeOH ) ( 22 ) ;$$

$${[ α ]}_{D}^{20} = - 141 ( c 0.3 , MeOH ) ( 23 ) ;$$

$${[ α ]}_{D}^{27} = - 151.58 ( c 0.46 , MeOH ) ( 24 ) ;$$

$${[ α ]}_{D}^{30} = - 219.3 ( c 0.5 , MeOH ) ( 25 ) ;$$

$${[ α ]}_{D}^{25} = - 187 ( c 0.5 , MeOH ) ( 26 ) ;$$

$${[ α ]}_{D}^{25} = - 191.6 ( c 0.5 , MeOH ) ( 27 ) ;$$

$${[ α ]}_{D}^{21} = - 180.7 ( c 0.89 , MeOH ) ( 28 ) ;$$

$${[ α ]}_{D}^{26} = - 141 ( c 0.19 , MeOH ) ( 29 ) ;$$

$$- 181 ( c 0.89 , MeOH ) ( 29 ) ;$$

$${[ α ]}_{D}^{21.6} = - 28.1 ( c 0.125 , MeOH ) ( 30 ) and$$

$${[ α ]}_{D}^{21} = - 162. ( c 0.5 , {CHCl}_{3} ) ( 31 ) . ]$$

Characterization Data of (−)-9a-Epi-Stemoamide ((−)-9a-Epi-1):

¹H NMR (700 MHz, CDCl₃) δ 4.31 (td, J=10.5, 5.2 Hz, 1H), 3.89 (ddd, J=14.7, 6.7, 3.0 Hz, 1H), 3.58 (td, J 9.0, 6.5 Hz, 1H), 3.14 (ddd, J=13.9, 9.9, 2.7 Hz, 1H), 2.54-2.44 (comp, 3H), 2.44-2.37 (m, 1H), 2.24 (dddd, J=12.0, 9.0, 6.5, 2.8 Hz, 1H), 1.94 (q, J=10.2 Hz, 1H), 1.90-1.78 (comp, 3H), 1.76-1.68 (m, 1H), 1.39 (d, J=7.0 Hz, 3H). ¹³C NMR (175 MHz, CDCl₃) δ 177.63, 174.30, 81.20, 62.18, 55.37, 40.34, 39.95, 30.88, 30.45, 24.97, 22.43, 15.53. HRMS (ESI): calculated C₁₂H₁₈NO₃⁺ [M+H]⁺: 224.1281, found: 224.1280. SFC: Daicel Chiralcel OJ-H, column temperature=40° C., CO₂/iPrOH=80/20, Flow rate=3.5 mL/min, UV=210 nm, t_{R}=3.04 min (major) and t_{R}=3.85 min.

$${[ α ]}_{D}^{26.0} = - 1 2.2 ( c , 0.15 , MeOH , 75 \% ee ) .$$

The characterization data of (−)-9a-epi-stemoamide matched spectral values from literature. The relative configuration of (−)-9a-epi-stemoamide was assigned by comparison of the ¹H and ¹³C NMR data with published data of 9a-epi-stemoamide. The assignment was also supported by analysis of gCOSY and NOESY spectra of (−)-stemoamide and (−)-9a-epi-stemoamide (see below and spectra for details).

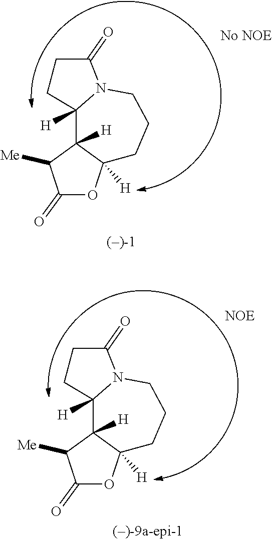

A 1.1:1 diastereomeric mixture of (+)-1 and (±)-9a-epi-1 was obtained following the above procedure using the mixture of (+)-11 and (±)-9a-epi-11 (1.1:1 dr). The spectral values matched the data above and the mixture (1.1:1 dr) of (±)-1 and (±)-9a-epi-1 was used for SFC analysis.

### Gram Scale Synthesis of (−)-stemoamide ((−)-1)

### (R)-5-allylfuran-2(5H)-one (28)

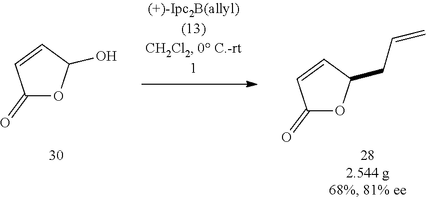

To a solution of 5-hydroxyfuran-2(5H)-one 30 (3.002 g, 30.000 mmol, 1 equiv) in anhydrous CH₂Cl₂ (480.0 mL) was added a solution of (+)-Ipc₂B(allyl)borane 13 (1 M in pentane, 45.0 mL, 45.000 mmol, 1.5 equiv) in CH₂Cl₂ (75.0 mL) dropwise at 0° C. After addition, the mixture was warmed to room temperature and stirred for 20 h before concentration in vacuo. The crude residue was purified by flash chromatography (silica gel, eluent: 20% EtOAc/hexane) to afford 28 (2.544 g, 68% yield, 81% ee) as a colorless liquid.

The characterization data matched spectral values of 28 obtained above.

### (3S,4S,5R)-5-allyl-3-methyl-4-((S)-2-oxocyclobutyl)dihydrofuran-2(3H)-one (major) and (3S,4S,5R)-5-allyl-3-methyl-4-((R)-2-oxocyclobutyl)dihydrofuran-2(3H)-one (minor) (22, 2:1 dr)

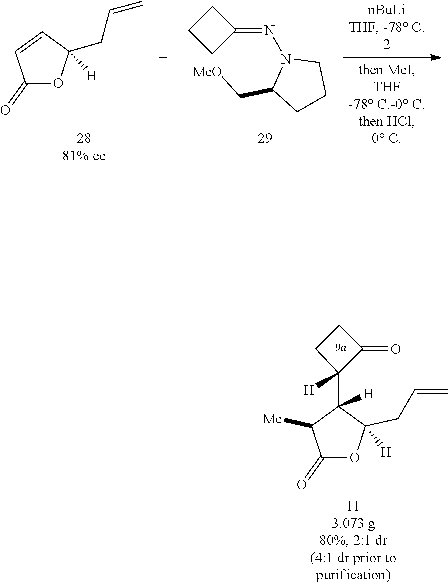

Following a modified literature procedure (D. Enders, K. Papadopoulos, Tetrahedron Letters 24, 4967-4970 (1983) and D. Enders, et al., Tetrahedron Letters 27, 3491-3494 (1986)), to a solution of 29 (4.046 g, 22.200 mmol, 1.2 equiv) in anhydrous degassed THF (92.5 mL) was added n-BuLi (2.70 M in hexanes, 8.2 mL, 22.200 mmol, 1.2 equiv) dropwise via syringe at −78° C. (dry ice/acetone bath). The mixture was stirred at the same temperature for 1 h. To the above mixture was added a solution of 28 (2.297 g, 18.500 mmol, 1 equiv) in anhydrous degassed THF (15.0 mL) dropwise (over about 30 min) at −78° C. THF (10.0 mL) was added to rinse the flask containing 28 and transferred to the reaction flask at −78° C. The mixture was stirred at the same temperature for 2 h before the dropwise addition of Mel (1.7 mL, 27.750 mmol, 1.5 equiv). The resulting mixture was stirred at −78° C. for 30 min, then warmed to 0° C. in an ice bath and stirred for another 30 min. Then, EtOAc (150.0 mL) and 2 M HCl (50.0 mL, precooled at 0° C.) were added to the reaction mixture sequentially at 0° C. After 4 h at 0° C., the layers were separated, and the aqueous layer extracted with EtOAc (2×150.0 mL). The combined organic layers were washed with brine (150.0 mL), dried over anhydrous Na₂SO₄, filtered and concentrated in vacuo. dr value was determined to be 4:1 by ¹H NMR analysis of an aliquot. The crude residue was purified by flash chromatography (silica gel, eluent: 25% EtOAc/hexane) to afford 11 (2.887 g, 75% yield, 2:1 dr) as a yellow oil. To the above aqueous HCl layer was added EtOAc (150.0 mL), and the mixture was stirred at 0° C. for 2 h before the layers were separated. The aqueous layer was extracted with EtOAc (2×150.0 mL). The combined organic layers were washed with brine (150.0 mL), dried over anhydrous Na₂SO₄, filtered and concentrated in vacuo. The crude residue was purified by flash chromatography (silica gel, eluent: 25% EtOAc/hexane) to afford 11 (0.186 g, 5% yield, 1.5:1 dr) as a yellow oil. In total, 3.073 g of two inseparable diastereomers were obtained with a dr of 2:1 and a combined yield of 80%. Note: epimerization occurred (dr value decreased from 4:1 prior to purification to 2:1 after purification), likely due to elevated water bath temperature of the rotary evaporator (the temperature was accidentally raised to 40° C. while solvent was removed in vacuo).

The characterization data matched spectral values of 11 obtained above.

### (−)-Stemoamide and (−)-9a-epi-stemoamide ((−)-1 and (−)-9a-epi-1)

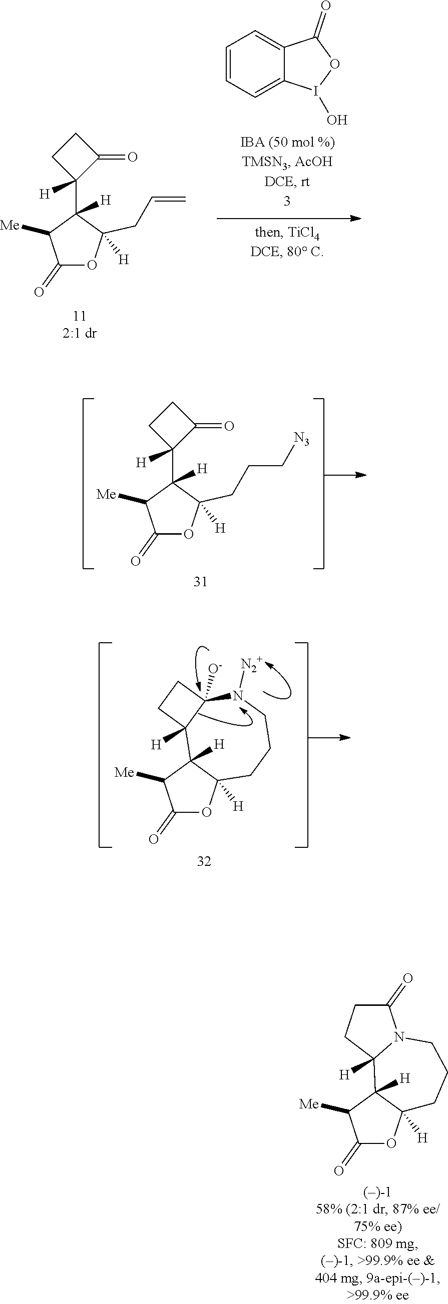

Following a modified literature procedure (H. Li, et al., Journal of the American Chemical Society 141, 9415-9421 (2019), X. Li, et al., Science China Chemistry 62, 1537-1541 (2019), and K. J. Frankowski, et al., Angewandte Chemie International Edition 54, 10555-10558 (2015)), to a 2:1 mixture of 11 (2.942 g, 14.130 mmol, 1 equiv) in anhydrous DCE (14.1 mL) were added iodosobenzoic acid (IBA) (1.903 g, 7.063 mmol, 50 mol %), azidotrimethylsilane (5.9 mL, 42.380 mmol, 3 equiv) and acetic acid (2.5 mL, 42.380 mmol, 3 equiv) sequentially under N₂ at room temperature. The mixture was stirred at room temperature for 20 h (100% conversion, reaction progress was monitored by ¹H NMR analysis of an aliquot). To the above reaction mixture was added anhydrous DCE (509.0 mL), followed by the dropwise addition TiCl₄ (1 M in DCE, 42.4 mL, 42.380 mmol, 3 equiv) at room temperature. The resulting mixture was heated at 80° C. for 24 h (100% conversion, reaction progress was monitored by ¹H NMR analysis of an aliquot) before being cooled to room temperature and quenched with saturated aqueous NaHCO₃ (300.0 mL). The mixture was filtered, and the layers were separated. The aqueous layer was extracted with EtOAc (3×150.0 mL). The combined organic layers were washed with brine (150.0 mL), dried over anhydrous Na₂SO₄, filtered and concentrated in vacuo. The crude residue was purified by flash chromatography (silica gel, eluent: 100% EtOAc to 5% MeOH in EtOAc) to afford a mixture of (−)-1 and (−)-9a-epi-1 (1.817 g, 58% yield, 2:1 dr. (−)-1: 87% ee; (−)-9a-epi-1: 75% ee) as a white solid. The mixture of stereoisomers was separated by preparative SFC purification performed at Lotus Separations, LLC (column=Chiralcel OJ-H 250×20 mm, temperature=35° C., CO₂/iPrOH=85/15, Flow rate=60 mL/min, back pressure=100 bar, UV=220 nm) to yield (−)-1 (809 mg, >99.9% ee) and (−)-9a-epi-1 (404 mg, >99.9% ee).

The characterization data matched spectral values of (−)-1 and (−)-9a-epi-1 obtained above.

### Synthesis of (−)-stemonine ((−)-3)

### Triisopropyl((3-methylfuran-2-yl)oxy)silane (33)

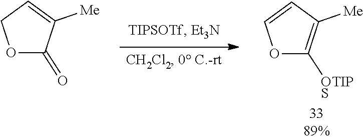

Following a known procedure (Y. Funakoshi, T. Miura, M. Murakami, S Organic Letters 18, 6284-6287 (2016)), to a solution of 3-methylfuran-2(5H)-one (90%, 484 μL, 5.000 mmol, 1 equiv) in anhydrous CH₂Cl₂ (5.0 mL) was added triethylamine (Et₃N) (1.4 mL, 10.000 mmol, 2 equiv) and triisopropylsilyltriflate (TIPSOTf) (1.6 mL, 6.000 mmol, 1.2 equiv) at 0° C. The mixture was stirred at room temperature for 15 h. Then it was quenched with water (20.0 mL) and extracted with EtOAc (2×20.0 mL). The organic layers were washed with brine (20.0 mL) and dried over anhydrous Na₂SO₄. After removal of solvent, the residue was purified by short-path vacuum distillation to yield 33 (1.130 g, 89% yield) as a colorless liquid.

¹H NMR (500 MHz, CDCl₃) δ 6.74 (d, J=2.2 Hz, 1H), 6.10 (d, J=2.2 Hz, 1H), 1.85 (s, 3H), 1.25 (sept, J=7.3 Hz, 3H), 1.09 (d, J=7.5 Hz, 18H). ¹³C NMR (125 MHz, CDCl₃) U 153.08, 130.64, 113.83, 91.51, 17.68, 12.47, 8.62.

The characterization data of 33 matched spectral values from literature.

### (1S,3aR,8S,10aS,10bR)-1-methyl-8-((S)-4-methyl-5-oxo-2,5-dihydrofuran-2-yl)decahydro-2H-furo[3,2-c]pyrrolo[1,2-a]azepin-2-one (34) and (1S,3aR,8S,10aS,10bR)-1-methyl-8-((R)-4-methyl-5-oxo-2,5-dihydrofuran-2-yl)decahydro-2H-furo[3,2-c]pyrrolo[1,2-a]azepin-2-one (13-epi-34)

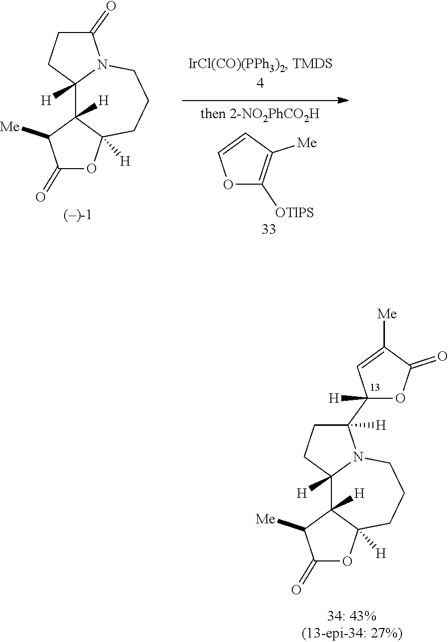

Following a literature procedure (M. Yoritate et al., Journal of the American Chemical Society 139, 18386-18391 (2017)), to a stirred solution of stemoamide (1) (22.3 mg, 0.10 mmol, 1 equiv) and Vaska's complex IrCl(CO)(PPh₃)₂ (0.8 mg, 0.001 mmol, 1 mol %) in anhydrous toluene (6.5 mL) was added 1,1,3,3-tetramethyldisiloxane (27 μL, 0.15 mmol, 1.5 equiv) at room temperature. The resulting solution was stirred for 1 h at the same temperature. Then, anhydrous MeCN (32.0 mL), triisopropyl((3-methylfuran-2-yl)oxy)silane 33 (71.2 mg, 0.28 mmol, 2.8 equiv) and 2-nitrobenzoic acid (83.6 mg, 0.5 mmol, 5 equiv) were added to the solution in sequence. After stirring for 1 day, the solution was acidified with aqueous 0.05 M HCl (10.0 mL). The mixture was extracted with aqueous 0.05 M HCl (3×10.0 mL). The combined aqueous layers were basified with aqueous saturated NaHCO₃ (10.0 mL) and extracted with CHCl₃ (3×30.0 mL). The combined organic extracts were dried over Na₂SO₄, and concentrated. The residue was purified by flash chromatography (silica gel, eluent: 50% EtOAc/hexanes to 100% EtOAc) to give 34 (more polar, 13.0 mg, 43%) and 13-epi-34 (less polar, 8.2 mg, 27%).

### Characterization Data of 34:

¹H NMR (500 MHz, CDCl₃) δ 6.99 (t, J=1.7 Hz, 1H), 4.81 (dt, J=6.1, 2.0 Hz, 1H), 4.19 (td, J 10.6, 3.6 Hz, 1H), 3.62 (dt, J 9.4, 5.9 Hz, 1H), 3.41-3.35 (m, 1H), 3.33 (dt, J=8.1, 6.4 Hz, 1H), 2.91 (ddd, J=16.0, 10.3, 2.3 Hz, 1H), 2.40 (dt, J=13.7, 6.9 Hz, 1H), 2.35-2.28 (m, 1H), 2.24 (ddd, J=12.4, 10.0, 5.3 Hz, 1H), 2.01-1.87 (comp, 4H), 1.86-1.78 (m, 1H), 1.60-1.49 (comp, 4H), 1.40 (qd, J 12.3, 5.2 Hz, 1H), 1.23 (d, J 6.9 Hz, 3H). ¹³C NMR (125 MHz, CDCl₃) δ 178.43, 174.14, 146.53, 131.40, 84.98, 79.06, 63.17, 58.29, 53.14, 46.52, 39.41, 34.47, 27.33, 26.73, 21.44, 14.05, 10.90. HRMS (ESI) calculated C₁₇H₂₄NO₄⁺ [M+H]⁺: 306.1700; found: 306.1697.

$${[ α ]}_{D}^{25.8} = - 29 8.43 ( c 1. , MeOH , 99 \% ee ) .$$

### Characterization Data of 13-Epi-34:

¹H NMR (500 MHz, CDCl₃) δ 7.04-6.97 (m, 1H), 4.98 (dt, J=3.9, 1.9 Hz, 1H), 4.25 (td, J=10.5, 3.5 Hz, 1H), 3.57 (dt, J=9.8, 5.8 Hz, 1H), 3.47 (td, J=6.8, 3.8 Hz, 1H), 3.10 (ddd, J=15.2, 5.4, 2.8 Hz, 1H), 2.89 (ddd, J=15.6, 11.1, 1.5 Hz, 1H), 2.43 (dq, J=12.2, 6.9 Hz, 1H), 2.36-2.28 (m, 1H), 2.20 (ddd, J=12.4, 10.0, 5.8 Hz, 1H), 1.94 (t, J=1.8 Hz, 3H), 1.84-1.74 (comp, 2H), 1.63-1.36 (comp, 5H), 1.23 (d, J=6.9 Hz, 3H). ¹³C NMR (125 MHz, CDCl₃) δ 178.58, 174.40, 146.79, 131.23, 82.13, 79.04, 62.77, 57.94, 52.99, 45.97, 39.66, 34.63, 27.04, 24.56, 23.32, 14.16, 10.99. HRMS (ESI) calculated C₁₇H₂₄NO₄⁺ [M+H]⁺: 306.1700; found: 306.1701.

$${[ α ]}_{D}^{25.4} = - 9 6.66 ( c 0.63 , MeOH , > 99 \% ee ) .$$

### (−)-Stemonine ((−)-3)

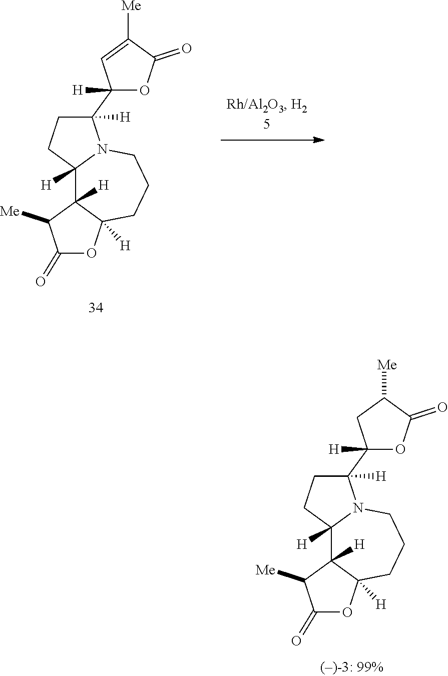

Following a literature procedure (M. Yoritate et al., Journal of the American Chemical Society 139, 18386-18391 (2017)), rhodium on alumina (5 wt %, 6.3 mg) was added to a solution of 34 (12.5 mg, 41 μmol, 1 equiv) in EtOH (1.0 mL). The flask was purged with hydrogen. The mixture was stirred under hydrogen atmosphere (1 atm) at room temperature for 3 h. Then it was filtered through a pad of Celite, washed with EtOAc, and concentrated. The residue was filtered through a pad of basic alumina, and then purified via flash chromatography (silica gel, eluent: 100% EtOAc to 99% EtOAc/MeOH) to afford stemonine (12.5 mg, 99%).

¹H NMR (700 MHz, CDCl₃) δ 4.22 (td, J=10.7, 3.2 Hz, 1H), 4.19-4.14 (m, 1H), 3.68 (dt, J=11.1, 5.7 Hz, 1H), 3.53 (dd, J=15.9, 4.9 Hz, 1H), 3.31 (q, J=7.6 Hz, 1H), 2.88 (dd, J=15.9, 11.6 Hz, 1H), 2.64-2.56 (m, 1H), 2.42 (dt, J=12.7, 6.8 Hz, 1H), 2.37 (ddd, J=13.1, 8.2, 5.6 Hz, 1H), 2.32 (dd, J=12.7, 3.4 Hz, 1H), 2.26 (ddd, J=11.9, 11.4, 5.4 Hz, 1H), 1.95 (dt, J=13.0, 6.7 Hz, 1H), 1.84 (dt, J=12.8, 6.7 Hz, 1H), 1.65 (ddd, J=16.1, 13.2, 7.4 Hz, 1H), 1.58-1.48 (comp, 3H), 1.46-1.37 (comp, 2H), 1.27 (d, J=7.0 Hz, 3H), 1.24 (d, J=6.9 Hz, 3H). ¹³C NMR (175 MHz, CDCl₃) δ 179.60, 178.54, 83.42, 79.11, 64.24, 58.67, 53.23, 46.43, 39.32, 35.01, 34.48, 34.47, 27.30, 26.70, 20.90, 15.04, 14.05. HRMS (ESI) calculated C₁₇H₂₆NO₄⁺ [M+H]⁺: 308.1856; found: 308.1855.

$${[ α ]}_{D}^{23.5} = - 94.43 ( c 0.2 , acetone , > 99 \% ee ) .$$

[literature values:

$${[ α ]}_{D}^{22} = - 1 8.6 ( c 0.2 , acetone , 98 \% ee ) ( 19 ) ;$$

$${[ α ]}_{D}^{21} = - 8 1.1 ( c 0.2 , acetone ) ( 35 ) ;$$

$${[ α ]}_{D}^{20} = - 1 2.5 ( c 0.08 , acetone ) ( 21 ) ]$$

### Example 3: Total Syntheses and Graph Edit Distance Analysis

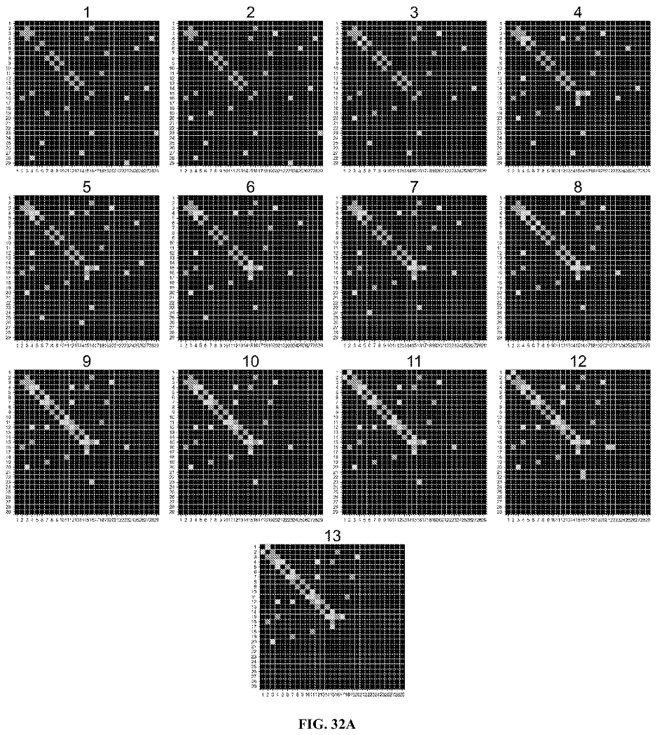

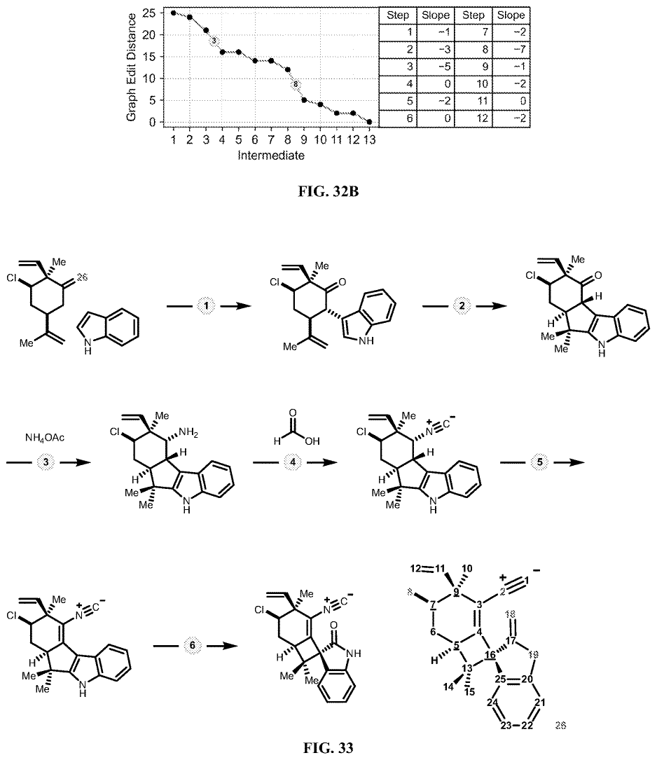

Total synthesis of (+)-welwitindolinone A by Baran and coworkers (P. S. Baran, T. J. Maimone, J. M. Richter, Nature 446, 404-408 (2007)) and atom mapping in the route are shown in FIG. 33, with the latter being used in constructing adjacency matrices and performing graph edit distance analysis in FIG. 34. Following General Procedures 1 and 2, the adjacency matrix for each starting material set, intermediate, and final product in total synthesis of (+)-welwitindolinone A by Baran and coworkers (FIG. 33) was computed and shown in FIG. 34, along with graph edit distance analysis of the synthetic route.

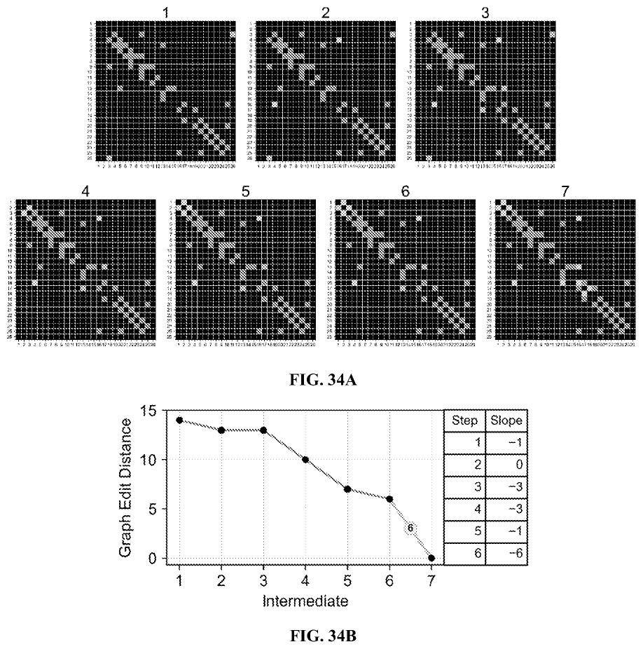

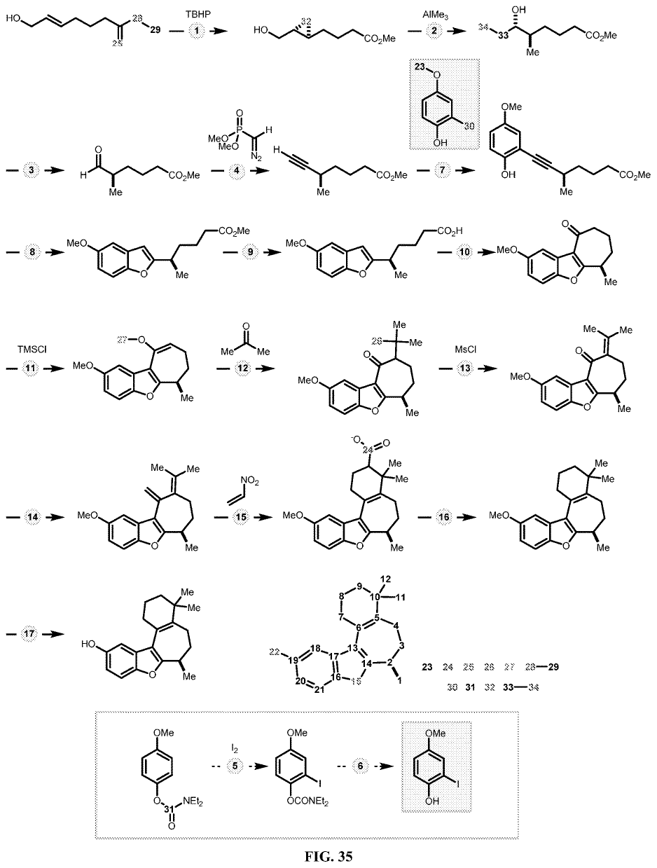

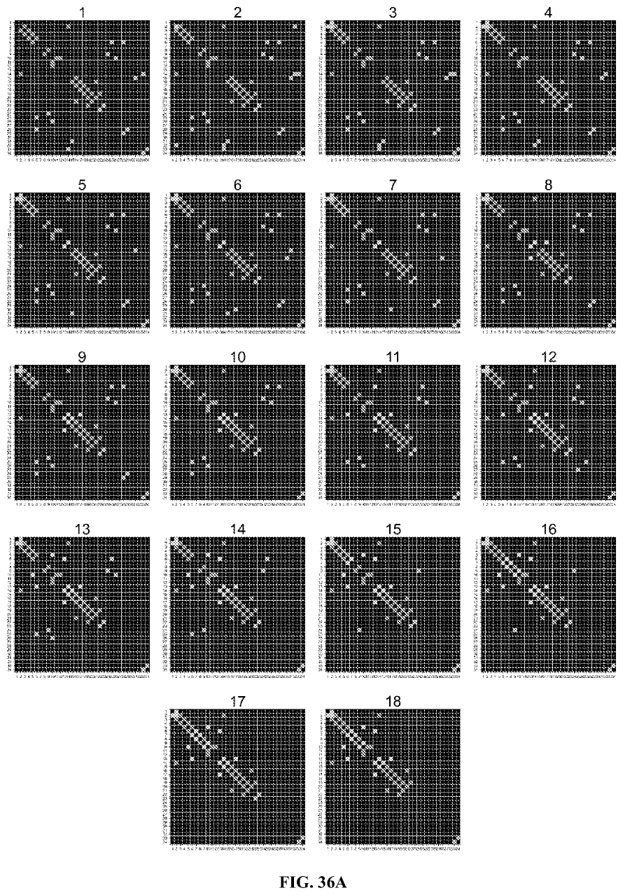

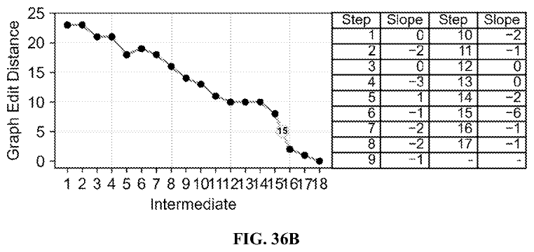

Total synthesis of (+)-frondosin B by Danishefsky and coworkers (M. Inoue, et al., Journal of the American Chemical Society 123, 1878-1889 (2001)) and atom mapping in the route are shown in FIG. 35, with the latter being used in constructing adjacency matrices and performing graph edit distance analysis in FIG. 36. Following General Procedures 1 and 2, the adjacency matrix for each starting material set, intermediate, and final product in total synthesis of (+)-frondosin B by Danishefsky and coworkers (FIG. 35) was computed and shown in FIG. 36, along with graph edit distance analysis of the synthetic route.

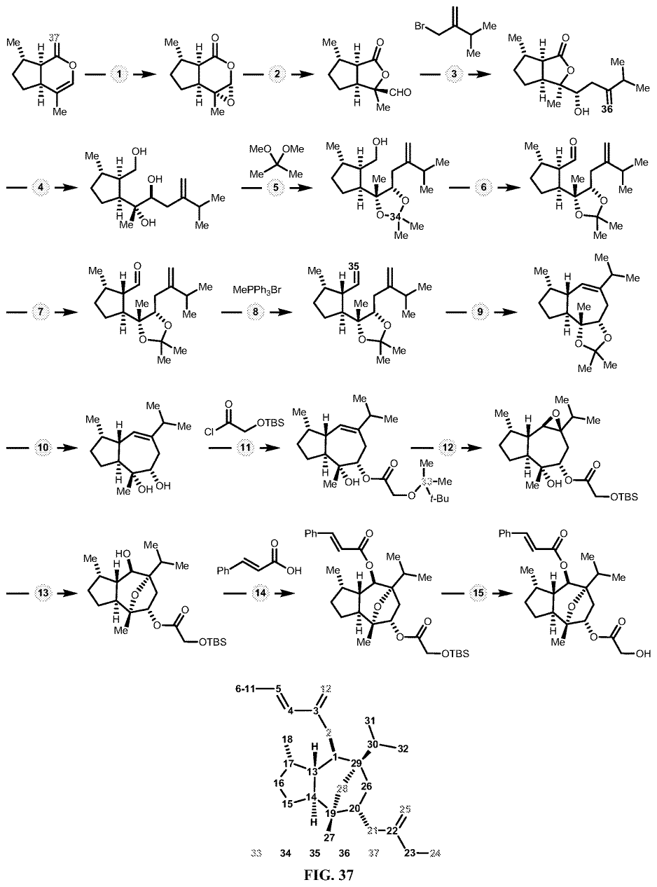

Total synthesis of (+)-englerin A by Christmann and coworkers (M. Willot et al., Angewandte Chemie International Edition 48, 9105-9108 (2009)) and atom mapping in the route are shown in FIG. 37, with the latter being used in constructing adjacency matrices and performing graph edit distance analysis in FIG. 38. Following General Procedures 1 and 2, the adjacency matrix for each starting material set, intermediate, and final product in total synthesis of (+)-englerin A by Christmann and coworkers (FIG. 37) was computed and shown in FIG. 38, along with graph edit distance analysis of the synthetic route.

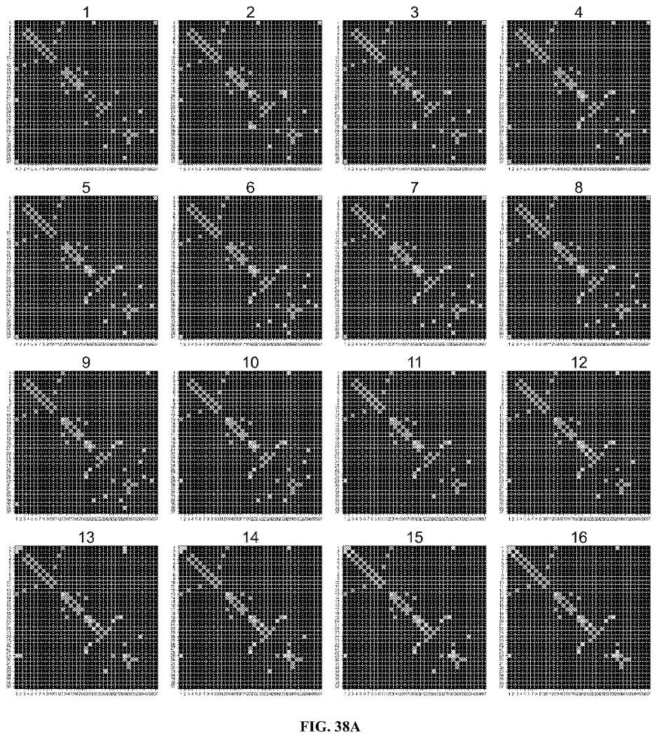

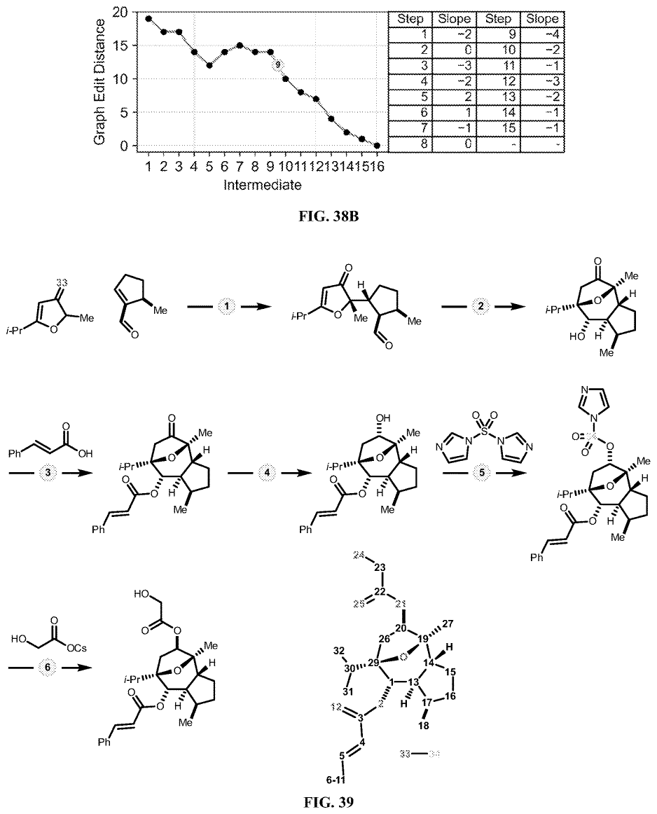

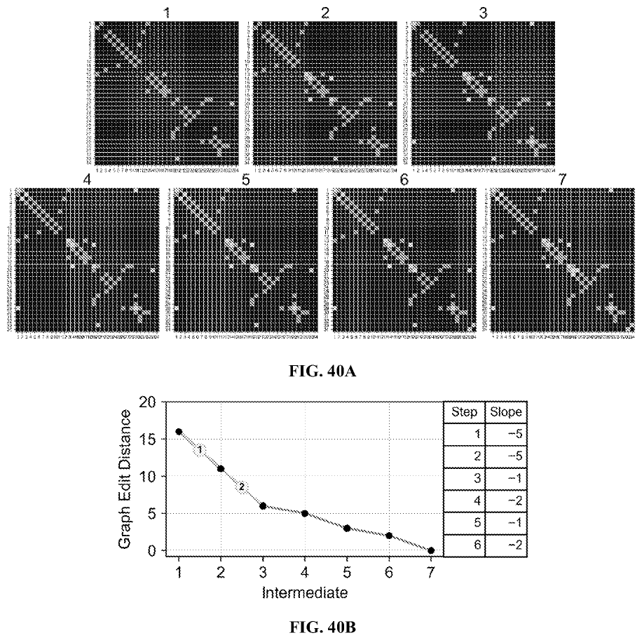

Total synthesis of (−)-englerin A by Chain and coworkers (M. Li, M. Nakashige, W. J. Chain, Journal of the American Chemical Society 133, 6553-6556 (2011)) and atom mapping in the route are shown in FIG. 39, with the latter being used in constructing adjacency matrices and performing graph edit distance analysis in FIG. 40. Following General Procedures 1 and 2, the adjacency matrix for each starting material set, intermediate, and final product in total synthesis of (−)-englerin A by Chain and coworkers (FIG. 39) was computed and shown in FIG. 40, along with graph edit distance analysis of the synthetic route.

Total synthesis of (−)-strychnine by MacMillan and coworkers (S. B. Jones, et al., Nature 475, 183-188 (2011)) and atom mapping in the route are shown in FIG. 41, with the latter being used in constructing adjacency matrices and performing graph edit distance analysis in FIG. 42. Following General Procedures 1 and 2, the adjacency matrix for each starting material set, intermediate, and final product in total synthesis of (−)-strychnine by MacMillan and coworkers (FIG. 41) was computed and shown in FIG. 42, along with graph edit distance analysis of the synthetic route.

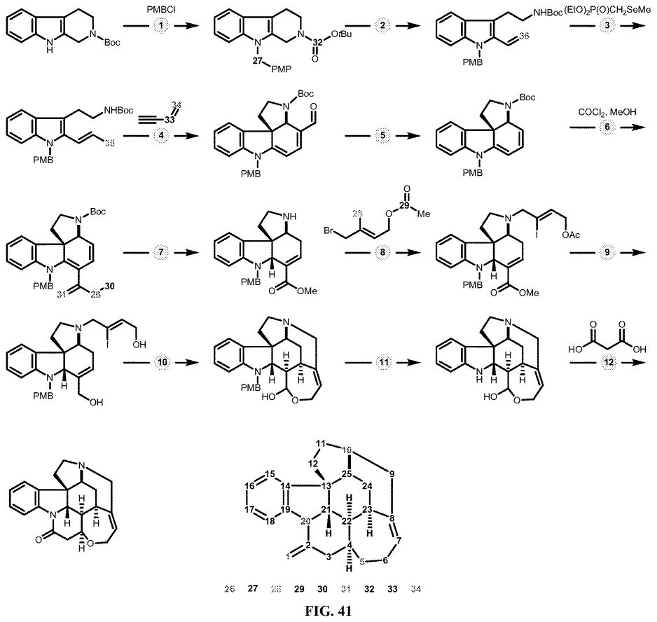

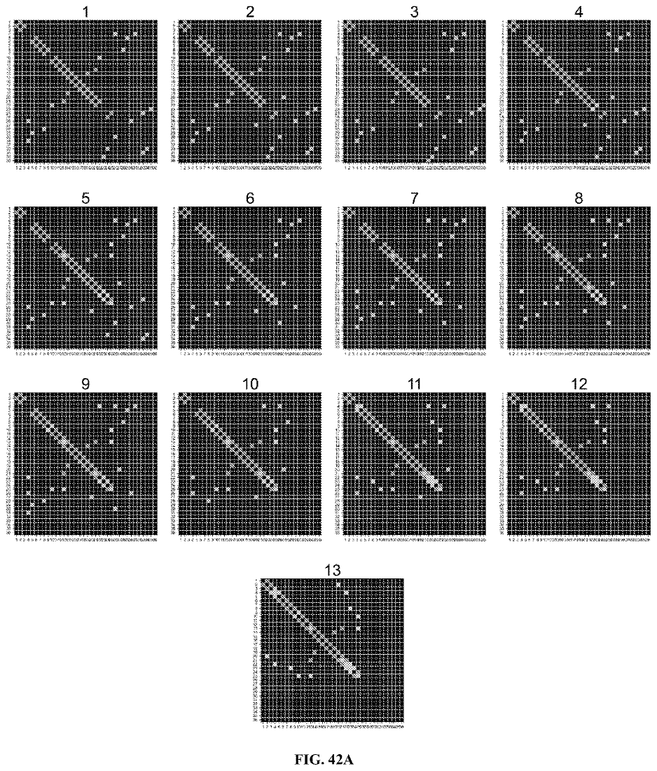

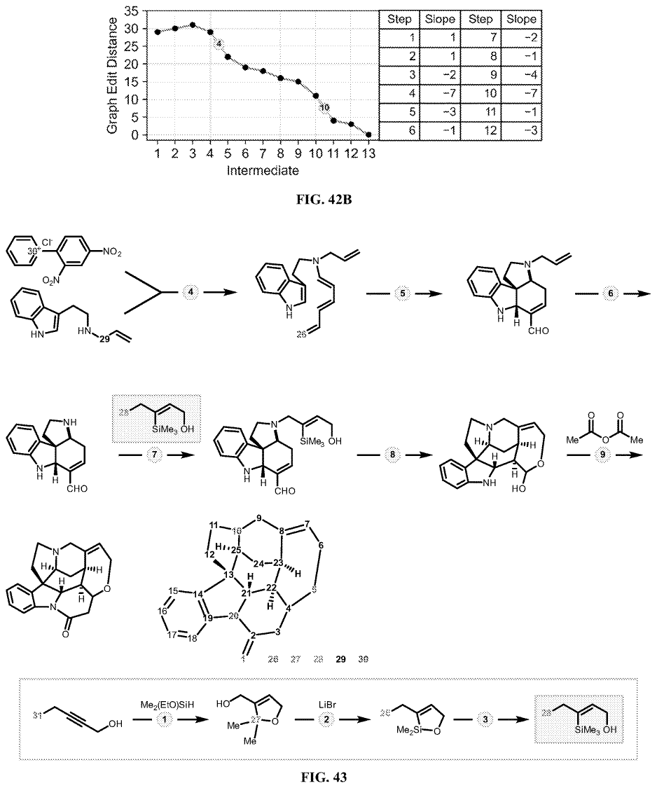

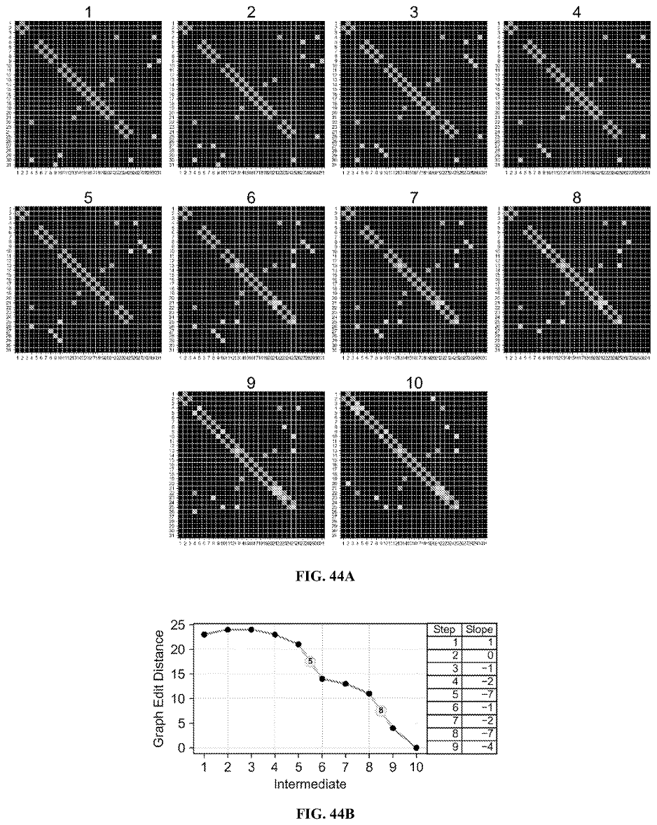

Total synthesis of (+)-strychnine by Vanderwal and coworkers (D. B. C. Martin, C. D. Vanderwal, Chemical Science 2, 649-651 (2011)) and atom mapping in the route are shown in FIG. 43, with the latter being used in constructing adjacency matrices and performing graph edit distance analysis in FIG. 44. Following General Procedures 1 and 2, the adjacency matrix for each starting material set, intermediate, and final product in total synthesis of (±)-strychnine by Vanderwal and coworkers (FIG. 43) was computed and shown in FIG. 44, along with graph edit distance analysis of the synthetic route.

Experimental Routes and their Graph Edit Distance Analysis

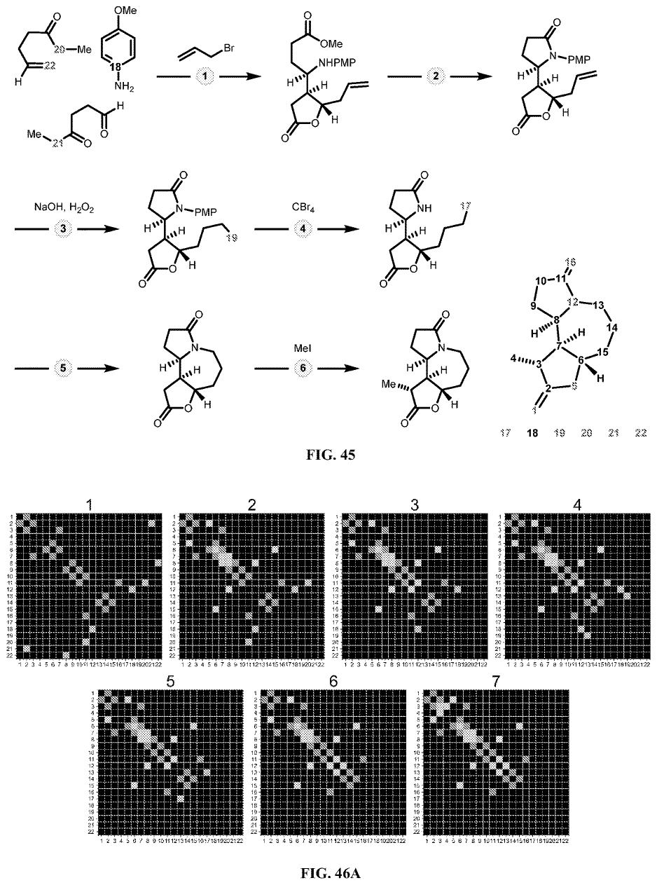

Total synthesis of (+)-stemoamide (FIG. 3) and atom mapping in the route are shown in FIG. 45, with the latter being used in constructing adjacency matrices and performing graph edit distance analysis in FIG. 46. Following General Procedures 1 and 2, the adjacency matrix for each starting material set, intermediate, and final product in total synthesis of (+)-stemoamide in this work (FIG. 45) was computed and shown in FIG. 46, along with graph edit distance analysis of the synthetic route.

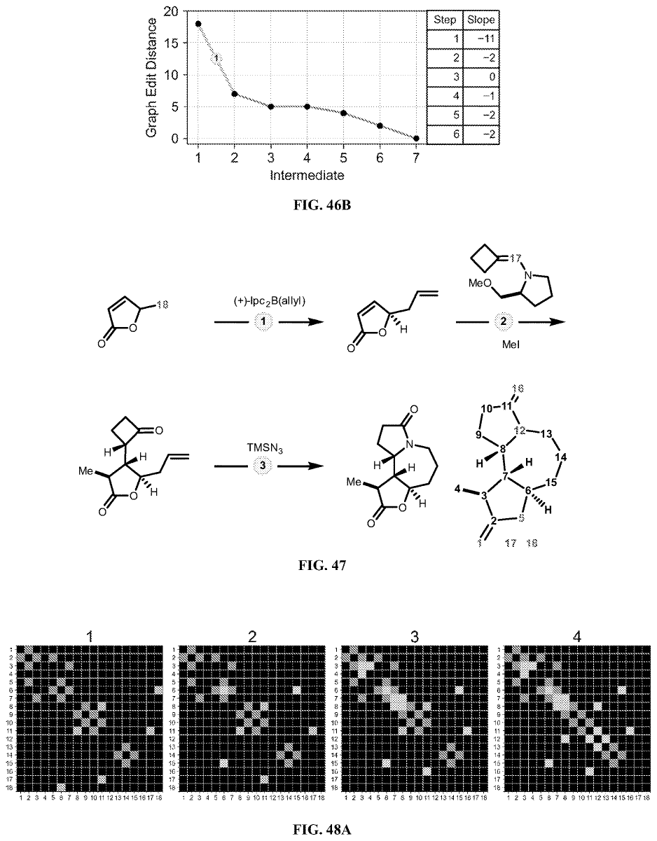

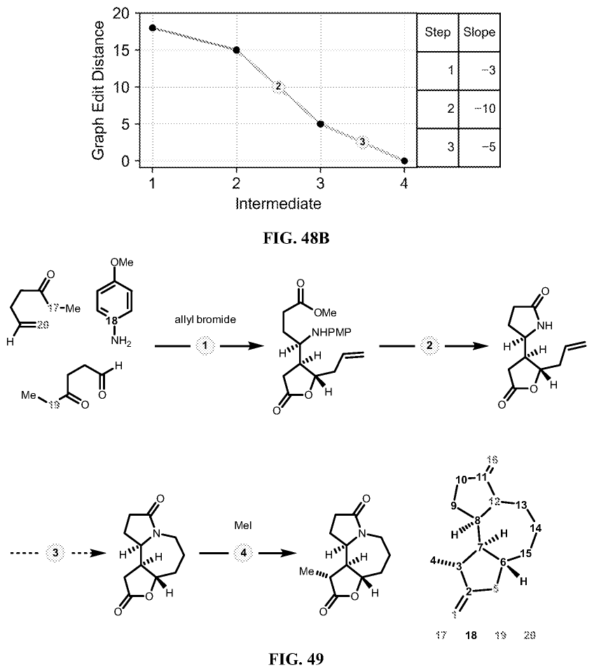

Total synthesis of (−)-stemoamide in this work and atom mapping in the route are shown in FIG. 47, with the latter being used in constructing adjacency matrices and performing graph edit distance analysis in FIG. 48. Following General Procedures 1 and 2, the adjacency matrix for each starting material set, intermediate, and final product in total synthesis of (−)-stemoamide in this work (FIG. 47) was computed and shown in FIG. 48, along with graph edit distance analysis of the synthetic route.

### Graph Edit Distance Analysis to Highlight Shortcuts

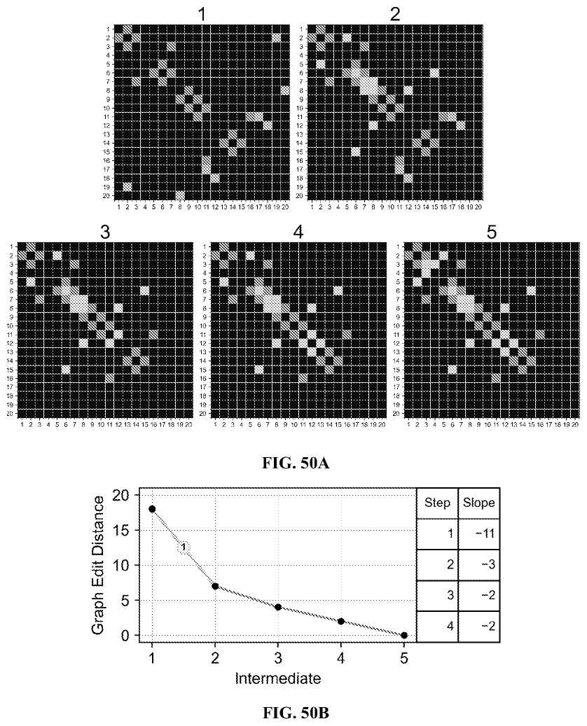

Graph edit distance analysis is used to highlight a shortcut in the experimental Mannich route to (+)-stemoamide, which identifies a new hypothetical anti-Markovnikov hydroamidation, ultimately leading to a 4-step synthesis of (+)-stemoamide. Total synthesis of (+)-stemoamide with the hypothetical anti-Markovnikov hydroamidation and atom mapping in the route are shown in FIG. 49, with the latter being used in constructing adjacency matrices and performing graph edit distance analysis in FIG. 50.

Following General Procedures 1 and 2, the adjacency matrix for each starting material set, intermediate, and final product in total synthesis of (+)-stemoamide with the hypothetical anti-Markovnikov hydroamidation (FIG. 49) was computed and shown in FIG. 50, along with graph edit distance analysis of the synthetic route.

1. A method for selecting a chemical synthesis process for a target molecule, comprising:
generating adjacency matrices for the target molecule and all synthetic intermediates for one or more routes of synthesis of the target molecule;
determining graph edit distances from the adjacency matrices; and
identifying a chemical synthesis process.

2. The method of claim 1, wherein the one or more routes of synthesis comprise a computer aided synthesis planning (CASP) route and/or an experimentally-determined route of synthesis.

3. (canceled)

4. The method of claim 1, wherein the methods comprises generating adjacency matrices and determining graph edit distances for two or more routes of synthesis for the target molecule.

5. The method of claim 4, wherein the two or more routes of synthesis are any combination of CASP and/or experimentally-determined routes of synthesis.

6. The method of claim 1, further comprising determining maximum common substructure (MCS) distance for the target molecule and all synthetic intermediates.

7. The method of claim 6, further comprising calculating a final distance metric based on the maximum common substructure (MCS) distance and the graph edit distances.

8. The method of claim 1, further comprising identifying one or more high impact steps in the one or more routes of synthesis.

9. The method of claim 8, wherein the high impact steps:
maximize the graph edit distance, the MCS distance, and/or the final distance metric between steps in the route of synthesis; and/or form two or more requisite target bonds simultaneously.

10. (canceled)

11. The method of claim 1, wherein the identified chemical synthesis process combines high impact steps from two or more different routes of synthesis for the target molecule.

12. The method of claim 1, further comprising identifying neighboring modest to low impact steps in the one or more routes of synthesis.

13. The method of claim 12, further comprising grouping the neighboring modest to low impact steps into fewer steps with higher impact.

14. The method of claim 13, wherein the neighboring modest to low impact steps are grouped into a single step.

15. The method of claim 12, wherein the identified chemical synthesis process minimizes neighboring modest to low impact steps.

16. The method of claim 1, wherein the identified chemical synthesis process has fewer steps than a previously developed synthesis process for the target molecule.

17. The method of claim 1, wherein the identified chemical synthesis decreases reagent costs, utilizes available starting or intermediate compounds or reagents, and/or increases yield when compared to a previously developed synthesis process for the target molecule.

18. The method of claim 1, further comprising synthesizing the target molecule according to the selected chemical synthesis process.

19. The method of claim 1, wherein the method is a computer-implemented method.

20. A non-transitory computer-readable medium storing instructions, that when executed by one or more processors performs operations comprising:
generating adjacency matrices for the target molecule and all synthetic intermediates for one or more routes of synthesis of the target molecule;
determining graph edit distances from the adjacency matrices; and
identifying a chemical synthesis process.

21. The non-transitory computer-readable medium of claim 20,
wherein the operations further comprise one or more of:
determining maximum common substructure (MCS) distance;
calculating a final distance metric based on the maximum common substructure (MCS) distance and the graph edit distances;
identifying one or more high impact steps in the one or more routes of synthesis;
identifying neighboring modest to low impact steps in the one or more routes of synthesis; and
grouping the neighboring modest to low impact steps into fewer steps with higher impact.

22. (canceled)

23. A system comprising:
one or more processors; and
the computer readable medium of claim 20.
